SEQUENCE LISTING (295 sequences)

SEQ ID NO: 1 - DNA, 214 bp, Homo sapiens
tgcggcccccctctgcccgctcctctgaccaacacctgctttgtctgcaggcaccacagtgctgttgcccctggtcattttctttggtctttgccttttatccctcctcttcattggtttaatgtatcgctaccaacggtggaagtccaagctctactccattggtgagtgggggctttgggagggagagggagctggtgggggtgagggagga

SEQ ID NO: 2 - DNA, 129 bp, Homo sapiens
gggctgagagaggaagtgaaatttatgargctttctttctttttcctcagtttgtgggaaatcgacacctgaaaaagaggtgagatgaaatgagagagttactcccaaatgtccctgaccattccttat

SEQ ID NO: 3 - DNA, 178 bp, Homo sapiens
acatttgagtttgttttctgtagctgtctgagcttctcttttctttctaggactgattgtgggtgtgacagccttgggtctactaataataggagtggtgaactgtgtcatcatgacccaggtgaaaagtaagagtccatccttccttccttcatccacttgttcaggaagcttttgt

SEQ ID NO: 4 - DNA, 135 bp, Homo sapiens
gatgtgcctgaggaagtcaatctcttacttgtcccctctcctctttatagagaagcccttgtgcctgcagagagaagccaaggtggtgagtgtctccactgccctctccccctcttcccctggtctccttcccgg

SEQ ID NO: 5 - RNA, 20 nt, Artificial Sequence
  Synthetic oligonucleotide
ccgcaguaccugcagaccag

SEQ ID NO: 6 - RNA, 20 nt, Artificial Sequence
  Synthetic oligonucleotide
guaccugcagaccagagagg

SEQ ID NO: 7 - RNA, 20 nt, Artificial Sequence
  Synthetic oligonucleotide
cugcagaccagagagguugc

SEQ ID NO: 8 - RNA, 20 nt, Artificial Sequence
  Synthetic oligonucleotide
acugauggaguagacuucgg

SEQ ID NO: 9 - RNA, 20 nt, Artificial Sequence
  Synthetic oligonucleotide
aguccuacuuacugauggag

SEQ ID NO: 10 - RNA, 20 nt, Artificial Sequence
  Synthetic oligonucleotide
ccaaaguccuacuuacugau

SEQ ID NO: 11 - RNA, 20 nt, Artificial Sequence
  Synthetic oligonucleotide
agauaaccaggggcaacagc

SEQ ID NO: 12 - RNA, 20 nt, Artificial Sequence
  Synthetic oligonucleotide
aggauagaaggcaaagaccu

SEQ ID NO: 13 - RNA, 20 nt, Artificial Sequence
  Synthetic oligonucleotide
ggcacauuaaacugaugaag

SEQ ID NO: 14 - RNA, 20 nt, Artificial Sequence
  Synthetic oligonucleotide
ggccuccaccggggauaucg

SEQ ID NO: 15 - RNA, 20 nt, Artificial Sequence
  Synthetic oligonucleotide
cuggagaacaaagaaacaag

SEQ ID NO: 16 - RNA, 20 nt, Artificial Sequence
  Synthetic oligonucleotide
aucccuacaaacuggagaac

SEQ ID NO: 17 - RNA, 20 nt, Artificial Sequence
  Synthetic oligonucleotide
ggcacgggaucccuacaaac

SEQ ID NO: 18 - RNA, 20 nt, Artificial Sequence
  Synthetic oligonucleotide
cuucucaccucuuugacagg

SEQ ID NO: 19 - RNA, 20 nt, Artificial Sequence
  Synthetic oligonucleotide
uggagucgucccuucucacc

SEQ ID NO: 20 - RNA, 20 nt, Artificial Sequence
  Synthetic oligonucleotide
cuccaacaaucagaccuagg

SEQ ID NO: 21 - RNA, 20 nt, Artificial Sequence
  Synthetic oligonucleotide
caaucagaccuaggaaaacg

SEQ ID NO: 22 - RNA, 20 nt, Artificial Sequence
  Synthetic oligonucleotide
agaccuaggaaaacggcagg

SEQ ID NO: 23 - RNA, 20 nt, Artificial Sequence
  Synthetic oligonucleotide
ccuuacuuuuccucugcacc

SEQ ID NO: 24 - RNA, 20 nt, Artificial Sequence
  Synthetic oligonucleotide
gagcagaaccuuacuuuucc

SEQ ID NO: 25 - RNA, 20 nt, Artificial Sequence
  Synthetic oligonucleotide
gacgagagcagaaccuuacu

SEQ ID NO: 26 - RNA, 20 nt, Artificial Sequence
  Synthetic oligonucleotide
ucagcagacccagugauguc

SEQ ID NO: 27 - RNA, 20 nt, Artificial Sequence
  Synthetic oligonucleotide
augaugcaguucaccagucc

SEQ ID NO: 28 - RNA, 20 nt, Artificial Sequence
  Synthetic oligonucleotide
ucaccaguccuaacaucagc

SEQ ID NO: 29 - RNA, 20 nt, Artificial Sequence
  Synthetic oligonucleotide
ccucugcaccaggaugaugc

SEQ ID NO: 30 - RNA, 20 nt, Artificial Sequence
  Synthetic oligonucleotide
uucucuacaaugaagagagg

SEQ ID NO: 31 - RNA, 20 nt, Artificial Sequence
  Synthetic oligonucleotide
ggcuucucuacaaugaagag

SEQ ID NO: 32 - RNA, 20 nt, Artificial Sequence
  Synthetic oligonucleotide
uguaggcaggagggcuucuc

SEQ ID NO: 33 - RNA, 20 nt, Artificial Sequence
  Synthetic oligonucleotide
acucaccaccuuggcaucuc

SEQ ID NO: 34 - RNA, 20 nt, Artificial Sequence
  Synthetic oligonucleotide
gcagagggauacucaccacc

SEQ ID NO: 35 - DNA, 16 bp, Artificial Sequence
  Synthetic oligonucleotide
caatcagacctaggaa

SEQ ID NO: 36 - DNA, 16 bp, Artificial Sequence
  Synthetic oligonucleotide
caacaatcagacctag

SEQ ID NO: 37 - DNA, 16 bp, Artificial Sequence
  Synthetic oligonucleotide
cagacctaggaaaacg

SEQ ID NO: 38 - DNA, 16 bp, Artificial Sequence
  Synthetic oligonucleotide
agcagacccagtgatg

SEQ ID NO: 39 - DNA, 16 bp, Artificial Sequence
  Synthetic oligonucleotide
ccagtcctaacatcag

SEQ ID NO: 40 - DNA, 16 bp, Artificial Sequence
  Synthetic oligonucleotide
caccagtcctaacatc

SEQ ID NO: 41 - DNA, 16 bp, Artificial Sequence
  Synthetic oligonucleotide
ctgcaccaggatgatg

SEQ ID NO: 42 - DNA, 16 bp, Artificial Sequence
  Synthetic oligonucleotide
acttttcctctgcacc

SEQ ID NO: 43 - DNA, 16 bp, Artificial Sequence
  Synthetic oligonucleotide
ccttacttttcctctg

SEQ ID NO: 44 - DNA, 16 bp, Artificial Sequence
  Synthetic oligonucleotide
cagaaccttacttttc

SEQ ID NO: 45 - DNA, 16 bp, Artificial Sequence
  Synthetic oligonucleotide
agagcagaaccttact

SEQ ID NO: 46 - DNA, 16 bp, Artificial Sequence
  Synthetic oligonucleotide
gagagcagaaccttac

SEQ ID NO: 47 - DNA, 16 bp, Artificial Sequence
  Synthetic oligonucleotide
accttacttttcctct

SEQ ID NO: 48 - DNA, 16 bp, Artificial Sequence
  Synthetic oligonucleotide
cttctctacaatgaag

SEQ ID NO: 49 - DNA, 16 bp, Artificial Sequence
  Synthetic oligonucleotide
ccttggcatctctttg

SEQ ID NO: 50 - DNA, 16 bp, Artificial Sequence
  Synthetic oligonucleotide
tcaccaccttggcatc

SEQ ID NO: 51 - DNA, 16 bp, Artificial Sequence
  Synthetic oligonucleotide
actcaccaccttggca

SEQ ID NO: 52 - DNA, 16 bp, Artificial Sequence
  Synthetic oligonucleotide
gatactcaccaccttg

SEQ ID NO: 53 - DNA, 16 bp, Artificial Sequence
  Synthetic oligonucleotide
ctacaatgaagagagg

SEQ ID NO: 54 - DNA, 16 bp, Artificial Sequence
  Synthetic oligonucleotide
ctctacaatgaagaga

SEQ ID NO: 55 - DNA, 16 bp, Artificial Sequence
  Synthetic oligonucleotide
agggatactcaccacc

SEQ ID NO: 56 - DNA, 16 bp, Artificial Sequence
  Synthetic oligonucleotide
cagagggatactcacc

SEQ ID NO: 57 - DNA, 16 bp, Artificial Sequence
  Synthetic oligonucleotide
cgcagagggatactca

SEQ ID NO: 58 - DNA, 16 bp, Artificial Sequence
  Synthetic oligonucleotide
gaacaagtcagaggca

SEQ ID NO: 59 - DNA, 16 bp, Artificial Sequence
  Synthetic oligonucleotide
gaggcaggacttcttc

SEQ ID NO: 60 - DNA, 16 bp, Artificial Sequence
  Synthetic oligonucleotide
cgcagtacctgcagac

SEQ ID NO: 61 - DNA, 16 bp, Artificial Sequence
  Synthetic oligonucleotide
agtacctgcagaccag

SEQ ID NO: 62 - DNA, 16 bp, Artificial Sequence
  Synthetic oligonucleotide
ggcaacagcaccgcag

SEQ ID NO: 63 - DNA, 16 bp, Artificial Sequence
  Synthetic oligonucleotide
ctagcaagataaccag

SEQ ID NO: 64 - DNA, 16 bp, Artificial Sequence
  Synthetic oligonucleotide
gcacattaaactgatg

SEQ ID NO: 65 - DNA, 16 bp, Artificial Sequence
  Synthetic oligonucleotide
cttcgggcctccaccg

SEQ ID NO: 66 - DNA, 16 bp, Artificial Sequence
  Synthetic oligonucleotide
cttactgatggagtag

SEQ ID NO: 67 - DNA, 16 bp, Artificial Sequence
  Synthetic oligonucleotide
cctacttactgatgga

SEQ ID NO: 68 - DNA, 16 bp, Artificial Sequence
  Synthetic oligonucleotide
gtcctacttactgatg

SEQ ID NO: 69 - DNA, 16 bp, Artificial Sequence
  Synthetic oligonucleotide
tccctacaaactggag

SEQ ID NO: 70 - DNA, 16 bp, Artificial Sequence
  Synthetic oligonucleotide
ggcacgggatccctac

SEQ ID NO: 71 - DNA, 16 bp, Artificial Sequence
  Synthetic oligonucleotide
ctctttgacaggcacg

SEQ ID NO: 72 - DNA, 16 bp, Artificial Sequence
  Synthetic oligonucleotide
ctcacctctttgacag

SEQ ID NO: 73 - DNA, 16 bp, Artificial Sequence
  Synthetic oligonucleotide
ccttctcacctctttg

SEQ ID NO: 74 - DNA, 16 bp, Artificial Sequence
  Synthetic oligonucleotide
ccacaatcagtcctag

SEQ ID NO: 75 - DNA, 16 bp, Artificial Sequence
  Synthetic oligonucleotide
cagtcctagaaagaaa

SEQ ID NO: 76 - DNA, 16 bp, Artificial Sequence
  Synthetic oligonucleotide
agtagacccaaggctg

SEQ ID NO: 77 - DNA, 16 bp, Artificial Sequence
  Synthetic oligonucleotide
ccactcctattattag

SEQ ID NO: 78 - DNA, 16 bp, Artificial Sequence
  Synthetic oligonucleotide
caccactcctattatt

SEQ ID NO: 79 - DNA, 16 bp, Artificial Sequence
  Synthetic oligonucleotide
ctgggtcatgatgaca

SEQ ID NO: 80 - DNA, 16 bp, Artificial Sequence
  Synthetic oligonucleotide
acttttcacctgggtc

SEQ ID NO: 81 - DNA, 16 bp, Artificial Sequence
  Synthetic oligonucleotide
tcttacttttcacctg

SEQ ID NO: 82 - DNA, 16 bp, Artificial Sequence
  Synthetic oligonucleotide
tggactcttacttttc

SEQ ID NO: 83 - DNA, 16 bp, Artificial Sequence
  Synthetic oligonucleotide
aggatggactcttact

SEQ ID NO: 84 - DNA, 16 bp, Artificial Sequence
  Synthetic oligonucleotide
aaggatggactcttac

SEQ ID NO: 85 - DNA, 16 bp, Artificial Sequence
  Synthetic oligonucleotide
cttctctataaagagg

SEQ ID NO: 86 - DNA, 16 bp, Artificial Sequence
  Synthetic oligonucleotide
ccttggcttctctctg

SEQ ID NO: 87 - DNA, 16 bp, Artificial Sequence
  Synthetic oligonucleotide
tcaccaccttggcttc

SEQ ID NO: 88 - DNA, 16 bp, Artificial Sequence
  Synthetic oligonucleotide
actcaccaccttggct

SEQ ID NO: 89 - DNA, 16 bp, Artificial Sequence
  Synthetic oligonucleotide
gacactcaccaccttg

SEQ ID NO: 90 - DNA, 16 bp, Artificial Sequence
  Synthetic oligonucleotide
tgtggtgcctgcagac

SEQ ID NO: 91 - DNA, 16 bp, Artificial Sequence
  Synthetic oligonucleotide
ggtgcctgcagacaaa

SEQ ID NO: 92 - DNA, 16 bp, Artificial Sequence
  Synthetic oligonucleotide
ggcaacagcactgtgg

SEQ ID NO: 93 - DNA, 16 bp, Artificial Sequence
  Synthetic oligonucleotide
caaagaaaatgaccag

SEQ ID NO: 94 - DNA, 16 bp, Artificial Sequence
  Synthetic oligonucleotide
atacattaaaccaatg

SEQ ID NO: 95 - DNA, 16 bp, Artificial Sequence
  Synthetic oligonucleotide
gcttggacttccaccg

SEQ ID NO: 96 - DNA, 16 bp, Artificial Sequence
  Synthetic oligonucleotide
ctcaccaatggagtag

SEQ ID NO: 97 - DNA, 16 bp, Artificial Sequence
  Synthetic oligonucleotide
cactcaccaatggagt

SEQ ID NO: 98 - DNA, 16 bp, Artificial Sequence
  Synthetic oligonucleotide
cccactcaccaatgga

SEQ ID NO: 99 - DNA, 16 bp, Artificial Sequence
  Synthetic oligonucleotide
cccccactcaccaatg

SEQ ID NO: 100 - DNA, 16 bp, Artificial Sequence
  Synthetic oligonucleotide
aaagcccccactcacc

SEQ ID NO: 101 - DNA, 16 bp, Artificial Sequence
  Synthetic oligonucleotide
tttcccacaaactgag

SEQ ID NO: 102 - DNA, 16 bp, Artificial Sequence
  Synthetic oligonucleotide
ggtgtcgatttcccac

SEQ ID NO: 103 - DNA, 16 bp, Artificial Sequence
  Synthetic oligonucleotide
ctctttttcaggtgtc

SEQ ID NO: 104 - DNA, 16 bp, Artificial Sequence
  Synthetic oligonucleotide
ctcacctctttttcag

SEQ ID NO: 105 - DNA, 16 bp, Artificial Sequence
  Synthetic oligonucleotide
tcatctcacctctttt

SEQ ID NO: 106 - DNA, 16 bp, Artificial Sequence
  Synthetic oligonucleotide
gctattaccttaaccc

SEQ ID NO: 107 - DNA, 214 bp, Mus musculus
cccctagtctctgctgtggcctcacactgagcaacctctctggtctgcaggtactgcggtgctgttgcccctggttatcttgctaggtctttgccttctatcctttatcttcatcagtttaatgtgccgatatccccggtggaggcccgaagtctactccatcagtaagtaggactttggggatatagggtgttggtggagatacgggaggggt

SEQ ID NO: 108 - DNA, 129 bp, Mus musculus
gcgttgaaagggaagtgaaattcatgacaccttgtttctttgttctccagtttgtagggatcccgtgcctgtcaaagaggtgagaagggacgactccagcttccctgactactccttccaacgcctgat

SEQ ID NO: 109 - DNA, 178 bp, Mus musculus
caccagccaccctggaacctttgtttctgagtaccctgccgttttcctaggtctgattgttggagtgacatcactgggtctgctgatgttaggactggtgaactgcatcatcctggtgcagaggaaaagtaaggttctgctctcgtcctgtttcccgccccacgtccctaccctaaca

SEQ ID NO: 110 - DNA, 135 bp, Mus musculus
ctgttctgaagaagtcctgcctctgacttgttcccctctcttcattgtagagaagccctcctgcctacaaagagatgccaaggtggtgagtatccctctgcggtcctcctcccccttctctcctccagctctccc

SEQ ID NO: 111 - DNA, 21 bp, Artificial Sequence
  Primer
gaaagtgagtgcgtcccttgc

SEQ ID NO: 112 - DNA, 20 bp, Artificial Sequence
  Primer
gcacggagcagagtgattcg

SEQ ID NO: 113 - DNA, 21 bp, Artificial Sequence
  Primer
gagccccaaatggaaatgtgc

SEQ ID NO: 114 - DNA, 17 bp, Artificial Sequence
  Primer
gctcaaggcctactgcc

SEQ ID NO: 115 - DNA, 24 bp, Artificial Sequence
  Primer
actgaaacatcagacgtggtgtgc

SEQ ID NO: 116 - DNA, 21 bp, Artificial Sequence
  Primer
ccttatcggcaggcaagtgag

SEQ ID NO: 117 - DNA, 1368 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 118 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 119 - DNA, 1386 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 120 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 121 - DNA, 1254 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcagtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatga

SEQ ID NO: 122 - protein, 417 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 123 - DNA, 1251 bp, Mus musculus
atgggtctccccaccgtgcctggcctgctgctgtcactggtgctcctggctctgctgatggggatacatccatcaggggtcactggactagtcccttctcttggtgaccgggagaagagggatagcttgtgtccccaaggaaagtatgtccattctaagaacaattccatctgctgcaccaagtgccacaaaggaacctacttggtgagtgactgtccgagcccagggcgggatacagtctgcagggagtgtgaaaagggcacctttacggcttcccagaattacctcaggcagtgtctcagttgcaagacatgtcggaaagaaatgtcccaggtggagatctctccttgccaagctgacaaggacacggtgtgtggctgtaaggagaaccagttccaacgctacctgagtgagacacacttccagtgcgtggactgcagcccctgcttcaacggcaccgtgacaatcccctgtaaggagactcagaacaccgtgtgtaactgccatgcagggttctttctgagagaaagtgagtgcgtcccttgcagccactgcaagaaaaatgaggagtgtatgaagttgtgcctacctcctccgcttgcaaatgtcacaaacccccaggactcagtttgtagggatcccgtgcctgtcaaagaggagaaggctggaaagcccctaactccagccccctccccagccttcagccccacctccggcttcaaccccactctgggcttcagcaccccaggctttagttctcctgtctccagtacccccatcagccccatcttcggtcctagtaactggcacttcatgccacctgtcagtgaggtagtcccaacccagggagctgaccctctgctctacgaatcactctgctccgtgccagcccccacctctgttcagaaatgggaagactccgcccacccgcaacgtcctgacaatgcagaccttgcgattctgtatgctgtggtggatggcgtgcctccagcgcgctggaaggagttcatgcgtttcatggggctgagcgagcacgagatcgagaggctggagatgcagaacgggcgctgcctgcgcgaggctcagtacagcatgctggaagcctggcggcgccgcacgccgcgccacgaggacacgctggaagtagtgggcctcgtgctttccaagatgaacctggctgggtgcctggagaatatcctcgaggctctgagaaatcccgccccctcgtccacgacccgcctcccgcgataa

SEQ ID NO: 124 - protein, 416 aa, Mus musculus
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSVCRDPVPVKEEKAGKPLTPAPSPAFSPTSGFNPTLGFSTPGFSSPVSSTPISPIFGPSNWHFMPPVSEVVPTQGADPLLYESLCSVPAPTSVQKWEDSAHPQRPDNADLAILYAVVDGVPPARWKEFMRFMGLSEHEIERLEMQNGRCLREAQYSMLEAWRRRTPRHEDTLEVVGLVLSKMNLAGCLENILEALRNPAPSSTTRLPR

SEQ ID NO: 125 - DNA, 1308 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttgagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 126 - protein, 435 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVEKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 127 - DNA, 1347 bp, Mus musculus
atggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattgagaagccctcctgcctacaaagagatgccaaggtgcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcctga

SEQ ID NO: 128 - protein, 448 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIEKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 129 - DNA, 178 bp, Homo sapiens; identical to SEQ ID NO: 3

SEQ ID NO: 130 - DNA, 16 bp, Artificial Sequence
  Synthetic  oligonucleotide
ccacaatcagtcctag

SEQ ID NO: 131 - DNA, 14 bp, Artificial Sequence
  Synthetic  oligonucleotide
acaatcagtcctag

SEQ ID NO: 132 - DNA, 12 bp, Artificial Sequence
  Synthetic oligonucleotide
aatcagtcctag

SEQ ID NO: 133 - DNA, 10 bp, Artificial Sequence
  Synthetic oligonucleotide
tcagtcctag

SEQ ID NO: 134 - DNA, 14 bp, Artificial Sequence
  Synthetic oligonucleotide
ccacaatcagtcct

SEQ ID NO: 135 - DNA, 12 bp, Artificial Sequence
  Synthetic oligonucleotide
ccacaatcagtc

SEQ ID NO: 136 - DNA, 10 bp, Artificial Sequence
  Synthetic oligonucleotide
ccacaatcag

SEQ ID NO: 137 - DNA, 14 bp, Artificial Sequence
  Synthetic oligonucleotide
cacaatcagtccta

SEQ ID NO: 138 - DNA, 12 bp, Artificial Sequence
  Synthetic  oligonucleotide
cacaatcagtcc

SEQ ID NO: 139 - DNA, 12 bp, Artificial Sequence
  Synthetic  oligonucleotide
acaatcagtcct

SEQ ID NO: 140 - DNA, 12 bp, Artificial Sequence
  Synthetic  oligonucleotide
caatcagtccta

SEQ ID NO: 141 - DNA, 10 bp, Artificial Sequence
  Synthetic oligonucleotide
cacaatcagt

SEQ ID NO: 142 - DNA, 10 bp, Artificial Sequence
  Synthetic oligonucleotide
acaatcagtc

SEQ ID NO: 143 - DNA, 10 bp, Artificial Sequence
  Synthetic oligonucleotide
caatcagtcc

SEQ ID NO: 144 - DNA, 10 bp, Artificial Sequence
  Synthetic oligonucleotide
aatcagtcct

SEQ ID NO: 145 - DNA, 10 bp, Artificial Sequence
  Synthetic oligonucleotide
atcagtccta

SEQ ID NO: 146 - DNA, 16 bp, Artificial Sequence
  Synthetic  oligonucleotide
cagtcctagaaagaaa

SEQ ID NO: 147 - DNA, 14 bp, Artificial Sequence
  Synthetic oligonucleotide
gtcctagaaagaaa

SEQ ID NO: 148 - DNA, 12 bp, Artificial Sequence
  Synthetic oligonucleotide
cctagaaagaaa

SEQ ID NO: 149 - DNA, 10 bp, Artificial Sequence
  Synthetic oligonucleotide
tagaaagaaa

SEQ ID NO: 150 - DNA, 14 bp, Artificial Sequence
  Synthetic  oligonucleotide
cagtcctagaaaga

SEQ ID NO: 151 - DNA, 12 bp, Artificial Sequence
  Synthetic oligonucleotide
cagtcctagaaa

SEQ ID NO: 152 - DNA, 10 bp, Artificial Sequence
  Synthetic oligonucleotide
cagtcctaga

SEQ ID NO: 153 - DNA, 14 bp, Artificial Sequence
  Synthetic oligonucleotide
agtcctagaaagaa

SEQ ID NO: 154 - DNA, 12 bp, Artificial Sequence
  Synthetic oligonucleotide
agtcctagaaag

SEQ ID NO: 155 - DNA, 12 bp, Artificial Sequence
  Synthetic oligonucleotide
gtcctagaaaga

SEQ ID NO: 156 - DNA, 12 bp, Artificial Sequence
  Synthetic oligonucleotide
tcctagaaagaa

SEQ ID NO: 157 - DNA, 10 bp, Artificial Sequence
  Synthetic oligonucleotide
agtcctagaa

SEQ ID NO: 158 - DNA, 10 bp, Artificial Sequence
  Synthetic oligonucleotide
gtcctagaaa

SEQ ID NO: 159 - DNA, 10 bp, Artificial Sequence
  Synthetic oligonucleotide
tcctagaaag

SEQ ID NO: 160 - DNA, 10 bp, Artificial Sequence
  Synthetic oligonucleotide
cctagaaaga

SEQ ID NO: 161 - DNA, 10 bp, Artificial Sequence
  Synthetic oligonucleotide
ctagaaagaa

SEQ ID NO: 162 - DNA, 16 bp, Artificial Sequence
  Synthetic oligonucleotide
acttttcacctgggtc

SEQ ID NO: 163 - DNA, 14 bp, Artificial Sequence
  Synthetic oligonucleotide
ttttcacctgggtc

SEQ ID NO: 164 - DNA, 12 bp, Artificial Sequence
  Synthetic oligonucleotide
ttcacctgggtc

SEQ ID NO: 165 - DNA, 10 bp, Artificial Sequence
  Synthetic oligonucleotide
cacctgggtc

SEQ ID NO: 166 - DNA, 14 bp, Artificial Sequence
  Synthetic oligonucleotide
acttttcacctggg

SEQ ID NO: 167 - DNA, 12 bp, Artificial Sequence
  Synthetic oligonucleotide
acttttcacctg

SEQ ID NO: 168 - DNA, 10 bp, Artificial Sequence
  Synthetic oligonucleotide
acttttcacc

SEQ ID NO: 169 - DNA, 14 bp, Artificial Sequence
  Synthetic oligonucleotide
cttttcacctgggt

SEQ ID NO: 170 - DNA, 12 bp, Artificial Sequence
  Synthetic oligonucleotide
cttttcacctgg

SEQ ID NO: 171 - DNA, 12 bp, Artificial Sequence
  Synthetic oligonucleotide
ttttcacctggg

SEQ ID NO: 172 - DNA, 12 bp, Artificial Sequence
  Synthetic oligonucleotide
tttcacctgggt

SEQ ID NO: 173 - DNA, 10 bp, Artificial Sequence
  Synthetic oligonucleotide
cttttcacct

SEQ ID NO: 174 - DNA, 10 bp, Artificial Sequence
  Synthetic oligonucleotide
ttttcacctg

SEQ ID NO: 175 - DNA, 10 bp, Artificial Sequence
  Synthetic oligonucleotide
tttcacctgg

SEQ ID NO: 176 - DNA, 10 bp, Artificial Sequence
  Synthetic oligonucleotide
ttcacctggg

SEQ ID NO: 177 - DNA, 10 bp, Artificial Sequence
  Synthetic oligonucleotide
tcacctgggt

SEQ ID NO: 178 - DNA, 16 bp, Artificial Sequence
  Synthetic oligonucleotide
agagcagaaccttact

SEQ ID NO: 179 - DNA, 10 bp, Artificial Sequence
  Synthetic oligonculeotide
gaacctuact

SEQ ID NO: 180 - DNA, 10 bp, Artificial Sequence
  Synthetic oligonucleotide
agagcagaac

SEQ ID NO: 181 - DNA, 10 bp, Artificial Sequence
  Synthetic oligonucleotide
gagcagaacc

SEQ ID NO: 182 - DNA, 10 bp, Artificial Sequence
  Synthetic oligonucleotide
agcagaacct

SEQ ID NO: 183 - DNA, 10 bp, Artificial Sequence
  Synthetic oligonculeotide
gcagaaccut

SEQ ID NO: 184 - DNA, 10 bp, Artificial Sequence
  Synthetic oligonculeotide
cagaacctua

SEQ ID NO: 185 - DNA, 10 bp, Artificial Sequence
  Synthetic oligonculeotide
agaaccutac

SEQ ID NO: 186 - DNA, 16 bp, Artificial Sequence
  Synthetic oligonucleotide
ccactcctattattag

SEQ ID NO: 187 - DNA, 16 bp, Artificial Sequence
  Synthetic oligonucleotide
caccactcctattatt

SEQ ID NO: 188 - DNA, 16 bp, Artificial Sequence
  Synthetic oligonucleotide
tggactcttacttttc

SEQ ID NO: 189 - DNA, 16 bp, Artificial Sequence
  Synthetic oligonucleotide
aaggatggactcttac

SEQ ID NO: 190 - DNA, 21 bp, Artificial Sequence
  Synthetic oligonucleotide
actgggcttcatcccagcatc

SEQ ID NO: 191 - DNA, 25 bp, Artificial Sequence
  Synthetic oligonucleotide
caccatggcgcccgtcgccgtctgg

SEQ ID NO: 192 - DNA, 40 bp, Artificial Sequence
  Synthetic oligonucleotide
cgacttcgctcttccagttgagaagcccttgtgcctgcag

SEQ ID NO: 193 - DNA, 24 bp, Artificial Sequence
  Synthetic oligonucleotide
ttaactgggcttcatcccagcatc

SEQ ID NO: 194 - DNA, 40 bp, Artificial Sequence
  Synthetic oligonucleotide
ctgcaggcacaagggcttctcaactggaagagcgaagtcg

SEQ ID NO: 195 - DNA, 21 bp, Artificial Sequence
  Synthetic oligonucleotide
ttaactgggcttcatcccagc

SEQ ID NO: 196 - DNA, 32 bp, Artificial Sequence
  Synthetic oligonucleotide
cgatagaattcatggcgcccgtcgccgtctgg

SEQ ID NO: 197 - DNA, 32 bp, Artificial Sequence
  Synthetic oligonucleotide
cctaactcgagttaactgggcttcatcccagc

SEQ ID NO: 198 - DNA, 39 bp, Artificial Sequence
  Synthetic oligonucleotide
gactgagcggccgccaccatggcgcccgtcgccgtctgg

SEQ ID NO: 199 - DNA, 38 bp, Artificial Sequence
  Synthetic oligonucleotide
ctaagcgcggccgcttaactgggcttcatcccagcatc

SEQ ID NO: 200 - DNA, 20 bp, Artificial Sequence
  Synthetic oligonucleotide
cgttctccaacacgacttca

SEQ ID NO: 201 - DNA, 21 bp, Artificial Sequence
  Synthetic oligonucleotide
cttatcggcaggcaagtgagg

SEQ ID NO: 202 - DNA, 24 bp, Artificial Sequence
  Synthetic oligonucleotide
actgaaacatcagacgtggtgtgc

SEQ ID NO: 203 - DNA, 21 bp, Artificial Sequence
  Synthetic oligonucleotide
ccttatcggcaggcaagtgag

SEQ ID NO: 204 - DNA, 22 bp, Artificial Sequence
  Synthetic oligonucleotide
cctcatctgagaagactgggcg

SEQ ID NO: 205 - DNA, 25 bp, Artificial Sequence
  Synthetic oligonucleotide
gccaccatgggcctctccaccgtgc

SEQ ID NO: 206 - DNA, 38 bp, Artificial Sequence
  Synthetic oligonucleotide
gggcactgaggactcagtttgtgggaaatcgacacctg

SEQ ID NO: 207 - DNA, 38 bp, Artificial Sequence
  Synthetic oligonucleotide
caggtgtcgatttcccacaaactgagtcctcagtgccc

SEQ ID NO: 208 - DNA, 23 bp, Artificial Sequence
  Synthetic oligonucleotide
caccatgggcctctccaccgtgc

SEQ ID NO: 209 - DNA, 17 bp, Artificial Sequence
  Synthetic oligonucleotide
tctgagaagactgggcg

SEQ ID NO: 210 - DNA, 30 bp, Artificial Sequence
  Synthetic oligonucleotide
cgataggatccatgggcctctccaccgtgc

SEQ ID NO: 211 - DNA, 31 bp, Artificial Sequence
  Synthetic oligonucleotide
cctaactcgagtcatctgagaagactgggcg

SEQ ID NO: 212 - DNA, 37 bp, Artificial Sequence
  Synthetic oligonucleotide
gactgagcggccgccaccatgggcctctccaccgtgc

SEQ ID NO: 213 - DNA, 34 bp, Artificial Sequence
  Synthetic oligonucleotide
ctaagcgcggccgctcatctgagaagactgggcg

SEQ ID NO: 214 - DNA, 20 bp, Artificial Sequence
  Synthetic oligonucleotide
ggtcaggccactttgactgc

SEQ ID NO: 215 - DNA, 19 bp, Artificial Sequence
  Synthetic oligonucleotide
caccgctgcccctatggcg

SEQ ID NO: 216 - DNA, 19 bp, Artificial Sequence
  Synthetic oligonucleotide
caccgctgccactatggcg

SEQ ID NO: 217 - DNA, 24 bp, Artificial Sequence
  Synthetic oligonucleotide
ggtcaggccactttgactgcaatc

SEQ ID NO: 218 - DNA, 27 bp, Artificial Sequence
  Synthetic oligonucleotide
gccaccatggcgcccgccgccctctgg

SEQ ID NO: 219 - DNA, 41 bp, Artificial Sequence
  Synthetic oligonucleotide
ggcatctctcttccaattgagaagccctcctgcctacaaag

SEQ ID NO: 220 - DNA, 41 bp, Artificial Sequence
  Synthetic oligonucleotide
ctttgtaggcaggagggcttctcaattggaagagagatgcc

SEQ ID NO: 221 - DNA, 21 bp, Artificial Sequence
  Synthetic oligonucleotide
ggccactttgactgcaatctg

SEQ ID NO: 222 - DNA, 25 bp, Artificial Sequence
  Synthetic oligonucleotide
caccatggcgcccgccgccctctgg

SEQ ID NO: 223 - DNA, 22 bp, Artificial Sequence
  Synthetic oligonucleotide
tcaggccactttgactgcaatc

SEQ ID NO: 224 - DNA, 32 bp, Artificial Sequence
  Synthetic oligonucleotide
cgatagaattcatggcgcccgccgccctctgg

SEQ ID NO: 225 - DNA, 33 bp, Artificial Sequence
  Synthetic oligonucleotide
cctaactcgagtcaggccactttgactgcaatc

SEQ ID NO: 226 - DNA, 39 bp, Artificial Sequence
  Synthetic oligonucleotide
gactgagcggccgccaccatggcgcccgccgccctctgg

SEQ ID NO: 227 - DNA, 36 bp, Artificial Sequence
  Synthetic oligonucleotide
ctaagcgcggccgctcaggccactttgactgcaatc

SEQ ID NO: 228 - DNA, 21 bp, Artificial Sequence
  Synthetic oligonucleotide
gagccccaaatggaaatgtgc

SEQ ID NO: 229 - DNA, 20 bp, Artificial Sequence
  Synthetic oligonucleotide
gctcaaggcctactgcatcc

SEQ ID NO: 230 - DNA, 22 bp, Artificial Sequence
  Synthetic oligonucleotide
ggttatcgcgggaggcgggtcg

SEQ ID NO: 231 - DNA, 26 bp, Artificial Sequence
  Synthetic oligonucleotide
gccaccatgggtctccccaccgtgcc

SEQ ID NO: 232 - DNA, 40 bp, Artificial Sequence
  Synthetic oligonucleotide
cacaaacccccaggactcagtttgtagggatcccgtgcct

SEQ ID NO: 233 - DNA, 40 bp, Artificial Sequence
  Synthetic oligonucleotide
aggcacgggatccctacaaactgagtcctgggggtttgtg

SEQ ID NO: 234 - DNA, 24 bp, Artificial Sequence
  Synthetic oligonucleotide
caccatgggtctccccaccgtgcc

SEQ ID NO: 235 - DNA, 20 bp, Artificial Sequence
  Synthetic oligonucleotide
tcgcgggaggcgggtcgtgg

SEQ ID NO: 236 - DNA, 31 bp, Artificial Sequence
  Synthetic oligonucleotide
cgatagtcgacatgggtctccccaccgtgcc

SEQ ID NO: 237 - DNA, 31 bp, Artificial Sequence
  Synthetic oligonucleotide
cctaagaattcttatcgcgggaggcgggtcg

SEQ ID NO: 238 - DNA, 38 bp, Artificial Sequence
  Synthetic oligonucleotide
gactgagcggccgccaccatgggtctccccaccgtgcc

SEQ ID NO: 239 - DNA, 34 bp, Artificial Sequence
  Synthetic oligonucleotide
ctaagcgcggccgcttatcgcgggaggcgggtcg

SEQ ID NO: 240 - DNA, 1347 bp, Mus musculus
atggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtagcactgaccaggtggagacccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccacagatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattgagaagccctcctgcctacaaagagatgccaaggtgcctcatgtgcctgatgagaaatcccaggatgcagtaggccttgagcagcagcacctgttgactacagcacccagttccagcagcagctccctagagagctcagccagcgctggggatcgaagggcgccccctgggggccatccccaagcaagagtcatggcggaggcccaagggtctcaggaggcccgcgccagctccaggatttcagattcttcccacggaagccacgggacccacgtcaacgtcacctgcatcgtgaacgtctgtagcagctctgaccacagctctcagtgctcttcccaagccagcgccacggtgggagacccagatgccaagccctcagcgtccccaaaggatgagcaggtccccttctctcaggaggagtgtccgtctcagtccccgtatgagactacagagacactgcagagccatgagaagcccttgccccttggtgtgccagatatgggcatgaagcccagccaagctggctggtttgatcagattgcagtcaaagtggcctga

SEQ ID NO: 241 - DNA, 1308 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttgagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccgggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa

SEQ ID NO: 242 - DNA, 144 bp, Artificial Sequence
  Synthetic oligonucleotide
aagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattga

SEQ ID NO: 243 - DNA, 16 bp, Artificial Sequence
  Synthetic oligonucleotide
caatcagacctaggaa

SEQ ID NO: 244 - DNA, 16 bp, Artificial Sequence
  Synthetic oligonucleotide
ccacaatcagtcctag

SEQ ID NO: 245 - DNA, 14 bp, Artificial Sequence
  Synthetic oligonucleotide
cacaatcagtccta

SEQ ID NO: 246 - DNA, 12 bp, Artificial Sequence
  Synthetic oligonucleotide
acaatcagtcct

SEQ ID NO: 247 - DNA, 214 bp, Homo sapiens
tcctccctcacccccaccagctccctctccctcccaaagcccccactcaccaatggagtagagcttggacttccaccgttggtagcgatacattaaaccaatgaagaggagggataaaaggcaaagaccaaagaaaatgaccaggggcaacagcactgtggtgcctgcagacaaagcaggtgttggtcagaggagcgggcagaggggggccgca

SEQ ID NO: 248 - DNA, 129 bp, Homo sapiens
ataaggaatggtcagggacatttgggagtaactctctcatttcatctcacctctttttcaggtgtcgatttcccacaaactgaggaaaaagaaagaaagcatcataaatttcacttcctctctcagccc

SEQ ID NO: 249 - DNA, 178 bp, Homo sapiens
acaaaagcttcctgaacaagtggatgaaggaaggaaggatggactcttacttttcacctgggtcatgatgacacagttcaccactcctattattagtagacccaaggctgtcacacccacaatcagtcctagaaagaaaagagaagctcagacagctacagaaaacaaactcaaatgt

SEQ ID NO: 250 - DNA, 135 bp, Homo sapiens
ccgggaaggagaccaggggaagagggggagagggcagtggagacactcaccaccttggcttctctctgcaggcacaagggcttctctataaagaggagaggggacaagtaagagattgacttcctcaggcacatc

SEQ ID NO: 251 - DNA, 14 bp, Artificial Sequence
  Synthetic oligonucleotide
acaatcagtcctag

SEQ ID NO: 252 - DNA, 12 bp, Artificial Sequence
  Synthetic oligonucleotide
aatcagtcctag

SEQ ID NO: 253 - DNA, 10 bp, Artificial Sequence
  Synthetic oligonucleotide
tcagtcctag

SEQ ID NO: 254 - DNA, 14 bp, Artificial Sequence
  Synthetic oligonucleotide
ccacaatcagtcct

SEQ ID NO: 255 - DNA, 12 bp, Artificial Sequence
  Synthetic oligonucleotide
ccacaatcagtc

SEQ ID NO: 256 - DNA, 10 bp, Artificial Sequence
  Synthetic oligonucleotide
ccacaatcag

SEQ ID NO: 257 - DNA, 12 bp, Artificial Sequence
  Synthetic oligonucleotide
cacaatcagtcc

SEQ ID NO: 258 - DNA, 12 bp, Artificial Sequence
  Synthetic oligonucleotide
caatcagtccta

SEQ ID NO: 259 - DNA, 10 bp, Artificial Sequence
  Synthetic oligonucleotide
cacaatcagt

SEQ ID NO: 260 - DNA, 10 bp, Artificial Sequence
  Synthetic oligonucleotide
acaatcagtc

SEQ ID NO: 261 - DNA, 10 bp, Artificial Sequence
  Synthetic oligonucleotide
caatcagtcc

SEQ ID NO: 262 - DNA, 10 bp, Artificial Sequence
  Synthetic oligonucleotide
aatcagtcct

SEQ ID NO: 263 - DNA, 10 bp, Artificial Sequence
  Synthetic oligonucleotide
atcagtccta

SEQ ID NO: 264 - DNA, 16 bp, Artificial Sequence
  Synthetic oligonucleotide
ccacaatcagtcctag

SEQ ID NO: 265 - DNA, 14 bp, Artificial Sequence
  Synthetic oligonucleotide
gtcctagaaagaaa

SEQ ID NO: 266 - DNA, 12 bp, Artificial Sequence
  Synthetic oligonucleotide
cctagaaagaaa

SEQ ID NO: 267 - DNA, 10 bp, Artificial Sequence
  Synthetic oligonucleotide
tagaaagaaa

SEQ ID NO: 268 - DNA, 14 bp, Artificial Sequence
  Synthetic oligonucleotide
cagtcctagaaaga

SEQ ID NO: 269 - DNA, 12 bp, Artificial Sequence
  Synthetic oligonucleotide
cagtcctagaaa

SEQ ID NO: 270 - DNA, 10 bp, Artificial Sequence
  Synthetic oligonucleotide
cagtcctaga

SEQ ID NO: 271 - DNA, 14 bp, Artificial Sequence
  Synthetic oligonucleotide
agtcctagaaagaa

SEQ ID NO: 272 - DNA, 12 bp, Artificial Sequence
  Synthetic oligonucleotide
agtcctagaaag

SEQ ID NO: 273 - DNA, 12 bp, Artificial Sequence
  Synthetic oligonucleotide
gtcctagaaaga

SEQ ID NO: 274 - DNA, 12 bp, Artificial Sequence
  Synthetic oligonucleotide
tcctagaaagaa

SEQ ID NO: 275 - DNA, 10 bp, Artificial Sequence
  Synthetic oligonucleotide
agtcctagaa

SEQ ID NO: 276 - DNA, 10 bp, Artificial Sequence
  Synthetic oligonucleotide
gtcctagaaa

SEQ ID NO: 277 - DNA, 10 bp, Artificial Sequence
  Synthetic oligonucleotide
tcctagaaag

SEQ ID NO: 278 - DNA, 10 bp, Artificial Sequence
  Synthetic oligonucleotide
cctagaaaga

SEQ ID NO: 279 - DNA, 10 bp, Artificial Sequence
  Synthetic oligonucleotide
ctagaaagaa

SEQ ID NO: 280 - DNA, 16 bp, Artificial Sequence
  Synthetic oligonucleotide
ccacaatcagtcctag

SEQ ID NO: 281 - DNA, 14 bp, Artificial Sequence
  Synthetic oligonucleotide
ttttcacctgggtc

SEQ ID NO: 282 - DNA, 12 bp, Artificial Sequence
  Synthetic oligonucleotide
ttcacctgggtc

SEQ ID NO: 283 - DNA, 10 bp, Artificial Sequence
  Synthetic oligonucleotide
cacctgggtc

SEQ ID NO: 284 - DNA, 14 bp, Artificial Sequence
  Synthetic oligonucleotide
acttttcacctggg

SEQ ID NO: 285 - DNA, 12 bp, Artificial Sequence
  Synthetic oligonucleotide
acttttcacctg

SEQ ID NO: 286 - DNA, 10 bp, Artificial Sequence
  Synthetic oligonucleotide
acttttcacc

SEQ ID NO: 287 - DNA, 14 bp, Artificial Sequence
  Synthetic oligonucleotide
cttttcacctgggt

SEQ ID NO: 288 - DNA, 12 bp, Artificial Sequence
  Synthetic oligonucleotide
cttttcacctgg

SEQ ID NO: 289 - DNA, 12 bp, Artificial Sequence
  Synthetic oligonucleotide
ttttcacctggg

SEQ ID NO: 290 - DNA, 12 bp, Artificial Sequence
  Synthetic oligonucleotide
tttcacctgggt

SEQ ID NO: 291 - DNA, 10 bp, Artificial Sequence
  Synthetic oligonucleotide
cttttcacct

SEQ ID NO: 292 - DNA, 10 bp, Artificial Sequence
  Synthetic oligonucleotide
ttttcacctg

SEQ ID NO: 293 - DNA, 10 bp, Artificial Sequence
  Synthetic oligonucleotide
tttcacctgg

SEQ ID NO: 294 - DNA, 10 bp, Artificial Sequence
  Synthetic oligonucleotide
ttcacctggg

SEQ ID NO: 295 - DNA, 10 bp, Artificial Sequence
  Synthetic oligonucleotide
tcacctgggt

(19) United States
(12) Patent Application Publication
(10) Pub. No.: US 2009/0264353 A1
(43) Pub. Date: Oct. 22, 2009

(54) Splice Switching Oligomers for TNF Superfamily Receptors and their Use in Treatment of Disease

(75) Inventors: Henrik Orum, Vaerlose (DK); Peter L. Sazani, Chapel Hill, NC (US)

Correspondence Address: FISH & RICHARDSON PC, P.O. BOX 1022, MINNEAPOLIS, MN, 55440-1022, US

(73) Assignee: Santaris Pharma A/S

(21) Appl. No.: 11/875,277

(22) Filed: Oct. 19, 2007

(51) Int. Cl. A61K 38/17; A61K 31/70; A61K 31/7052; C07H 19/00; C07K 14/47; C07H 21/02; C12N 15/85; C12N 5/10; C12P 21/02; A61P 29/00

(52) U.S. Cl. 514/12; 514/44 R; 536/22.1; 435/69.1; 530/350; 536/23.1; 435/320.1; 435/325

(57) ABSTRACT

The present invention relates to compositions and methods for preparing splice variants of TNFalpha receptor (TNFR) in vivo or in vitro, and the resulting TNFR protein variants. Such variants may be prepared by controlling the splicing of pre-mRNA molecules and regulating protein expression with splice switching oligonucleotides or splice switching oligomers (SSOs). The preferred SSOs according to the invention target exon 7 or 8 of TNFR1 (TNFRSF1A) or TNFR2 (TNFRSF1A) pre-mRNA, typically resulting in the production of TNFR variants which comprise a deletion in part or the entire exon 7 or 8 respectfully. SSOs targeting exon 7 are found to result in a soluble form of the TNFR, which has therapeutic benefit for treatment of inflammatory diseases. The SSO's are characterized in that they are substantially incapable or incapable of recruiting RNaseH.

This application claims priority to U.S. Ser. No. 60/862,350, filed Oct. 20, 2006; PCT/US2006/043651, filed Nov. 10, 2006; and U.S. Ser. No. 11/595,485, filed Nov. 10, 2006, which are all hereby incorporated by reference herein in their entirety.

### FIELD OF THE INVENTION

The present invention relates to compositions and methods for preparing splice variants of TNFalpha receptor (TNFR) in vivo or in vitro, and the resulting TNFR protein variants. Such variants may be prepared by controlling the splicing of pre-mRNA molecules and regulating protein expression with splice switching oligonucleotides or splice switching oligomers (SSOs). The preferred SSOs according to the invention target exon 7 or 8 of TNFR1 (TNFRSF1A) or TNFR2 (TNFRSF1A) pre-mRNA, typically resulting in the production of TNFR variants which comprise a deletion in part or the entire exon 7 or 8 respectfully. SSOs targeting exon 7 are found to result in a soluble form of the TNFR, which has therapeutic benefit for treatment of inflammatory diseases. The SSO's are characterized in that they are substantially incapable or incapable of recruiting RNaseH.

### BACKGROUND OF THE INVENTION

WO2007/05889, hereby incorporated by reference, provides a description of the background art relating to pre-mRNA splicing, the role of TNF-alpha in inflammation and inflammatory disorders, and the mediation of TNF-alpha activity via TNF1 and TNF2.

TNF-alpha is a pro-inflammatory cytokine that exists as a membrane-bound homotrimer and is released into the circulation by the protease TNF-alpha converting enzyme (TACE). TNF-alpha is introduced into the circulation as a mediator of the inflammatory response to injury and infection. TNF-alpha activity is implicated in the progression of inflammatory diseases such as rheumatoid arthritis, Crohn's disease, ulcerative colitis, psoriasis and psoriatic arthritis (Palladino, M. A., et al., 2003, Nat. Rev. Drug Discov. 2:736-46). The acute exposure to high levels of TNF-alpha, as experienced during a massive infection, results in sepsis; its symptoms include shock, hypoxia, multiple organ failure, and death. Chronic low doses of TNF-alpha can cause cachexia, a disease characterized by weight loss, dehydration and fat loss, and is associated with malignancies.

TNF-alpha activity is mediated primarily through two receptors coded by two different genes, TNFR1 and TNFR2. TNFR1 is a membrane-bound protein with a molecular weight of approximately 55 kilodaltons (kDa), while TNFR2 is a membrane-bound protein with a molecular weight of 75 kDa. The soluble extracellular domains of both receptors are shed to some extent from the cell membrane by the action of metalloproteases. Moreover, the pre-mRNA of TNFR2 undergoes alternative splicing, creating either a full length, active membrane-bound receptor (mTNFR2), or a secreted decoy receptor (sTNFR2) that lacks exons 7 and 8 which encompasses the coding sequences for the transmembrane (Lainez et al, 2004, Int. Immunol, 16:169). The sTNFR2 binds TNF-alpha but does not elicit a physiological response, thus reducing TNF-alpha activity. Although an endogenous, secreted splice variant of TNFR1 has not yet been identified, the similar gene structures of the two receptors strongly suggest the potential to produce this TNFR1 isoform.

Because of the role played by excessive activity by TNF superfamily members, it is useful to control the alternative splicing of TNFR receptors so that the amount of the secreted form is increased and the amount of the integral membrane form is decreased. The present invention provides splice switching oligonucleotides or splice switching oligomers (SSOs) to achieve this goal. SSOs are similar to antisense oligonucleotides (ASONs). However, in contrast to ASON, SSOs are able to hybridize to a target RNA without causing degradation of the target by RNase H

SSOs have been used to modify the aberrant splicing found in certain thalassemias (U.S. Pat. No. 5,976,879 to Kole; Lacerra, G., et al., 2000, Proc. Natl. Acad. Sci. 97:9591). Studies with the IL-5 receptor alpha-chain (IL-5Ralpha) demonstrated that SSOs directed against the membrane-spanning exon increased synthesis of the secreted form and inhibited synthesis of the integral membrane form (U.S. Pat. No. 6,210,892 to Bennett; Karras, J. G., et al., 2000, Mol. Pharm, 58:380). WO00/58512 also discloses examples of redirecting the splicing of IL-5R to soluble forms (examples 25 and 30).

SSOs have been used to produce the major CD40 splice variant detected in Tone, in which deletion of exon 6, which is upstream of the transmembrane region, resulted in an altered reading frame of the protein. While the SSO resulted in the expected mRNA splice variant, the translation product of the variant mRNA appeared to be unstable because the secreted receptor could not be detected (Siwkowski, A. M., et al., 2004, Nucleic Acids Res. 32; 2695). Tone et al., PNAS, 2001, 98(4):1751-1756 predicts that the mouse splice variant lacking exon 6 would not be a stable, secreted form of CD40 (see page 1756, right hand column.

WO02/088393 discloses gapmer oligonucleotides having 2′MOE wings and a deoxy gap, which are targeted to mouse TNFR2— these oligonucleotides are designed to recruit RNAseH to degrade the TNFR2 mRNA (mRNA down-regulation). The SSO oligonucleotides of the present invention are designed not to recruit RNaseH, but to disrupt the processing of the TNFR pre-mRNA, resulting in stable, secreted, ligand-binding TNFR splice variants.

US2005/202531 teaches that antisense oligonucleotides may be used to alter the alternative splicing pattern of CD40, however, it does not teach or provide any guidance as to splice elements or regions of CD40 that should be targeted by SSOs or any guidance as to which sequences should be used.

### SUMMARY OF THE INVENTION

The present invention employs splice switching oligonucleotides or splice switching oligomers (SSOs) to control the alternative splicing of receptors from the TNFR superfamily so that the amount of a soluble, stable, secreted, ligand-binding form is increased and the amount of the integral membrane form is decreased.

The invention provides an oligomer of between 8 and 50 nucleobases in length, comprising (or consisting) of a contiguous nucleobase sequence which consists of between 8 and 50 nucleobases in length, wherein said contiguous nucleobase sequence is complementary, preferably perfectly complementary, to a corresponding region of contiguous nucleotides present in SEQ ID NO 1 or SEQ ID NO 2, SEQ ID NO 3, or SEQ ID NO 4 and wherein said contiguous nucleobase sequence does not comprise 5 or more contiguous DNA (2′-deoxyribosnucleoside) monomer units.

SEQ ID NO 1 or SEQ ID NO 2, SEQ ID NO 3, or SEQ ID NO 4 are identical to SEQ ID NO 1 or SEQ ID NO 2, SEQ ID NO 3, or SEQ ID NO 4 of PCT/US2006/043651.

SEQ ID NO 247 is the reverse complement of SEQ ID NO 1. SEQ ID NO 248 is the reverse complement of SEQ ID NO 2, SEQ ID NO 249 is the reverse complement of SEQ ID NO 3, SEQ ID NO 250 is the reverse complement of SEQ ID NO 4.

Therefore, it is preferred that the oligomer of the invention comprises or consists of a contiguous nucleobase sequence which is homologous (preferably 100% homologus) to a corresponding region (i.e. part of) of SEQ ID NO 247, SEQ ID NO 248, SEQ ID NO 249, or SEQ ID NO 250.

The invention provides an oligomer of between 8 and 50 nucleobases in length, comprising (or consisting) of a contiguous nucleobase sequence which consists of between 8 and 50 nucleobases in length, wherein said contiguous nucleobase sequence is present in a (corresponding) region of contiguous nucleotides present in SEQ ID NO 247 or SEQ ID NO 248, SEQ ID NO 249, or SEQ ID NO 250 and wherein said contiguous nucleobase sequence does not comprise 5 or more contiguous DNA (2′-deoxyribosnucleoside) monomer units.

The invention provides an oligomer of between 8 and 50 nucleobases in length, comprising (or consisting) of a contiguous nucleobase sequence which consists of between 8 and 50 nucleobases in length, wherein said contiguous nucleobase sequence is complementary, preferably perfectly complementary, to a corresponding region of contiguous nucleotides present in SEQ ID NO 1 or SEQ ID NO 2, SEQ ID NO 3, or SEQ ID NO 4 and wherein said oligomer is essentially incapable, or incapable, of recruiting RNAseH when formed in a duplex with a complex with a complementary mRNA molecule.

The invention provides an oligomer of between 8 and 50 nucleobases in length, comprising (or consisting) of a contiguous nucleobase sequence which consists of between 8 and 50 nucleobases in length, wherein said contiguous nucleobase sequence is present in a (corresponding) region of contiguous nucleotides present in SEQ ID NO 247 or SEQ ID NO 248, SEQ ID NO 249, or SEQ ID NO 250 and wherein said oligomer is essentially incapable, or incapable, of recruiting RNAseH when formed in a duplex with a complex with a complementary mRNA molecule.

The invention further provides for a conjugate comprising the oligomer according to the invention and at least one non-nucleotide moiety covalently attached to said oligomer.

The invention further provides for pharmaceutical composition comprising the oligomer or the conjugate according to the invention and a pharmaceutically acceptable carrier.

The invention further provides for a method of altering the splicing of a TNFalpha receptor pre-mRNA mRNA, selected from TNFRSF1A or TNFRSF1A in a mammalian cell which expresses TNFRSF1A TNFalpha receptor or TNFRSF1B TNFalpha receptor, said method comprising administering an oligomer or a conjugate, or a pharmaceutical composition according to the invention to the cell.

The invention also refers to a method of preparing a soluble form of TNFRSF1A TNFalpha receptor or TNFRSF1B TNFalpha receptor in a mammalian cell which expresses said TNFalpha receptor, said method comprising administering an oligomer or a conjugate, or a pharmaceutical composition according to the invention to the cell.

The above methods may further comprise the step of purifying the soluble form of the TNFRSF1A TNFalpha receptor or the TNFRSF1B TNFalpha receptor.

The invention provides for a method of increasing the expression of a soluble form of TNFRSF1A TNFalpha receptor or TNFRSF1B TNFalpha receptor in a mammalian cell which expresses said TNFalpha receptor, said method comprising administering an oligomer or a conjugate, or a pharmaceutical composition according to the invention to the cell.

The above methods may be performed in vivo or in vitro.

The invention provides for a use of an oligomer according to the invention for the preparation of a medicament for the treatment of an inflammatory disease or condition.

The invention provides for a conjugate according to the invention for the treatment of an inflammatory disease or condition.

The invention provides for a method of treatment or prevention of an inflammatory disease or condition comprising the steps of administering the pharmaceutical composition according to the invention to a patient who is suffering from, or is likely to suffer from said inflammatory disease.

The invention provides for an isolated or purified soluble form of TNFalpha receptor comprises a deletion in the trans-membrane binding domain encoded by exon 7, wherein said TNFalpha receptor is selected from the TNFalpha receptor TNFRSF1A or TNFRSF1B.

The invention provides for an isolated or purified soluble form of TNFalpha receptor which lacks the trans-membrane binding domain encoded by exon 7, wherein said TNFalpha receptor is selected from the TNFalpha receptor TNFRSF1A or TNFRSF1B.

The invention further provides for a nucleic acid encoding the soluble form of TNFalpha receptor.

The invention further provides for a vector comprising the nucleic acid according to the invention, such as an expression vector.

The invention further provides for a host cell which comprises the nucleic acid or the vector according to the invention.

The invention further provides for a method for the preparation of a soluble form of TNFalpha receptor, said method comprising the step of culturing the host cell according to the invention under conditions which allow the expression of the nucleic acid according to the invention, and subsequently isolating said soluble form of TNFalpha receptor from said host cells.

The invention further provides for pharmaceutical composition comprising the isolated or purified soluble form of TNFalpha receptor according to the invention, or as prepared according to a method of the invention, and a pharmaceutically acceptable carrier.

The invention further provides for the use of the isolated or purified soluble form of TNFalpha receptor according to the invention, or as prepared according to a method of the invention, for the preparation of a medicament for the treatment of an inflammatory disease or condition.

The invention further provides for an isolated or purified soluble form of TNFalpha receptor according to the invention, or as prepared according to a method of the invention, for the treatment of an inflammatory disease or condition.

Related cases PCT/US2006/043651, PCT/US2007/10557, US11/595,485, and U.S. Ser. No. 11/799,117, are all hereby incorporated by reference herein in their entirety.

### BRIEF DESCRIPTION OF THE DRAWINGS

The following figures are identical to those described in PCT/US2006/043651 (FIGS. 1-20) and PCT/US2007/10557 (FIGS. 21-39), although reference numbers have been rearranged accordingly. FIG. 40 is new to the present application.

FIG. 1 depicts the structure of a portion of the tumor necrosis factor receptor pre-mRNA and spliced products for TNFR1 and TNFR2. These transcripts normally contain exon 7 and exon 8, which code for the transmembrane domain of the receptors. SSOs (bars) directed towards either or both of these exons elicit alternative splicing events, resulting in transcripts that lack the full transmembrane domain.

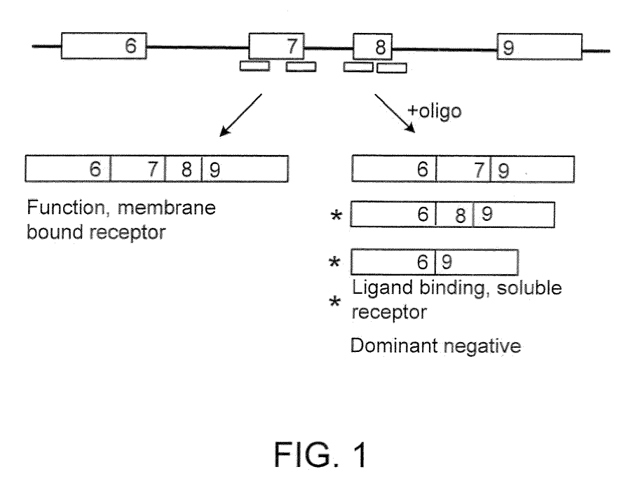

FIG. 2 shows the splicing products of SSOs for murine TNFR1 in cell culture. NIH-3T3 cells were mock transfected [Lipofectamine® 2000 (LFA2000 Only)] or transfected with the indicated concentration of either an exon 7 skipping TNFR1 SSO, A7-5 or A7-10, alone or a combination of exon 7 skipping SSO and an exon 8 skipping SSO, A8-3. Total RNA was isolated and RT-PCR performed 24 hours later. The PCR primers were used to amplify from Exon 5 to Exon 9, so that “Full Length” TNFR1 is represented by a 475 bp band. Transcripts lacking exon 7 (Delta Exon 7) and lacking both exon 7 and exon 8 (Delta Exon 7/8) are represented by 361 bp and 332 bp bands, respectively.

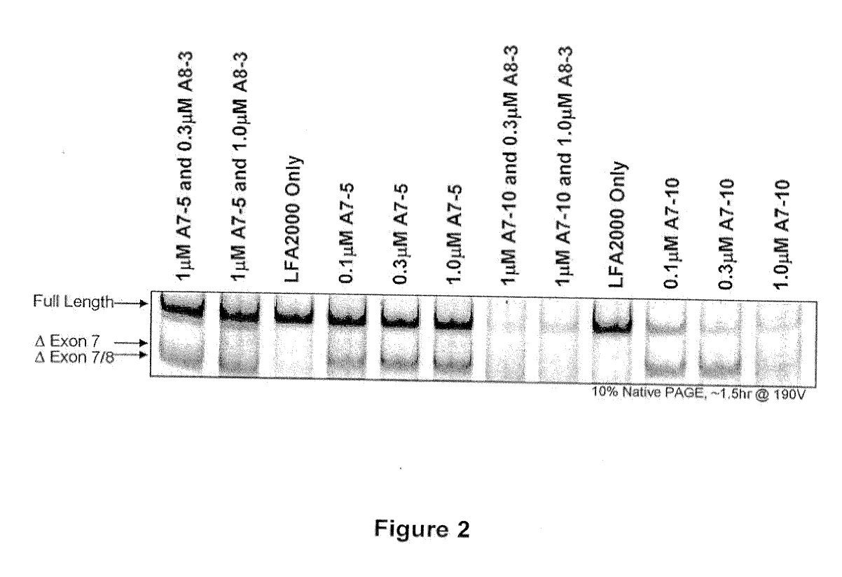

FIG. 3 shows the splicing products of SSOs for murine TNFR2 in cell culture. NIH-3T3 cells were mock transfected (LFA2000 Only) or transfected with the indicated concentration of either an exon 7 skipping TNFR2 SSO, B7-6 or B7-1, alone or a combination of exon 7 skipping oligonucleotide and an exon 8 skipping oligonucleotide, B 8-4. Total RNA was isolated and RT-PCR performed 24 hours later. The PCR primers were used to amplify from Exon 5 to Exon 9, so that “Full Length” TNFR2 is represented by a 486 bp band. Transcripts lacking exon 7 (Delta Exon 7) and lacking both exon 7 and exon 8 (Delta Exon 7/8) are represented by 408 bp and 373 bp bands, respectively.

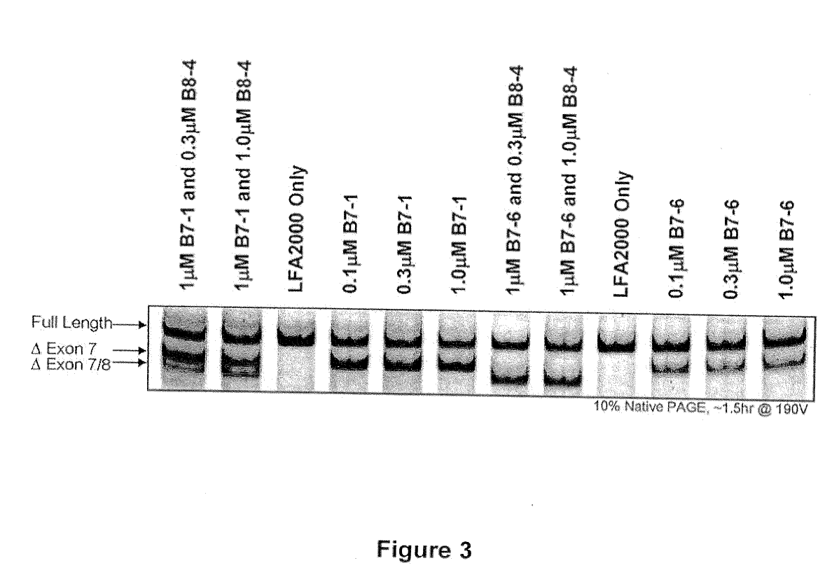

FIGS. 4A and 4B present the sequences of exons 7 (4A) and 8 (4B) of murine TNFR1 and of the flanking introns. Also shown are the sequences of 2O-Me-oligoribonucleoside-phosphorothioate SSOs that were assayed for splice switching activity.

FIGS. 5A and 5B present the sequences of exons 7 (5A) and 8 (5B) of murine TNFR2 and of the flanking introns. Also shown are the sequences of 2O-Me-oligoribonucleoside-phosphorothioate SSOs that were assayed for splice switching activity.

FIG. 6 provides an alignment of the human and murine TNF receptor genes in the regions that encode the transmembrane exons. The murine sequences, SEQ ID Nos: 107, 108, 109, and 110, are homologous to the human sequences, SEQ ID Nos: 1, 2, 3, and 4, respectively.

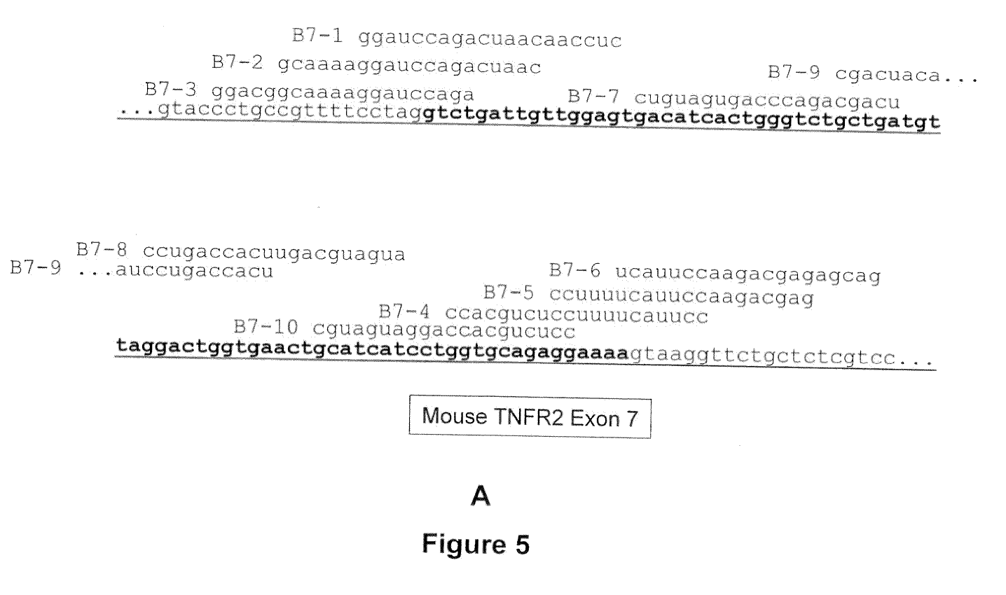

FIG. 7 shows the splicing products of SSOs for primary mouse hepatocyte cultures, in assays conducted as described in FIGS. 2 and 3.

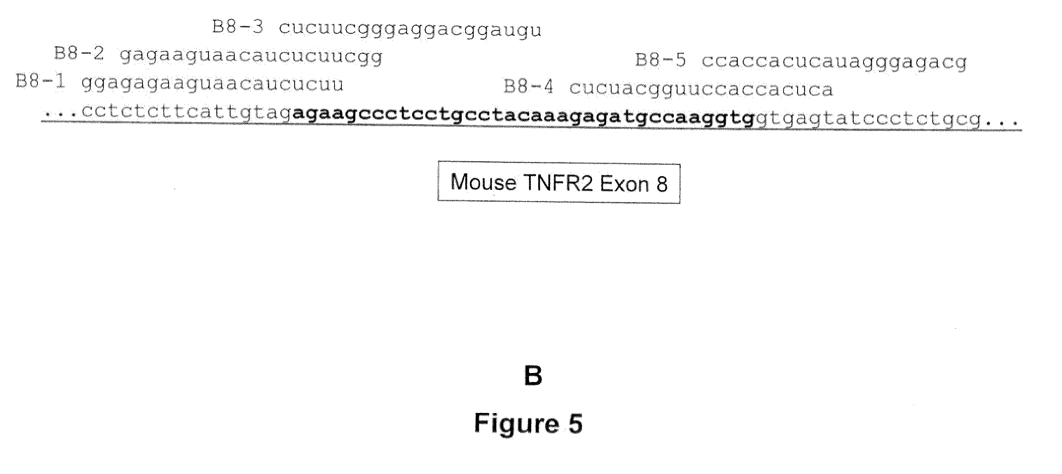

FIGS. 8A-8D provide mouse and human TNFR2 (TNFRSF1B) (8A and 8B) and TNFR1 (TNFRSF1A) (8C and 8D) LNA SSO sequences from Tables 2 and 3. FIGS. 8A and 8C schematically illustrate the position of each SSO relative to the targeted exon. FIGS. 8B and 8D show the pre-mRNA sequence (5′ to 3′) and the SSOs (3′ to 5′) hybridized to it.

FIG. 9 shows the splicing products for L929 murine cells treated with LNA SSOs. Cells were transfected with the indicated LNA SSO at a final concentration of 50 nM. After 24 hours, the cells were lysed and analyzed for splice switching by RT-PCR. Top panel, SSOs targeted to exon 7; bottom panel, SSOs targeted to exon 8. FL₅ full length TNFR2 amplicon; Delta7, Delta8, Delta7/8, amplicons of the respective TNFR2 splice variants.

FIG. 10 shows the splicing products for L929 murine cells using LNA SSO combinations targeted to TNFR2. L929 cells were treated with the indicated single or multiple LNA SSOs at 50 nM each and analyzed 24 hours later as described in FIG. 9.

FIG. 11 the splicing products for L929 murine cells using LNA SSO combinations targeted to TNFR1. L929 cells were treated with the indicated single or multiple LNA SSOs at 50 nM each and analyzed 24 hours later as described in FIG. 9.

FIG. 12 shows the splicing products for primary mouse hepatocytes treated with LNA SSOs. Primary mouse hepatocytes were transfected with 33 nM each final concentration of the indicated single or multiple LNA SSOs and analyzed as described in FIG. 9.

FIG. 13 graphically illustrates detection of secreted TNFR2 splice variants from L929 cells (left) and primary mouse hepatocytes (right). Cells were transfected with the indicated LNA SSOs. After 72 hours, the extracellular media was removed and analyzed by enzyme linked immunosorbant assay (ELISA) using antibodies from the Quantikine® Mouse sTNF RII ELISA kit from R and D Systems (Minneapolis, Minn.). The data are expressed as pg soluble TNFR2 per mL.

FIG. 14 shows the splicing products for primary human hepatocytes treated with LNA SSOs targeted to TNFR2. Primary human hepatocytes were transfected with the indicated LNA SSO and analyzed for splice switching by RT-PCR after 24 hours as described in FIG. 9. The PCR primers were used to amplify from Exon 5 to Exon 9, so that “Full Length” (FL) TNFR2 is represented by a 463 bp band. Transcripts lacking exon 7 (Delta Exon 7), lacking exon 8 (Delta Exon 8), and lacking both exon 7 and exon 8 (Delta exon 7/8) are represented by 385 bp, 428 bp, and 350 bp bands, respectively.

FIG. 15 shows the splicing products for intraperitoneal (i.p.) injection of LNA 3274 (top) and 3305 (bottom) in mice. LNA 3274 was injected i.p. at 25 mg/kg/day for either 4 days (4/1 and 4/10) or 10 days (10/1). Mice were sacrificed either 1 day (4/1 and 10/1) or 10 (4/10) days after the last injection and total RNA from liver was analyzed for splice switching of TNFR2 by RT-PCR. LNA 3305 was injected at the indicated dose per day for 4 days. Mice were sacrificed the next day and the livers analyzed as with 3274 treated animals.

FIG. 16 (top panel) graphically illustrates the amount of soluble TNFR2 in mouse serum 10 days after SSO treatment. Mice were injected i.p. with the indicated SSO or saline (n=5 per group) at 25 mg/kg/day for 10 days. Serum collected 4 days before injections began and the indicated number of days after the last injection. Sera was analyzed by ELISA as described in FIG. 13. At day 10, mice were sacrificed and livers were analyzed for TNFR2 splice switching by RT-PCR (bottom panel) as described in FIG. 9.

FIG. 17 graphically illustrates the amount of soluble TNFR1 in the serum after TNFR2 SSO treatment. Mouse serum from FIG. 16 was analyzed for soluble TNFR1 by ELISA using antibodies from the Quantikine® Mouse sTNF RI ELISA kit from R and D Systems (Minneapolis, Minn.).

FIG. 18 (top panel) graphically illustrates the amount of soluble TNFR2 in mouse serum 27 days after SSO treatment. Mice were treated as in FIG. 16, except that serum samples were collected until day 27 after the last injection. LNA 3083 and 3272 are control SSOs with no TNFR2 splice switching ability. At day 27, mice were sacrificed and livers were analyzed for TNFR2 splice switching by RT-PCR (bottom panel) as described in FIG. 9.

FIG. 19 graphically depicts the anti-TNF-alpha activity in serum from LNA oligonucleotide-treated mice. L929 cells were treated with either 0.1 ng/mL TNF-alpha (TNF), or TNF-alpha plus 10% serum from mice treated with the indicated oligonucleotide (see also FIG. 18). Cell viability was measured 24 hours later and normalized to untreated cells (Untreated).

FIG. 20 graphically compares the anti-TNF-alpha activity of serum from LNA oligonucleotide-treated mice to recombinant soluble TNFR2 (rsTNFR2) and to that of Enbrel® using the cell survival assay described in FIG. 19.

FIG. 21 schematically depicts the human TNFR2 structure. Relevant exons and introns are represented by boxes and lines, respectively. The signal sequence and the transmembrane region are shaded. Residues that form the boundaries of the signal sequence, the transmembrane region, and the final residue are indicated below the diagram. Exon boundaries are indicated above the diagram; if the 3′ end of an exon and the 5′ end of the following exon have the same residue number, then the splice junction is located within the codon encoding that residue.

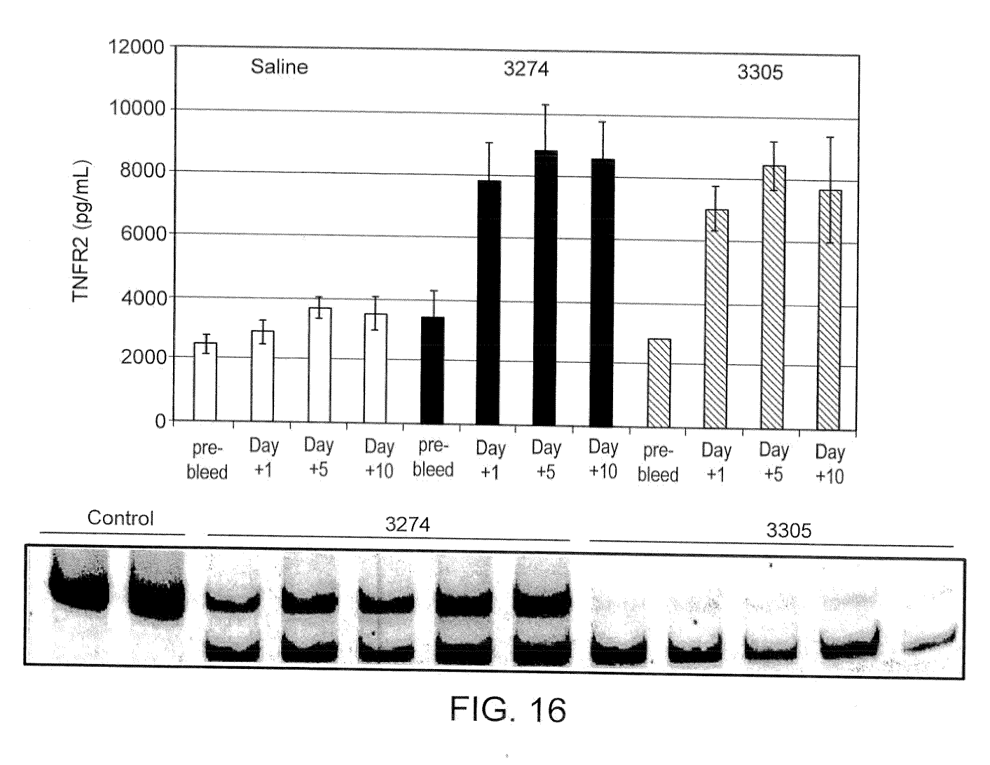

FIG. 22A graphically illustrates the amount of soluble TNFR2 from SSO treated primary human hepatocytes. The indicated SSO was transfected into primary human hepatocytes at 50 nM. After ˜48 hrs, the extracellular media was analyzed by enzyme linked immunosorbant assay (ELISA) for soluble TNFR2 using the Quantikine® Human sTNF RII ELISA kit from R&D Systems (Minneapolis, Minn.). Error bars represent the standard deviation for 3 independent experiments.

FIG. 22B Total RNA was analyzed for TNFR2 splice switching by RT-PCR using primers specific for human TNFR2. SSOs targeted to exon seven led to shifting from full length TNFR2 mRNA (FL) to TNFR2 Δ7 mRNA (Δ7). SSO 3083 is a control SSO with no TNFR2 splice switching ability.

FIG. 23 shows the splicing products of L929 cells treated with SSO 10-mers targeted to mouse TNFR2 exon 7. L929 cells were transfected with the indicated SSO concentration (50 or 100 nM), and evaluated for splice switching of TNFR2 by RT-PCR 24 hrs later. PCR primers were used to amplify from Exon 5 to Exon 9, so that “Full Length” (FL) TNFR2 is represented by a 486 bp band. Transcripts lacking exon 7 (Δ7) is represented by a 408 bp band.

FIGS. 24A and 4B show the splicing products of mice treated with SSO 10-mers targeted to mouse TNFR2 exon 7. The indicated SSOs were resuspended in saline, and injected i.p. into mice at 25 mg/kg/day for 5 days. Mice were prebled before SSO injection, and 10 days after the final SSO injection and sacrificed. At the time of sacrifice, total RNA from livers was analyzed for TNFR2 splice switching by RT-PCR. FL—full length TNFR2; Δ7—TNFR2 Δ7 (FIG. 24A). The concentration of TNFR2 Δ7 in the serum taken before (Pre) and after (Post) SSO injection was determined by ELISA using the Quantikine® Mouse sTNF RII ELISA kit from R&D Systems (Minneapolis, Minn.) (FIG. 24B). Error bars represent the standard error from 3 independent readings of the same sample.

FIG. 25 depicts the splice switching ability of SSOs of different lengths. Primary human hepatocytes were transfected with the indicated SSO and TNFR2 expression analyzed by RT-PCR (top panel) and ELISA (bottom panel) as in FIG. 2. Error bars represent the standard deviation from 2 independent experiments.

FIGS. 26A and 26B illustrate TNFR2 Δ7 mRNA induction in the livers of SSO treated mice. FIG. 26A: Total RNA from the livers of SSO 3274 treated mice were subjected to RT-PCR, and the products visualized on a 1.5% agarose gel. The sequence of the exon 6-exon 8 junction is shown in FIG. 26B.

FIGS. 27A and 27B illustrate TNFR2 Δ7 mRNA induction in SSO treated primary human hepatocytes. FIG. 27A: Total RNA from SSO 3379 treated cells were subjected to RT-PCR, and the products visualized on a 1.5% agarose gel. The sequence of the exon 6-exon 8 junction is shown in FIG. 27B.

FIGS. 28A and 28B illustrate the dose dependence of TNFR2 pre-mRNA splicing shifting by SSO 3378, 3379 and 3384. Primary human hepatocytes were transfected with 1-150 nM of the indicated SSO. After ˜48 hrs, the cells were harvested for total RNA, and the extracellular media was collected. FIG. 28A: Total RNA was analyzed for TNFR2 splice switching by RT-PCR using primers specific for human TNFR2. For each SSO, amount of splice switching is plotted as a function of SSO concentration. FIG. 28B: The concentration of soluble TNFR2 in the extracellular media was determined by ELISA and plotted as a function of SSO. Error bars represent the standard deviation for at least 2 independent experiments.

FIG. 29 graphically illustrates detection of secreted TNFR2 splice variants from L929 cells. Cells were transfected with the indicated SSOs. After 72 hrs, the extracellular media was removed and analyzed by ELISA. The data are expressed as pg soluble TNFR2 per mL.

FIG. 30 shows the splicing products for intraperitoneal (i.p.) injection of SSO 3274 (top) and 3305 (bottom) in mice. SSO 3274 was injected i.p. at 25 mg/kg/day for either 4 days (4/1 and 4/10) or 10 days (10/1). Mice were sacrificed either 1 day (4/1 and 10/1) or 10 days (4/10) after the last injection and total RNA from liver was analyzed by RT-PCR for TNFR2 splice switching. SSO 3305 was injected at the indicated dose per day for 4 days. Mice were sacrificed the next day and the livers analyzed as with 3274 treated animals.

FIG. 31A graphically illustrates the amount of soluble TNFR2 in mouse serum 10 days after SSO treatment. Mice were injected i.p. with the indicated SSO or saline (n=5 per group) at 25 mg/kg/day for 10 days. Serum was collected 4 days before injections began and on the indicated days after the last injection. Sera was analyzed by ELISA as described in FIG. 22 At day 10, mice were sacrificed and livers were analyzed for TNFR2 splice switching by RT-PCR (FIG. 31B) as described in FIG. 30.

FIG. 32A graphically illustrates the amount of soluble TNFR2 in mouse serum 27 days after SSO treatment. Mice were treated as described in FIG. 31, except that serum samples were collected until day 27 after the last injection. SSOs 3083 and 3272 are control SSOs with no TNFR2 splice switching ability. At day 27, mice were sacrificed and livers were analyzed for TNFR2 splice switching by RT-PCR (FIG. 32B) as described in FIG. 31.

FIGS. 33A and 33B graphically depict the anti-TNF-α activity in a cell-based assay using serum from SSO treated mice, where serum samples were collected 5 days (FIG. 26A) and 27 days (FIG. 26B) after SSO treatment. L929 cells were treated with either 0.1 ng/mL TNF-α, or TNF-α plus 10% serum from mice treated with the indicated SSO. Cell viability was measured 24 hrs later and normalized to untreated cells.

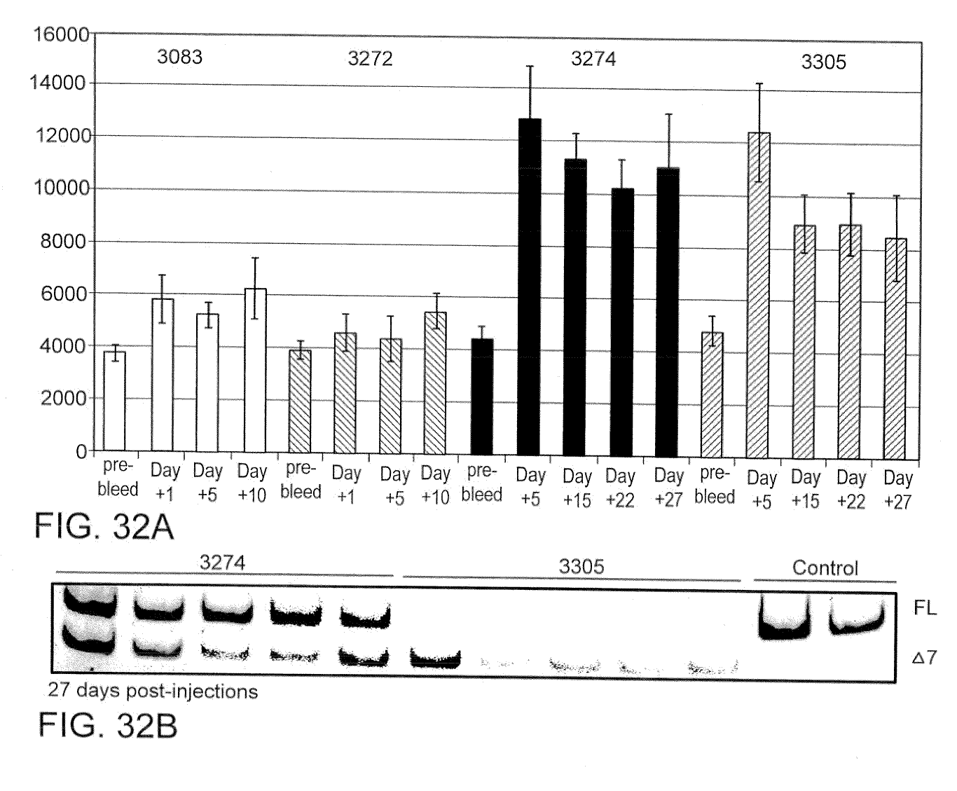

FIG. 34 graphically compares the anti-TNF-α activity of serum from the indicated SSO oligonucleotide-treated mice to recombinant soluble TNFR2 (rsTNFR2) extracellular domain from Sigma® and to Enbrel® using the cell survival assay described in FIG. 33.

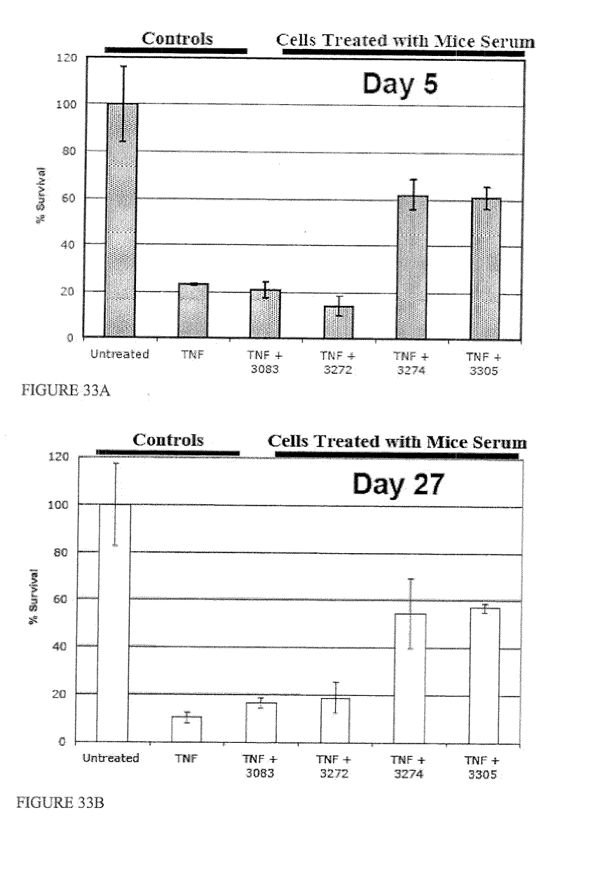

FIGS. 35A and 35B compare the stability of muTNFR2 Δ7 protein (FIG. 35A) and mRNA (FIG. 35B). Mice were injected at 25 mg/kg/day daily with either SSO 3272, SSO 3274 or SSO 3305 (n=5). Mice were bled on the indicated day after the last injection and the serum TNFR2 concentration was measured. Total RNA from mice sacrificed on the indicated day after the last injection of SSO was subjected to RT-PCR as described in FIG. 30.

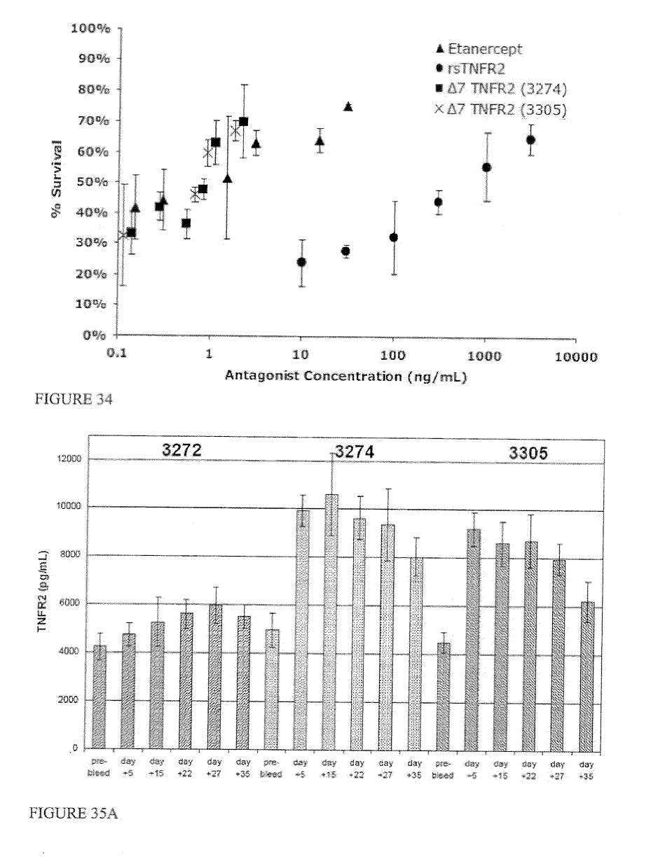

FIG. 36 plots TNFR2 Δ7 protein (dashed line) and mRNA (solid line) levels over time, as a percentage of the amount of protein or mRNA, respectively, 10 days after the last injection.

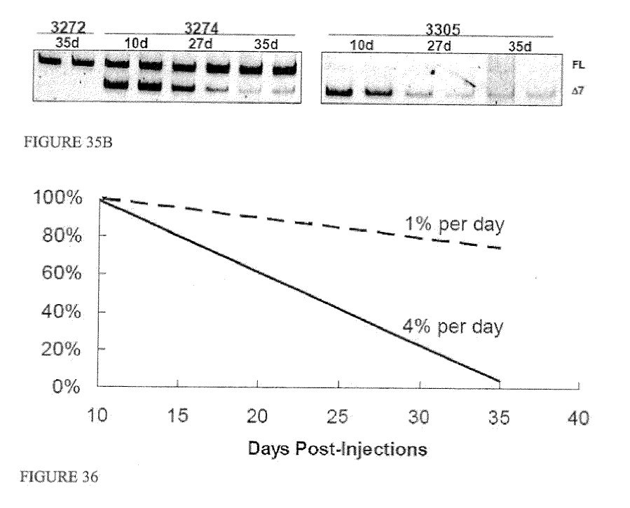

FIG. 37 graphically illustrates the dose dependant anti-TNF-α activity of TNFR2 Δ7 expressed in HeLa cells after transfection with TNFR2 Δ7 mammalian expression plasmids. HeLa cells were transfected with the indicated mouse or human TNFR2 Δ7 plasmid and extracellular media was collected after 48 hrs. The TNFR2 Δ7 concentration in the media was determined by ELISA and serial dilutions were prepared. These dilutions were assayed for anti-TNF-α activity by the L929 cytoxicity assay as in FIG. 34.

FIG. 38 shows expressed mouse (A) and human (B) TNFR2 D7 protein isolated by polyacrylamide gel electrophoresis (PAGE). HeLa cells were transfected with the indicated plasmid. After ˜48 hrs, the extracellular media was collected and concentrated, and cells were collected in RIPA lysis buffer. The proteins in the samples were separated by PAGE and a western blot was performed using a C-terminal TNFR2 primary antibody (Abeam) that recognizes both the human and mouse TNFR2 D7 proteins. Media, extracellular media samples from HeLa cells transfected with the indicated plasmid; Lysate, cell lysate from Hela cells transfected with the indicated plasmid. CM, control media from untransfected HeLa cells; CL, control cell lysates from untransfected HeLa cells. +, molecular weight markers (kDal).

FIG. 39 shows purified His-tagged human and mouse TNFR2 D7. Unconcentrated extracellular media containing the indicated TNFR2 D7 protein was prepared as in FIG. 38. Approximately 32 mL of the media was applied to a 1 mL HisPur cobalt spin column (Pierce), and bound proteins were eluted in 1 mL buffer containing 150 mM imidazole. Samples of each were analyzed by PAGE and western blot was performed as in FIG. 38. The multiple bands in lanes 1144-4 and 1319-1 represent variably glycosylated forms of TNFR2 D7.

FIG. 40 Alignment of oligomer motifs according to the invention compared against their target sequence —SEQ ID NO 1 (FIG. 40A), SEQ ID NO 2 (FIG. 40B), SEQ ID NO 3 (FIG. 40C), and SEQ ID NO 4 (FIG. 40D).

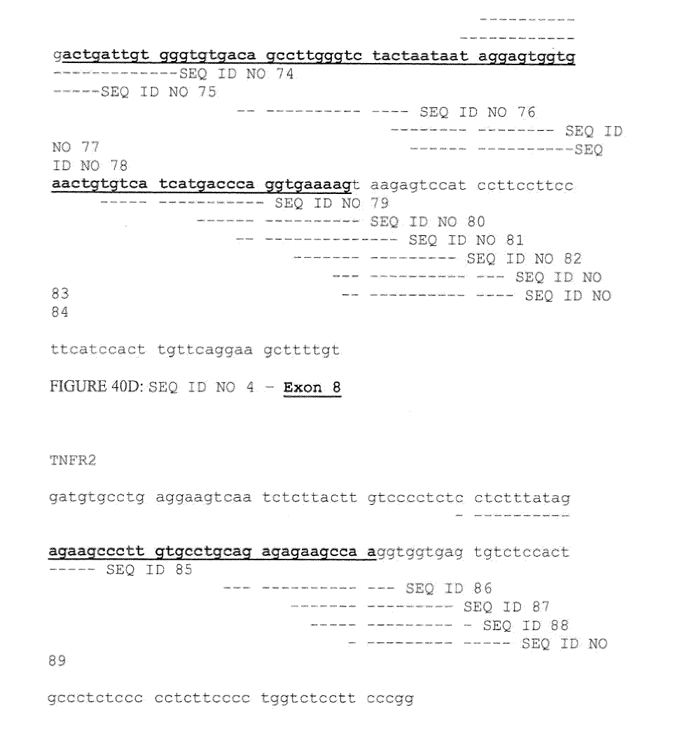

### DETAILED DESCRIPTION OF THE INVENTION

The present invention provides compositions and methods for controlling expression of TNF receptors (TNFR1 and TNFR2) and of other cytokine receptors from the TNFR superfamily by controlling the splicing of pre-mRNA that code for the said receptors. More specifically, the invention causes the increased expression of the secreted form and the decreased expression of the integral-membrane form. Furthermore, the invention can be used in the treatment of diseases associated with excessive cytokine activity.

The exon or exons that are present in the integral membrane form mRNA but are removed from the primary transcript (the “pre-mRNA”) to make a secreted form mRNA are termed the “transmembrane exons.” The invention involves nucleic acids and nucleic acid analogs that are complementary to either of the transmembrane exons and/or adjacent introns of a receptor pre-mRNA. Complementarity can be based on sequences in the sequence of pre-mRNA that spans the splice site, which would include, but is not limited to, complementarity based on sequences that span the exon-intron junction, or complementarity can be based solely on the sequence of the intron, or complementarity can be based solely on the sequence of the exon.

There are several alternative chemistries available and known to those skilled in the art. One important feature is the ability to hybridize to a target RNA without causing degradation of the target by RNase H as do 2′-deoxy oligonucleotides (“antisense oligonucleotides” hereafter “ASON”). For clarity, such compounds will be termed splice-switching oligomers (SSOs). Those skilled in the art appreciate that SSO include, but are not limited to, 2′ O-modified oligonucleotides and ribonucleosidephosphorothioates as well as peptide nucleic acids and other polymers lacking ribofuranosyl-based linkages.

One embodiment of the invention is a method of treating an inflammatory disease or condition by administering SSOs to a patient or a live subject. The SSOs that are administered alter the splicing of a pre-mRNA to produce a splice variant that encodes a stable, secreted, ligand-binding form of a receptor of the TNFR superfamily, thereby decreasing the activity of the ligand for that receptor. In another embodiment, the invention is a method of producing a stable, secreted, ligand-binding form of a receptor of the TNFR superfamily in a cell by administering SSOs to the cell.

One embodiment of the invention is a protein, either full length or mature, which can bind TNF, is encoded by a cDNA derived from a mammalian TNFR gene, and in the cDNA exon 6 is followed directly by exon 8 and as a result lacks exon 7 (“TNFR δ7”). In another embodiment, the invention is a pharmaceutical composition comprising a TNFR δ7. In a further embodiment, the invention is a method of treating an inflammatory disease or condition by administering a pharmaceutical composition comprising a TNFR δ7.

In yet another embodiment, the invention is a nucleic acid that encodes a TNFR δ7. In a further embodiment, the invention is a pharmaceutical composition comprising a nucleic acid that encodes a TNFR δ7.

In another embodiment, the invention is an expression vector comprising a nucleic acid that encodes a TNFR δ7. In a further embodiment, the invention is a method of increasing the level of a soluble TNFR in the serum of a mammal by transforming cells of the mammal with an expression vector comprising a nucleic acid that encodes a TNFR δ7.

In another embodiment, the invention is a cell transformed with an expression vector comprising a nucleic acid that encodes a TNFR δ7. In a further embodiment, the invention is a method of producing a TNFR δ7 by culturing, under conditions suitable to express the TNFR δ7, a cell transformed with an expression vector comprising a nucleic acid that encodes a TNFR δ7. In yet another embodiment, the invention is a method of treating an inflammatory disease or condition by administering an expression vector comprising a nucleic acid that encodes a TNFR δ7.

In yet another embodiment, splice-switching oligomers (SSOs) are disclosed that alter the splicing of a mammalian TNFR2 pre-mRNA to produce a mammalian TNFR2 protein, which can bind TNF and where exon 6 is followed directly by exon 8 and as a result lacks exon 7 (“TNFR2 δ7”). One embodiment of the invention is a method of treating an inflammatory disease or condition by administering SSOs to a patient or a live subject. The SSOs that are administered alter the splicing of a mammalian TNFR2 pre-mRNA to produce a TNFR2 δ7. In another embodiment, the invention is a method of producing a TNFR2 δ7 in a cell by administering SSOs to the cell.

The foregoing and other objects and aspects of the present invention are discussed in detail in the drawings herein and the specification set forth below.

### The Oligomer

In one embodiment the oligomer consists of the contiguous nucleobase sequence.

However, it is also envisaged that the oligomer may comprise of other nucleobase sequence which typically flank the contiguous nucleobase sequence at either the 5′ or 3′ end or further nucleobase sequence at both the 5′ and 3′ ends. Suitably these 5′ and or 3 ‘flanking’ regions may be 1, 2, 3, 4, 5, or 6 nucleobases in length. DNA or RNA nucleobases which are at the termini of the oligomer of the invention are expected to be cleaved from the oligomer when used in vivo by endogenous exo-nucleases—as such the inclusion of flanking DNA or RNA units may not affect the in vivo performance of the oligomer.

In one embodiment, the 3′ end of the contiguous nucleobase sequence is flanked by 1, 2 or 3 DNA or RNA units. 3′, DNA units are frequently used during solid state synthesis of oligomers.

In one embodiment, the 5′ end of the contiguous nucleobase sequence is flanked by 1, 2 or 3 DNA or RNA units.

In one embodiment the invention provides an oligomer of between 8 and 50 nucleobases in length, comprising of a contiguous nucleobase sequence which consists of between 8 and 50 nucleobases in length, wherein said contiguous nucleobase sequence is complementary to a corresponding region of contiguous nucleotides present in SEQ ID NO 1 or SEQ ID NO 2, SEQ ID NO 3, or SEQ ID NO 4 (i.e. said contiguous nucleobase sequence is present in a region (‘corresponding’—or part of) of contiguous nucleotides present in SEQ ID NO 247 or SEQ ID NO 248, SEQ ID NO 249, or SEQ ID NO 250) and wherein said contiguous nucleobase sequence does not comprise 5 or more contiguous DNA (2′-deoxyribosnucleoside) monomer units.

In one embodiment the oligomer is essentially incapable of recruiting RNAseH when formed in a duplex with a complex with a complementary mRNA molecule.

In one embodiment, the nucleotide analogues (X) are independently selected form the group consisting of: 2′-O-alkyl-RNA unit, 2′-OMe-RNA unit, 2′ MOE RNA unit, 2′-amino-DNA unit, 2′-fluoro-DNA unit, LNA unit, PNA unit, HNA unit, INA unit.

In one embodiment, the contiguous nucleobases sequence does not comprise 2′OMe ribonucleotide analogues or 2′-MOE ribonucleotide analogues.

In one embodiment, the invention provides an oligomer of between 8 and 16 nucleobases in length, comprising of a contiguous nucleobase sequence which consists of between 8 and 16 nucleobases in length, wherein said contiguous nucleobase sequence is complementary to a corresponding region of contiguous nucleotides present in SEQ ID NO 1 or SEQ ID NO 2, SEQ ID NO 3, SEQ ID NO 4 and wherein said contiguous nucleobase sequence does not comprise 5 or more contiguous DNA (2′-deoxyribosnucleoside) monomer units, wherein said contiguous nucleobases sequence comprises at least one nucleotide analogue selected from the group consisting of: beta-D-oxy LNA, thio-LNA, amino-LNA and ena-LNA.

Optionally, in the above embodiment the contiguous nucleobase sequence comprises or consists of at least one further nucleotide analogue (X).

In one embodiment, the further nucleotide analogue units, are independently selected from the group consisting of: 2′-OMe-RNA units, 2′-fluoro-DNA units, 2′-MOE RNA unit, and LNA units.

In one embodiment, the oligomer or contiguous nucleobase sequence consists of between 8 and 15 nucleobases in length, such 9, 10, 11, 12, 13 or 14 nucleobases.

In one embodiment the contiguous nucleobase sequence comprises or consists of nucleotide analogues (X).

In one embodiment the nucleotide analogues (X) are independently selected form the group consisting of: 2′-O-alkyl-RNA unit, 2′-OMe-RNA unit, 2′-amino-DNA unit, 2′-fluoro-DNA unit, LNA unit, PNA unit, HNA unit, INA unit.

In one embodiment the contiguous nucleobase sequence comprises both nucleotide analogues (X) and nucleotides (x).

In one embodiment the contiguous nucleobase sequence does not comprise a region of more than 7 consecutive nucleotide analogue units (X), such as not more than 6, not more than 5, not more than 4, not more than 3, or not more than 2 consecutive nucleotide analogue units (X).

In one embodiment the 5′ most nucleobase of the contiguous nucleobase sequence is a nucleotide analogue (X).

In one embodiment the 5′ most nucleobase of the contiguous nucleobase sequence is a nucleotide unit (x), such as a DNA (2′-deoxyribosnucleoside) monomer unit.

In one embodiment the 3′ most nucleobase of the contiguous nucleobase sequence is a nucleotide analogue (X).

In one embodiment the 3′ most nucleobase of the contiguous nucleobase sequence is a nucleotide unit (x), such as a DNA (2′-deoxyribosnucleoside) monomer unit.

In one embodiment the contiguous nucleobase sequence comprises or consists of an alternating sequence of nucleotides and nucleobases.

In one embodiment the alternating sequence of nucleotides and nucleobases is an, sequence selected from the group consisting of Xx, xX, Xxx, xXx, xxX, XXx, XxX, xXX, XXXx, XXxX, XxXX, xXXX, xxxX, xxXx, xXxx, Xxxx, XXXXx, XXXxX, XXxXX, XxXXX, xXXXX, xxxxX, xxxXx, xxXxx, xXxxx, Xxxxx, wherein said alternating sequence is optionally repeated.

In one embodiment the repeated sequence is repeated for the entire length of the contiguous nucleobase sequence, wherein, optionally the 5′ and/or 3′ repeat may be truncated.

In one embodiment the single stranded oligonucleotide comprises said at least one LNA analogue unit and at least one further nucleotide analogue unit other than LNA.

In one embodiment the single stranded oligonucleotide consists of at least one sequence X¹X²X¹ or X²X¹X², wherein X¹ is LNA and X² is a nucleotide analogue other than LNA, such as either a 2′-OMe RNA unit and 2′-fluoro DNA unit.

In one embodiment the sequence of nucleobases of the single stranded oligonucleotide consists of alternative X¹ and X² units.

In one embodiment the nucleotide analogue units, such as X, are independently selected from the group consisting of: 2′-OMe-RNA units, 2′-fluoro-DNA units, and LNA units.

In one embodiment the nucleotide analogue units (X) are LNA units.

In one embodiment the LNA units are selected from the group consisting of oxy-LNA, amino-LNA, thio-LNA, and ena-LNA.

In one embodiment the contiguous nucleobase sequence does not comprise a contiguous sub-sequence consisting of 5 or more contiguous nucleobases independently selected from DNA and LNA units, wherein the LNA units present in the contiguous sub-sequence are in the alpha-L-configuration.

In one embodiment the contiguous nucleobase sequence does not comprise a contiguous sub-sequence consisting of 5 or more contiguous nucleobases independently selected from DNA and LNA units, wherein the LNA units present in the contiguous sub-sequence are alpha-L-oxy LNA.

In one embodiment the all the LNA units are in the beta-D configuration.

In one embodiment the length of the contiguous nucleobase sequence is between 8 and 16, such as 9, 10, 11, 12, 13, 14, 15 or 16 nucleobases, in length, or between 10-14 or 11-14 or 12-14.

In one embodiment the length of the contiguous nucleobase sequence is between 8 and 15, such as 8, 9, 10, 11, 12, 13, 14, or 15 nucleobases, in length.

In one embodiment the contiguous nucleobase sequence comprises a nucleobase sequence which is complementary to a corresponding region of SEQ ID NO 1 or SEQ ID NO 3, i.e is present in a (corresponding) region of contiguous nucleotides present in SEQ ID NO 247 or SEQ ID NO 249.

In one embodiment the contiguous nucleobase sequence is complementary to a corresponding region of contiguous nucleotides present in a sequence selected from the group consisting of: 51-164 of SEQ ID NO 1, 51-79 of SEQ ID NO 2, 51-127 of SEQ ID NO 3, and 51-85 of SEQ ID NO 4.

In one embodiment the contiguous nucleobase sequence is complementary to a corresponding region of contiguous nucleotides present in a sequence selected from the group consisting of: 1-50 of SEQ ID NO 1, 165-215 of SEQ ID NO 1, 1-50 of SEQ ID NO 2, 80-130 of SEQ ID NO 2, 1-50 of SEQ ID NO 3, 128-178 of SEQ ID NO 3, 1-50 of SEQ ID NO 4, and 86-136 of SEQ ID NO 4.

In one embodiment the contiguous nucleobase sequence comprises a nucleobase sequence which is complementary to an 5′ exon/intron 3′ or 3′ intron/exon 5′ border.

In one embodiment the 5′ exon/intron 3′ or 3′ intron/exon 5′ border is selected from the group consisting of nucleobases 50-51 of SEQ ID NO 1, 164-165 of SEQ ID NO 1, 50-51 of SEQ ID NO 2, 79-80 of SEQ ID NO 2, 51-52 of SEQ ID NO 3, 129-139 of SEQ ID NO 3, 50-51 of SEQ ID NO 4, 81-82 of SEQ ID No 4.

In one embodiment the contiguous nucleobase sequence is identical to or is present in a nucleobase sequence present in a sequence selected from the group consisting of SEQ ID NO 74 to SEQ ID NO 105.

In one embodiment the contiguous nucleobase sequence is identical to or is present in a nucleobase sequence selected from the group consisting of: SEQ ID NO 74, SEQ ID NO 75, SEQ ID NO 77, SEQ ID NO 78, SEQ ID NO 80, SEQ ID NO 82, and SEQ ID NO 84.

In one embodiment the contiguous nucleobase sequence is identical to or is present in a nucleobase sequence selected from the group consisting of: SEQ ID NO 85, SEQ ID NO 86, SEQ ID NO 87, SEQ ID NO 88, and SEQ ID NO 89.

In one embodiment the oligomer is selected from the group consisting of: SEQ ID NO 74, SEQ ID NO 75, SEQ ID NO 77, SEQ ID NO 78, SEQ ID NO 80, SEQ ID NO 82, and SEQ ID NO 84.

In one embodiment the oligomer is selected from the group consisting of: SEQ ID NO 86, SEQ ID NO 87, SEQ ID NO 88, and SEQ ID NO 89.

In one embodiment the contiguous nucleobase sequence comprises a nucleobase sequence which is complementary to a region of SEQ ID No 3 selected from nucleotides: 47-49, 54-56, and 122-124.

In one embodiment the contiguous nucleobase sequence is identical to or is present in a nucleobase sequence or nucleobase sequence motif selected from the group consisting of: SEQ ID NO 130-SEQ ID No 145, SEQ ID NO 146-SEQ ID NO 161, and SEQ ID NO 162-177.

In one embodiment the contiguous nucleobase sequence is identical to or is present in a nucleobase sequence or nucleobase sequence motif selected from the group consisting of: SEQ ID NO 131-SEQ ID No 145, SEQ ID NO 147-SEQ ID NO 161, and SEQ ID NO 163-177.

In one embodiment the oligomer is selected from the group consisting of: SEQ ID NO 243, SEQ ID NO 244, SEQ ID NO 245 or SEQ ID NO 246.

In one embodiment, the contiguous nucleobase sequence is identical to or is present in a nucleobase sequence or a nucleobase motif sequence selected from the group consisting of: SEQ ID NO 131-SEQ ID No 145, SEQ ID NO 147-SEQ ID NO 161, and SEQ ID NO 163-177.

In one embodiment, the oligomer is selected from the group consisting of: SEQ ID NO 245-SEQ ID NO 246, SEQ ID NO 251-263, SEQ ID NO 264-SEQ ID NO 279, and SEQ ID NO 280-SEQ ID NO 295.

In one embodiment, said contiguous nucleobase sequence is identical to or is present in a nucleobase sequence or a nucleobase motif sequence selected from the group consisting of: SEQ ID NO 130, SEQ ID NO 146, and SEQ ID NO 162.

In one embodiment, the oligomer is selected from the group consisting of: SEQ ID NO 244, SEQ ID NO 264, and SEQ ID NO 280.

In one embodiment the oligomer comprises at least one non-nucleotide moiety covalently attached to said oligomer.

### Splice-Switching Oligomers (SSOs):

In another aspect, the present invention employs splice switching oligonucleotides or splice switching oligomers (SSOs) to control the alternative splicing of TNFR2 so that the amount of a soluble, ligand-binding form that lacks exon 7 is increased and the amount of the integral membrane form is decreased. The methods and compositions of the present invention can be used in the treatment of diseases associated with excessive tnf activity.

Accordingly, one embodiment of the invention is a method of treating an inflammatory disease or condition by administering SSOs to a patient. The SSOs that are administered alter the splicing of a pre-mRNA to produce a mammalian TNFR2 protein that lacks exon 7. In another embodiment, the invention is a method of producing a mammalian TNFR2 protein that lacks exon 7 in a cell by administering SSOs to the cell.

The length of the SSO (i.e. The number of monomers in the oligomer) is similar to an antisense oligonucleotide (ASON), typically between about 8 and 30 nucleotides. In preferred embodiments, the SSO will be between about 10 to 16 nucleotides. The invention can be practiced with SSOs of several chemistries that hybridize to RNA, but that do not activate the destruction of the RNA by RNAseH, as do conventional antisense 2′-deoxy oligonucleotides.

The invention can be practiced using 2′o modified nucleic acid oligomers, such as where the 2′O is replaced with —O—CH₃, —O—CH₂—CH₂—O—CH₃, —O—CH₂—CH₂—CH₂—NH₂, —O—CH₂—CH₂—CH₂—OH or —F, where 2′O-methyl or 2′O-methyloxyethyl is preferred. The nucleobases do not need to be linked to sugars; so-called peptide nucleic acid oligomers or morpholine-based oligomers can be used. A comparison of these different linking chemistries is found in Sazani, p. e al., 2001, nucleic acids res. 29:3695. The term splice-switching oligonucleotide is intended to cover the above forms. Those skilled in the art will appreciate the relationship between antisense oligonucleotide gapmers and SSOs. Gapmers are ASON that contain an RNAse H activating region (typically a 2′-deoxyribonucleoside phosphorothioate) which is flanked by non-activating nuclease resistant oligomers. In general, any chemistry suitable for the flanking sequences in a gapmer ASON can be used in an SSO.

The SSOs of this invention may be made through the well-known technique of solid phase synthesis. Any other means for such synthesis known in the art may additionally or alternatively be used. It is well known to use similar techniques to prepare oligonucleotides such as the phosphorothioates and alkylated derivatives.

The bases of the SSO may be the conventional cytosine, guanine, adenine and uracil or thymidine. Alternatively, modified bases can be used. Of particular interest are modified bases that increase binding affinity. One non-limiting example of preferred modified bases are the so-called g-clamp or 9-(aminoethoxy)phenoxazine nucleotides, cytosine analogues that form 4 hydrogen bonds with guanosine. (Flanagan, W. M., et al., 1999, proc. Natl. Acad. Sci. 96:3513; Holmes, S. C., 2003, Nucleic Acids Res. 31:2759). Specific examples of other bases include, but are not limited to, 5-methylcytosine (^{me}C), isocytosine, pseudoisocytosine, 5-bromouracil, 5-propynyluracil, 5-propyny-6,5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine.

When LNA nucleotides are employed in an SSO it is preferred that non-LNA nucleotides also be present. LNA nucleotides have such high affinities of hybridization that there can be significant non-specific binding, which may reduce the effective concentration of the free-SSO. When LNA nucleotides are used they may be alternated conveniently with 2′-deoxynucleotides.

Alternating nucleotides, alternating dinucleotides or mixed patterns, e.g., LDLDLD or LLDLLD or LDDLDD can be used. For example in one embodiment, contains a sequence of nucleotides selected from the group consisting of: LDLDDLLDDLDLDLL, LDLDLLLDDLLLDLL, LMLMMLLMMLMLMLL, LMLMLLLMMLLLMLL, LFLFFLLFFLFLFLL, LFLFLLLFFLLLFLL, LDDLDDLDDL, DLDDLDDLDD, DDLDDLDDLD, LMMLMMLMML, MLMMLMMLMM, MMLMMLMMLM, LFFLFFLFFL, FLFFLFFLFF, FFLFFLFFLF, DLDLDLDLDL, LDLDLDLDL, MLMLMLMLML, LMLMLMLML, FLFLFLFLFL, LFLFLFLFL, where L is a LNA unit, D is a DNA unit, M is 2′Moe, F is 2′fluoro.

When 2′-deoxynucleotides or 2′-deoxynucleoside phosphorothioates are mixed with LNA nucleotides it is important to avoid RNAse H activation. It is expected that between about one third and two thirds of the LNA nucleotides of an SSO will be suitable. When affinity-enhancing modifications are used, including but not limited to LNA or g-clamp nucleotides, the skilled person recognizes it can be necessary to increase the proportion of such affinity-enhancing modifications.

Numerous alternative chemistries which do not activate RNAse H are available. For example, suitable SSOs can be oligonucleotides wherein at least one of the internucleotide bridging phosphate residues is a modified phosphate, such as methyl phosphonate, methyl phosphonothioate, phosphoromorpholidate, phosphoropiperazidate, and phosphoroamidate. For example, every other one of the internucleotide bridging phosphate residues may be modified as described. In another non-limiting example, such SSO are oligonucleotides wherein at least one of the nucleotides contains a 2′ lower alkyl moiety (e.g., c₁-c₄, linear or branched, saturated or unsaturated alkyl, such as methyl, ethyl, ethenyl, propyl, 1-propenyl, 2-propenyl, and isopropyl). For example, every other one of the nucleotides may be modified as described. (see references in U.S. Pat. No. 5,976,879 col. 4). For in vivo use, phosphorothioate linkages are preferred.

The length of the SSO will be from about 8 to about 30 bases in length. Those skilled in the art appreciate that when affinity-increasing chemical modifications are used, the SSO can be shorter and still retain specificity. Those skilled in the art will further appreciate that an upper limit on the size of the SSO is imposed by the need to maintain specific recognition of the target sequence, and to avoid secondary-structure forming self hybridization of the SSO and by the limitations of gaining cell entry. These limitations imply that an SSO of increasing length (above and beyond a certain length which will depend on the affinity of the SSO) will be more frequently found to be less specific, inactive or poorly active.

SSOs of the invention include, but are not limited to, modifications of the SSO involving chemically linking to the SSO one or more moieties or conjugates which enhance the activity, cellular distribution or cellular uptake of the SSO. Such moieties include, but are not limited to, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

It is not necessary for all positions in a given SSO to be uniformly modified, and in fact more than one of the aforementioned modifications may be incorporated in a single compound or even at a single nucleoside within an SSO.

The SSOs may be admixed, encapsulated, conjugated, or otherwise associated with other molecules, molecule structures, or mixtures of compounds, as for example liposomes, receptor targeted molecules, oral, rectal, topical or other formulation, for assisting in uptake, distribution, and/or absorption.

Those skilled in the art appreciate that cellular differentiation includes, but is not limited to, differentiation of the spliceosome. Accordingly, the activity of any particular SSO can depend upon the cell type into which they are introduced. For example, SSOs which are effective in one cell type may be ineffective in another cell type.

The methods, oligonucleotides, and formulations of the present invention are also useful as in vitro or in vitro tools to examine splicing in human or animal genes. Such methods can be carried out by the procedures described herein, or modifications thereof which will be apparent to skilled persons.

The SSOs disclosed herein can be used to treat any condition in which the medical practitioner intends to limit the effect of tnf or the signalling pathway activated by tnf. In particular, the invention can be used to treat an inflammatory disease. In one embodiment, the condition is an inflammatory systemic disease, e.g., rheumatoid arthritis or psoriatic arthritis. In another embodiment, the disease is an inflammatory liver disease. Examples of inflammatory liver diseases include, but are not limited to, hepatitis associated with the hepatitis a, b, or c viruses, alcoholic liver disease, and non-alcoholic steatosis. In yet another embodiment, the inflammatory disease is a skin condition such as psoriasis.

### RNAseH Recruitment

The oligomer according to the invention does not mediate RNAseH based cleavage of a complementary single stranded RNA molecule. A stretch of at least 5 consecutive DNA nucleobases are required for an oligonucleotide to be effective in recruitment of RNAseH.

EP 1 222 309 provides in vitro methods for determining RNaseH activity, which may be used to determine the ability to recruit RNaseH. A compound is deemed capable of recruiting RNase H if; when provided with the complementary RNA target, it has an initial rate, as measured in pmol/l/min, of at least 1%, such as at least 5%, such as at least 10% or less than 20% of the equivalent DNA only oligonucleotide, with no 2′ substitutions, with phosphorothiote linkage groups between all nucleotides in the oligonucleotide, using the methodology provided by Example 91-95 of EP 1 222 309.

A compound is deemed essentially incapable of recruiting RNaseH if, when provided with the complementary RNA target, and RNaseH, the RNaseH initial rate, as measured in pmol/l/min, is less than 20% such as less than 10% such as less than 5%, or preferably less than 1%, (or even less than 0.1%) of the initial rate determined using the equivalent DNA only oligonucleotide, with no 2′ substitutions, with phosphorothiote linkage groups between all nucleotides in the oligonucleotide, using the methodology provided by Example 91-95 of EP 1222 309.

### Nucleotide Analogues

It will be recognised that when referring to a preferred nucleotide sequence motif or nucleotide sequence, which consists of only nucleotides, the oligomers of the invention which are defined by that sequence may comprise a corresponding nucleotide analogues in place of one or more of the nucleotides present in said sequence, such as LNA units or other nucleotide analogues, which raise the duplex stability/Tₘ of the oligomer/target duplex (i.e. affinity enhancing nucleotide analogues).

Furthermore, the nucleotide analogues may enhance the stability of the oligomer in vivo.

Incorporation of affinity-enhancing nucleotide analogues in the oligomer, such as LNA or 2′-substituted sugars, can allow the size of the specifically binding oligomer to be reduced, and may also reduce the upper limit to the size of the oligomer before non-specific or aberrant binding takes place.

Suitably, when the nucleobase sequence of the oligomer, or the contiguous nucleobase sequence, is not fully complementary to the corresponding region of the TNFR target sequence, in one embodiment, when the oligomer comprises affinity enhancing nucleotide analogues, such nucleotide analogues form a complement with their corresponding nucleotide in the TNFR target.

The oligomer may thus comprise or consist of a simple sequence of natural nucleotides—preferably 2′-deoxynucleotides (referred to here generally as “DNA”), but also possibly ribonucleotides (referred to here generally as “RNA”)—or it could comprise one or more (and possibly consist completely of) nucleotide “analogues”.

Nucleotide “analogues” are variants of natural DNA or RNA nucleotides by virtue of modifications in the sugar and/or base and/or phosphate portions. The term “nucleobase” will be used to encompass natural (DNA- or RNA-type) nucleotides as well as such “analogues” thereof. Analogues could in principle be merely “silent” or “equivalent” to the natural nucleotides in the context of the oligonucleotide, i.e. have no functional effect on the way the oligonucleotide works to inhibit beta-catenin expression. Such “equivalent” analogues may nevertheless be useful it; for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell.

Examples of such modification of the nucleotide include modifying the sugar moiety to provide a 2′-substituent group or to produce a bridged (locked nucleic acid) structure which enhances binding affinity and probably also provides some increased nuclease resistance; modifying the internucleotide linkage from its normal phosphodiester to one that is more resistant to nuclease attack, such as phosphorothioate or boranophosphate.

A preferred nucleotide analogue is LNA, such as beta-D-oxy-LNA, alpha-L-oxy-LNA, beta-D-amino-LNA and beta-D-thio-LNA, most preferred beta-D-oxy-LNA.

In some embodiments, the oligomer comprises from 3-8 nucleotide analogues, e.g. 6 or 7 nucleotide analogues. In the by far most preferred embodiments, at least one of said nucleotide analogues is a locked nucleic acid (LNA); for example at least 3 or at least 4, or at least 5, or at least 6, or at least 7, or 8, of the nucleotide analogues may be LNA. In some embodiments all the nucleotides analogues may be LNA.

In some embodiments the nucleotide analogues present within the oligomer of the invention are independently selected from, for example: 2′-O-alkyl-RNA units, 2′-amino-DNA units, 2′-fluoro-DNA units, LNA units, arabino nucleic acid (ANA) units, 2′-fluoro-ANA units, HNA units, INA (intercalating nucleic acid) units and 2′MOE units.

In one embodiment the nucleotide analogue is 2′-MOE, i.e. 2′O-2-methoxyethyl RNA.

In one embodiment the nucleotide analogue is 2′-MOE, i.e. 2′O-2-methoxyethyl RNA. Therefore in one embodiment X² or M as referred to in nucleobases motifs herein may be 2′-MOE.

2′-O-methoxyethyl-RNA (2′MOE), 2′-fluoro-DNA monomers and LNA are preferred nucleotide analogues, and as such the oligonucleotide of the invention may comprise nucleotide analogues which are independently selected from these three types of analogue, or may comprise only one type of analogue selected from the three types.

Preferably, the oligomer according to the invention comprises at least one Locked Nucleic Acid (LNA) unit, such as 1, 2, 3, 4, 5, 6, 7, or 8 LNA units, preferably between 4 to 8 LNA units, most preferably 4, 5 or 6 LNA units. Suitably, the oligomer may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

In one embodiment of the invention, the oligomer may comprise both LNA and DNA units. Preferably the combined total of LNA and DNA units is 8-24, such as 8-15 or 10-25, or 10-20, or 12-16.

In one embodiment of the invention, the nucleobase sequence of the oligomer, such as the contiguous nucleobase sequence consists of at least one LNA and the remaining nucleobase units are DNA units.

In some embodiments of oligomer according to the invention, such as an antisense oligonucleotide which comprises LNA, all LNA C units are 5′methyl-Cytosine. In some embodiments, all the nucleotide analogues are LNA.

In most preferred embodiments the oligomer comprises only LNA nucleotide analogues and nucleotides (RNA or DNA, most preferably DNA nucleotides, optionally with modified internucleobase linkages such as phosphorothioate).

In some embodiments at least one of said nucleotide analogues is 2′-MOE-RNA, such as 2, 3, 4, 5, 6, 7 or 8 2′-MOE-RNA nucleobase units.

In some embodiments at least one of said nucleotide analogues is 2′-fluoro DNA, such as 2, 3, 4, 5, 6, 7 or 8 2′-fluoro-DNA nucleobase units.

Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1:

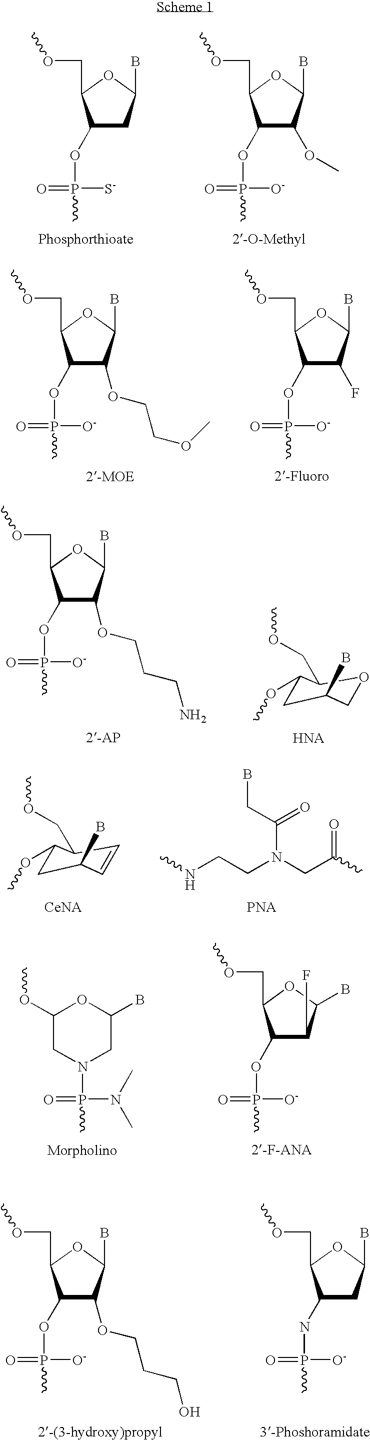

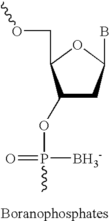

The term “LNA” refers to a bicyclic nucleotide analogue, known as “Locked Nucleic Acid”. It may refer to an LNA monomer, or, when used in the context of an “LNA oligonucleotide” refers to an oligonucleotide containing one or more such bicyclic nucleotide analogues.

A particularly preferred chemistry is provided by locked nucleic acids (LNA) (Koshkin, A. A., et al., 1998, Tetrahedron 54:3607; Obika, S., et al., 1998, Tetrahedron Lett. 39:5401). As used herein, the terms “LNA unit”, “LNA monomer”, “LNA residue”, “locked nucleic acid unit”. “locked nucleic acid monomer” or “locked nucleic acid residue”, refer to a bicyclic nucleoside analogue. LNA units and methods of their synthesis are described in inter alia WO 99/14226, WO 00/56746, WO 00/56748, WO 01/25248, WO 02/28875, WO 03/006475 and WO 03/095467. The LNA unit may also be defined with respect to its chemical formula. Thus, an “LNA unit”, as used herein, has the chemical structure shown in Formula I below:

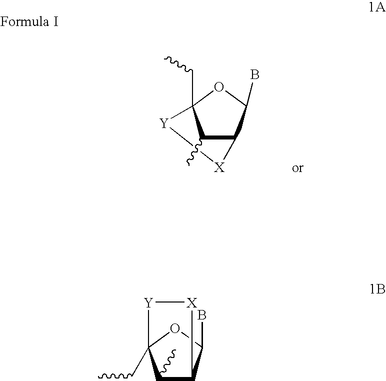

wherein,
X is selected from the group consisting of O, S and NRH, where R is H or C₁-C₄-alkyl;
Y is (—CH₂)ᵣ, where r is an integer of 1-4; and
B is a base of natural or non-natural origin as described above.

In a preferred embodiment, r is 1 or 2, and in a more preferred embodiment r is 1

The LNA used in the oligonucleotide compounds of the invention preferably has the structure of the general formula

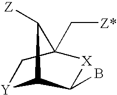

where X and Y are independently selected among the groups —O—,
—S—, —N(H)—, N(R)—, —CH₂— or —CH— (if part of a double bond),
—CH₂—O—, —CH₂—S—, —CH₂—N(H)—, —CH₂—N(R)—, —CH₂—CH₂— or —CH₂—CH— (if part of a double bond), —CH—CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligomer of the invention comprises at least one LNA unit according any of the formulas

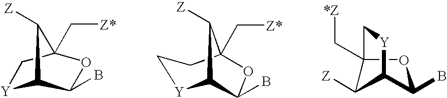

wherein Y is —O—, —S—, —NH—, or N(R^{H}); Z and Z* are independently selected among an internucleoside linkage, a terminal group or a protecting group; B constitutes a natural or non-natural nucleotide base moiety, and R^{H} is selected from hydrogen and C₁₋₄-alkyl.

Preferably, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

Specifically preferred LNA units are shown in scheme 2:

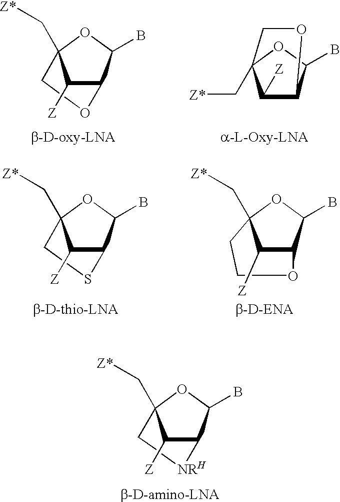

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

In a preferred embodiment LNA is selected from beta-D-oxy-LNA, alpha-L-oxy-LNA, beta-D-amino-LNA and beta-D-thio-LNA, in particular beta-D-oxy-LNA.

Preferably, the oligomer according to the invention comprises at least one nucleotide analogue, such as Locked Nucleic Acid (LNA) unit, such as 1, 2, 3, 4, 5, 6, 7, 8, 9, or 10 nucleotide analogues, such as Locked Nucleic Acid (LNA) units, preferably between 3 to 9 nucleotide analogues, such as LNA units, such as 4-8, nucleotide analogues, such as LNA units, such as 6-9 nucleotide analogues, such as LNA units, preferably 6, 7 or 8 nucleotide analogues, such as LNA units.

The oligomer according to the invention, such as an antisense oligonucleotide, may comprises of 1, 2, 3, 4, 5, 6, 7, 8, 9, 10, 11, 12, 13, 14 or 15 nucleotide analogues, such as LNA units, in particular 3, 4, 5, 6, 7, 8, 9 or 10 nucleotide analogues, such as LNA units, such as between 1 and 10 nucleotide analogues, such as LNA units such as between 2 and 8 nucleotide analogues such as LNA units.

Preferably the LNA units comprise at least one beta-D-oxy-LNA unit(s) such as 2, 3, 4, 5, 6, 7, 8, 9, or 10 beta-D-oxy-LNA units.

The oligomer of the invention, such as the antisense oligonucleotide, may comprise more than one type of LNA unit. Suitably, the compound may comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

Preferably, the oligomer, such as an antisense oligonucleotide, may comprise or consist of both nucleotide analogues, such as LNA units, and DNA units.

LNA and DNA are preferred, but MOE, 2′-O-Me, and other 2′-substituted analogues and RNA could also be used.

Preferred DNA analogues includes DNA analogues where the 2′-H group is substituted with a substitution other than —OH (RNA) e.g. by substitution with —O—CH3, —O—CH2-CH2-O—CH3, —O—CH2-CH2-CH2-NH2, —O—CH2-CH2-CH2-OH or —F.

Preferred RNA analogues includes RNA analogues which have been modified in its 2′-OH group, e.g. by substitution with a group other than —H (DNA), for example —O—CH3, —O—CH2-CH2-O—CH3, —O—CH2-CH2-CH2-NH2, —O—CH2-CH2-CH2-OH or —F.

In one embodiment the nucleotide analogue is “ENA”.

In one embodiment, the oligomer of the invention does not comprise any RNA units.

High affinity nucleotide analogues are nucleotide analogues which result in oligonucleotide which has a higher thermal duplex stability with a complementary RNA nucleotide than the binding affinity of an equivalent DNA nucleotide. This is typically determined by measuring the Tₘ.

Nucleotide analogues which increase the Tₘ of the oligomer/target nucleic acid target, as compared to the equivalent nucleotide are preferred (affinity enhancing nucleotide analogues). The oligomers may suitably be capable of hybridising against the target nucleic acid, such as a TNFR mRNA, to form a duplex with a Tₘ of at least 30° C., such as 37° C., such as at least 40° C., at least 50° C., at least 55° C., or at least 60° C. In one aspect, for example, the Tₘ is between 30° C. and 80° C., such as between 40° C. and 70° C.

In one embodiment at least 30%, such as at least 33%, such as at least 40%, such as at least 50%, such as at least 60%, such as at least 66%, such as at least 70%, such as at least 80%, such as at least 90% of the nucleobases of the oligomer of the invention are nucleotide analogues nucleobases, such as LNA. In one embodiment, all of the nucleobases of the oligomer of the invention are nucleotide analogues nucleobases, such as LNA.

It will be recognized that for shorter oligonucleotides it may be necessary to increase the proportion of (high affinity) nucleotide analogues, such as LNA.

The term “oligonucleotide” (or simply “oligo”) which is used interchangeably with the term “oligomer” refers, in the context of the present invention, to a molecule formed by covalent linkage of two or more nucleobases. When used in the context of the oligonucleotide of the invention (also referred to the single stranded oligonucleotide), the term “oligonucleotide” may have, in one embodiment, for example between 8-26 nucleobases, such as between 12 to 26 nucleobases. In a preferable embodiment, as detailed herein, the oligonucleotide of the invention has a length of between 10-16 nucleobases or 8-15 nucleobases.

### Variation of the Length of the Oligomer

The length of the oligonucleotides of the invention may vary. Indeed it is considered advantageous to have short oligonucleotides, such as between 10-17 or 10-15 nucleobases.

In such an embodiment, the oligonucleotide of the invention may have a length of 10, 11, 12, 13, 14, 15, or 16 nucleobases.

In one embodiment, the oligonucleotide according to the present has a length of from 8 to 24 nucleotides, such as 10 to 24, between 12 to 24 nucleotides, such as a length of 8, 9, 10, 11, 12, 13, 14, 15, 16, 17, 18, 19, 20, 21, 22, 23 or 24 nucleotides, preferably a length of from 10-22, such as between 12 to 22 nucleotides, such as a length of 10, 11, 12, 13, 14, 15, 16, 17, 18, 19, 20, 21 or 22 nucleotides, more preferably a length of from 10-20, such as between 12 to 20 nucleotides, such as a length of 10, 11, 12, 13, 14, 15, 16, 17, 18, 19 or 20 nucleotides, even more preferably a length of from 10 to 19, such as between 12 to 19 nucleotides, such as a length of 10, 11, 12, 13, 14, 15, 16, 17, 18 or 19 nucleotides, e.g. a length of from 10 to 18, such as between 12 to 18 nucleotides, such as a length of 10, 11, 12, 13, 14, 15, 16, 17 or 18 nucleotides, more preferably a length of from 10-17, such as from 12 to 17 nucleotides, such as a length of 10, 11, 12, 13, 14, 15, 16 or 17 nucleotides, most preferably a length of from 10 to 16, such as between 12 to 16 nucleotides, such as a length of 10, 11, 12, 13, 14, 15 or 16 nucleotides.

### Internucleoside Linkage Group

The term “internucleoside linkage group” is intended to mean a group capable of covalently coupling together two nucleobases, such as between DNA units, between DNA units and nucleotide analogues, between two non-LNA units, between a non-LNA unit and an LNA unit, and between two LNA units, etc. Preferred examples include phosphate, phosphodiester groups and phosphorothioate groups.

The internucleoside linkage may be selected form the group consisting of: —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(RH)—O—, O—PO(OCH3)-O—, —O—PO(NRH)—O—, —O—PO(OCH2CH2S—R)—O—, —O—PO(BH 3)-O—, —O—PO(NHRH)—O—, —O—P(O)₂—NRH—, —NRH—P(O)₂—O—, —NRH—CO—O—, —NRH—CO—NRH—, and/or the internucleoside linkage may be selected form the group consisting of: —O—CO—O—, —O—CO—NRH—, —NRH—CO—CH2-, —O—CH2-CO—NRH—, —O—CH2-CH2-NRH—, —CO—NRH—CH2-, —CH2-NRH—CO—, —O—CH2-CH2-S—, —S—CH2-CH2-O—, —S—CH2-CH2-S—, —CH2-SO2-CH2-, —CH2-CO—NRH—, —O—CH2-CH2-NRH—CO—, CH2-NCH3-O—CH2-, where RH is selected from hydrogen and C₁₋₄-alkyl. Suitably, in some embodiments, sulphur (S) containing internucleoside linkages as provided above may be preferred.

### Modification of the Internucleoside Linkage Group

Typical internucleoside linkage groups in oligonucleotides are phosphate groups, but these may be replaced by internucleoside linkage groups differing from phosphate. In a further interesting embodiment of the invention, the oligonucleotide of the invention is modified in its internucleoside linkage group structure, i.e. the modified oligonucleotide comprises an internucleoside linkage group which differs from phosphate. Accordingly, in a preferred embodiment, the oligonucleotide according to the present invention comprises at least one internucleoside linkage group which differs from phosphate.

Specific examples of internucleoside linkage groups which differ from phosphate

(—O—P(O)₂—O—) include —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(RH)—O—, O—PO(OCH3)-O—, —O—PO(NRH)—O—, —O—PO(OCH2CH2S—R)—O—, —O—PO(BH3)-O—, —O—PO(NHRH)—O—, —O—P(O)₂—NRH—, —NRH—P(O)₂—O—, —NRH—CO—O—, —NRH—CO—NRH—, —O—CO—O—, —O—CO—NRH—, —NRH—CO—CH2-, —O—CH2-CO—NRH—, —O—CH2-CH2-NRH—, —CO—NRH—CH2-, —CH2-NRH—CO—, —O—CH2-CH2-S—, —S—CH2-CH2-O—, —S—CH2-CH2-S—, —CH2-SO2-CH2-, —CH2-CO—NRH—, —O—CH2-CH2-NRH—CO —, —CH2-NCH3-O—CH2-, where RH is hydrogen or C₁₋₄-alkyl.

When the internucleoside linkage group is modified, the internucleoside linkage group is preferably a phosphorothioate group (—O—P(O,S)—O—). In a preferred embodiment, all internucleoside linkage groups of the oligonucleotides according to the present invention are phosphorothioate.

It is preferable for most therapeutic uses that the oligonucleotide is fully phosphorothiolated—the exception being for therapeutic oligonucleotides for use in the CNS, such as in the brain or spine where phosphorothioation can be toxic, and due to the absence of nucleases, phosphodiester bonds may be used, even between consecutive DNA units.

In one embodiment, the oligomer comprises alternating LNA and DNA units (Xx) or (xX).

In one embodiment, the oligomer comprises a motif of alternating LNA followed by 2 DNA units (Xxx), xXx or xxX.

In one embodiment, at least one of the DNA or non-LNA nucleotide analogue units are replaced with a LNA nucleobase in a position selected from the positions identified as LNA nucleobase units in any one of the embodiments referred to above.

In one embodiment, “X” donates an LNA unit.

In one embodiment, the oligomer comprises at least 3 nucleotide analogue units, such as at least 4 nucleotide analogue units, such as at least 5 nucleotide analogue units, such as at least 6 nucleotide analogue units, such as at least 7 nucleotide analogue units, such as at least 8 nucleotide analogue units, such as at least 9 nucleotide analogue units, such as at least 10, such as at least 11, such as at least 12 nucleotide analogue units.

In one embodiment, the oligomer comprises at least 3 LNA units, such as at least 4 LNA units, such as at least 5 LNA units, such as at least 6 LNA units, such as at least 7 LNA units, such as at least 8 LNA units, such as at least 9 LNA units, such as at least 10 LNA units, such as at least 11 LNA units, such as at least 12 LNA units.

In one embodiment wherein at least one of the nucleotide analogues, such as LNA units, is either cytosine or guanine, such as between 1-10 of the of the nucleotide analogues, such as LNA units, is either cytosine or guanine, such as 2, 3, 4, 5, 6, 7, 8, or 9 of the of the nucleotide analogues, such as LNA units, is either cytosine or guanine.

In one embodiment at least two of the nucleotide analogues such as LNA units is either cytosine or guanine. In one embodiment at least three of the nucleotide analogues such as LNA units is either cytosine or guanine. In one embodiment at least four of the nucleotide analogues such as LNA units is either cytosine or guanine. In one embodiment at least five of the nucleotide analogues such as LNA units is either cytosine or guanine. In one embodiment at least six of the nucleotide analogues such as LNA units is either cytosine or guanine. In one embodiment at least seven of the nucleotide analogues such as LNA units is either cytosine or guanine. In one embodiment at least eight of the nucleotide analogues such as LNA units is either cytosine or guanine.

In a preferred embodiment the nucleotide analogues have a higher thermal duplex stability a complementary RNA nucleotide than the binding affinity of an equivalent DNA nucleotide to said complementary RNA nucleotide.

In one embodiment, the nucleotide analogues confer enhanced serum stability to the single stranded oligonucleotide.

### Further Designs for Oligomers of the Invention

In one embodiment, the first nucleobase of the oligomer according to the invention, counting from the 3′ end, is a nucleotide analogue, such as an LNA unit.

In one embodiment, the second nucleobase of the oligomer according to the invention, counting from the 3′ end, is a nucleotide analogue, such as an LNA unit.

In one embodiment, x″ denotes a DNA unit.

In one embodiment, the oligomer comprises a nucleotide analogue unit, such as an LNA unit, at the 5′ end.

In one embodiment, the nucleotide analogue units, such as X, are independently selected form the group consisting of: 2′-O-alkyl-RNA unit, 2′-OMe-RNA unit, 2′-amino-DNA unit, 2′-fluoro-DNA unit, 2′-MOE-RNA unit, LNA unit, PNA unit, HNA unit, INA unit.

In one embodiment, all the nucleobases of the oligomer of the invention are nucleotide analogue units.

In one embodiment, the nucleotide analogue units, such as X, are independently selected form the group consisting of: 2′-OMe-RNA units, 2′-fluoro-DNA units, and LNA units,

In one embodiment, the oligomer comprises said at least one LNA analogue unit and at least one further nucleotide analogue unit other than LNA.

In one embodiment, the non-LNA nucleotide analogue unit or units are independently selected from 2′-OMe RNA units and 2′-fluoro DNA units.

In one embodiment, the oligomer consists of at least one sequence X¹X²X¹ or X²X¹X², wherein X¹ is LNA and X² is either a 2′-OMe RNA unit and 2′-fluoro DNA unit.

In one embodiment, the sequence of nucleobases of the oligomer consists of alternative X¹ and X² units.

In one embodiment, the oligomer according to the invention does not comprise a region of more than 5 consecutive DNA nucleotide units. In one embodiment, the oligomer according to the invention does not comprise a region of more than 6 consecutive DNA nucleotide units. In one embodiment, the oligomer according to the invention does not comprise a region of more than 7 consecutive DNA nucleotide units. In one embodiment, the oligomer according to the invention does not comprise a region of more than 8 consecutive DNA nucleotide units. In one embodiment, the oligomer according to the invention does not comprise a region of more than 3 consecutive DNA nucleotide units. In one embodiment, the oligomer according to the invention does not comprise a region of more than 2 consecutive DNA nucleotide units.

In one embodiment, the oligomer comprises at least region consisting of at least two consecutive nucleotide analogue units, such as at least two consecutive LNA units.

In one embodiment, the oligomer comprises at least region consisting of at least three consecutive nucleotide analogue units, such as at least three consecutive LNA units.

In one embodiment, the oligomer of the invention does not comprise a region of more than 7 consecutive nucleotide analogue Units, such as LNA units. In one embodiment, the oligomer of the invention does not comprise a region of more than 6 consecutive nucleotide analogue units, such as LNA units. In one embodiment, the oligomer of the invention does not comprise a region of more than 5 consecutive nucleotide analogue units, such as LNA units. In one embodiment, the oligomer of the invention does not comprise a region of more than 4 consecutive nucleotide analogue units, such as LNA units. In one embodiment, the oligomer of the invention does not comprise a region of more than 3 consecutive nucleotide analogue units, such as LNA units. In one embodiment, the oligomer of the invention does not comprise a region of more than 2 consecutive nucleotide analogue units, such as LNA units.

In one embodiment, the oligonucleotide of the invention comprises at least 50%, such as 55%, 60%, 65%, 70%, 75%, 80%, 85%, 90%, 95% or such as 100% of the nucleobase units of the oligomer are (preferably high affinity) nucleotide analogues, such as a Locked Nucleic Acid (LNA) nucleobase unit,

Table 3 and 4 below provides non-limiting examples of short microRNA sequences that could advantageously be targeted with an oligonucleotide of the present invention.

The oligonucleotides according to the invention may, in one embodiment, have a sequence of nucleobases 5′-3′ selected form the group consisting of the following motifs:

| | |
|---|---|
| LxLxxLLxxLL | |
| LxLxLLLxxLL | |
| LxxLxxLxxL | |
| xLxxLxxLxx | ‘Every third’ |
| xxLxxLxxLx | ‘Every third’ |
| xLxLxLxLxL | ‘Every second’ |
| LxLxLxLxL | ‘Every second’ |
| LdLddLLddLL | |
| LdLdLLLddLLL | |
| LMLMMLLMMLL | |
| LMLMLLLMMLL | |
| LFLFFLLFFLL | |
| LFLFLLLFFLLL | |
| LLLLLL | |
| LLLLLLL | |
| LLLLLLLL | |
| LLLLLLLLL | |
| LLLLLLLLLL | |
| LLLLLLLLLLL | |
| LLLLLLLLLLLL | |
| LMMLMMLMML | |
| MLMMLMMLMM | ‘Every third’ |
| MMLMMLMMLM | ‘Every third’ |
| LFFLFFLFFL | ‘Every third’ |
| FLFFLFFLFF | ‘Every third’ |
| FFLFFLFFLF | ‘Every third’ |
| dLdLdLdLdL | ‘Every second’ |
| LdLdLdLdL | ‘Every second’ |
| MLMLMLMLML | ‘Every second’ |
| LMLMLMLML | ‘Every second’ |
| FLFLFLFLFL | ‘Every second’ |
| LFLFLFLFL | ‘Every second’ |
| LdLddLLddLdLdLL | |
| LdLdLLLddLLLdLL | |
| LMLMMLLMMLMLMLL | |
| LMLMLLLMMLLLMLL | |
| LFLFFLLFFLFLFLL | |
| LFLFLLLFFLLLFLL | |
| LddLddLddL(d)(d)(L)(d)(d)(L)(d) | |
| dLddLddLdd(L)(d)(d)(L)(d)(d)(L) | |
| ddLddLddLd(d)(L)(d)(d)(L)(d)(d) | |
| LMMLMMLMML(M)(M)(L)(M)(M)(L)(M) | |
| MLMMLMMLMM(L)(M)(M)(L)(M)(M)(L) | |
| MMLMMLMMLM(M)(L)(M)(M)(L)(M)(M) | |
| LFFLFFLFFL(F)(F)(L)(F)(F)(L)(F) | |
| FLFFLFFLFF(L)(F)(F)(L)(F)(F)(L) | |
| FFLFFLFFLF(F)(L)(F)(F)(L)(F)(F) | |
| dLd LdLdLdL(d)(L)(d)(L)(d)(L)(d) | |
| LdLdLdLdL(d)(L)(d)(L)(d)(L)(d)(L) | |
| MLMLMLMLML(M)(L)(M)(L)(M)(L)(M) | |
| LMLMLMLML(M)(L)(M)(L)(M)(L)(M)(L) | |
| FLFLFLFLFL(F)(L)(F)(L)(F)(L)(F) | |
| LFLFLFLFF(F)(L)(F)(L)(F)(L)(F)(L) | |

Wherein L=LNA unit, d=DNA units, M=2′MOE RNA, F=2′Fluoro and ‘x’=as defined herein. It will be recognized that for longer oligomers the above patterns may be repeated, and for shorter, a corresponding fraction of the above motifs may be used—beginning from the 5′ end, or from the 3′ end and residues in brackets are optional

In one embodiment, the invention further provides for a oligomer wherein said oligomer (or contiguous nucleobase sequence) comprises either at least one phosphorothioate linkage and/or at least one 3′ terminal LNA unit, and/or at least one 5′ terminal LNA unit.

### Proteins

The invention further provides for an isolated, or purified, soluble form of TNFalpha receptor comprises a deletion in the trans-membrane binding domain encoded by exon 7, wherein said TNFalpha receptor is selected from the TNFalpha receptor TNFRSF1A or TNFRSF1B, or a variant, fragment or homologue thereof.

In one embodiment, the isolated, or purified, soluble form of TNFalpha receptor according to the invention lacks the trans-membrane binding domain encoded by exon 7.

In one embodiment, the isolated, or purified, soluble form of TNFalpha receptor is the human TNFR1 TNFalpha receptor (residues 1-455, or residues 30-455 of SEQ ID NO 123, or a variant, fragment or homologue thereof), wherein said deletion is between residues 209 and 246 (or region which corresponds to residues 209 and 246 of SEQ ID No 123).

In one embodiment, the isolated, or purified, soluble form of TNFalpha has a sequence consisting of residues 1-208 or residues 30-208 of SEQ ID NO 119, or is a variant, fragment or homologue thereof.

In one embodiment, the isolated, or purified, soluble form of TNFalpha receptor is the human TNFR2 TNFalpha receptor (residues 1-435, or residues 23435 of SEQ ID NO 127, or a variant, fragment or homologue thereof, wherein said deletion is between residues 263 and 289 (or region which corresponds to residues 209 and 246 of SEQ ID No 123).

In one embodiment, the isolated, or purified, soluble form of TNFalpha receptor has a sequence consisting of residues 1-262 or 23-262 of SEQ ID NO 127, or is a variant, fragment or homologue thereof.

In one preferred embodiment, the soluble form of the TNFalpha receptor is both isolated and purified.

One embodiment of the present invention is a protein, either full length or mature, which is encoded by a cDNA derived from a mammalian TNFR gene, and in the cDNA exon 6 is followed directly by exon 8 and as a result lacks exon 7. Furthermore the protein can bind TNF, preferably TNF-α, and can act as a TNF, preferably TNF-α, antagonist. Preferably, TNFR of the present invention is capable of inhibition of TNF-mediated cytotoxicity to a greater extent than the soluble extracellular domain alone, and more preferably, to an extent comparable to or greater than TNFR:Fc. Mammalian TNFR according to the present disclosure includes, but is not limited to, human, primate, murine, canine, feline, bovine, ovine, equine, and porcine TNFR. Furthermore, mammalian TNFR according to the present disclosure includes, but is not limited to, a protein sequence that results from one or more single nucleotide polymorphisms, such as for example those disclosed in EP Pat. Appl. 1,172,444, as long as the protein retains a comparable biological activity to the reference sequence with which it is being compared.

In one embodiment, the mammalian TNFR is a mammalian TNFR1, preferably a human TNFR1. For human TNFR1 two non-limiting examples of this embodiment are given by huTNFR1 Δ7 which includes the signal sequence as shown in SEQ ID No: 122 and mature huTNFR1 Δ7 (amino acids 30-417 of SEQ ID No: 122) which lacks the signal sequence. The sequences of these TNFR1 Δ7 proteins are either amino acids 1-208 of wild type human TNFR1 (SEQ ID No: 118) which includes the signal sequence or 30-208 of wild type human TNFR1 for mature huTNFR1 Δ7 which lacks the signal sequence, and in either case is followed immediately by amino acids 247-455 of wild type human TNFR1.

In another preferred embodiment, the mammalian TNFR is a mammalian TNFR2, most preferably a human TNFR2. For human TNFR2 two non-limiting examples of this embodiment are given by huTNFR2 Δ7 which includes the signal sequence as shown in SEQ ID No: 126 or mature huTNFR2 Δ7 (amino acids 23-435 of SEQ ID No: 126) which lacks the signal sequence. The sequences of these huTNFR2 Δ7 proteins are either amino acids 1-262 of wild type human TNFR2 (SEQ ID No: 120) which includes the signal sequence or 23-262 of wild type human TNFR2 for mature huTNFR2 Δ7 which lacks the signal sequence, followed in either case by the amino acid glutamate, because of the creation of a unique codon at the exon 6-8 junction, which is followed by amino acids 290-461 of wild type human TNFR2.

The proteins of the present invention also include those proteins that are chemically modified. Chemical modification of a protein refers to a protein where at least one of its amino acid residues is modified by either natural processes, such as processing or other post-translational modifications, or by chemical modification techniques known in the art. Such modifications include, but are not limited to, acetylation, acylation, amidation, ADP-ribosylation, glycosylation, methylation, pegylation, prenylation, phosphorylation, or cholesterol conjugation.

The proteins of the present invention may, in one embodiment, also include variants, fragments and homologues of the proteins of the invention. However, such proteins comprise a deletion in the amino acid sequence which is encoded by exon 7 or exon 8, as explained herein.

### Nucleic Acids

The invention further provides a nucleic acid encoding the soluble form of TNFalpha receptor according to the invention.

In one embodiment, the nucleic acid is selected from the group consisting of: nucleotides 1-1251 of SEQ ID NO 121, 88-1251 of SEQ ID NO 121, 1-1305 of SEQ ID NO 125 and 67-1305 of SEQ ID NO 125, or variant, homologue or fragment thereof, including a nucleic acid which encodes the same primary amino acid sequence as the nucleic acid, i.e. due to the degeneracy of the genetic code.

One embodiment of the present invention is a nucleic acid that encodes a protein, either full length or mature, which is encoded by a cDNA derived from a mammalian TNFR gene, and in the cDNA exon 6 is followed directly by exon 8 and as a result lacks exon 7.

Such sequences are preferably provided in the form of an open reading frame uninterrupted by internal nontranslated sequences, or introns, which are typically present in eukaryotic genes. Genomic DNA containing the relevant sequences can also be used. In one embodiment, the nucleic acid is either an mRNA or a cDNA. In another embodiment, it is genomic DNA.

In one embodiment, the mammalian TNFR is a mammalian TNFR1. For this embodiment, the mammalian TNFR1 is preferably a human TNFR1. For human TNFR1, two non-limiting examples of this embodiment are nucleic acids which encode the huTNFR1 Δ7 which includes the signal sequence as shown in SEQ ID No: 122 and mature huTNFR1 Δ7 (amino acids 30-417 of SEQ ID No: 122) which lacks the signal sequence. Preferably, the sequences of these huTNFR1 Δ7 nucleic acids are nucleotides 1-1251 of SEQ ID No: 121, which includes the signal sequence and nucleotides 88-1251 of SEQ ID No: 121 which lacks the signal sequence. The sequences of these huTNFR1 Δ7 nucleic acids are either nucleotides 1-625 of wild type human TNFR1 (SEQ ID No: 117) which includes the signal sequence or 88-625 of wild type human TNFR1 for mature huTNFR2 Δ7 which lacks the signal sequence, and in either case is followed immediately by amino acids 740-1368 of wild type human TNFR1.

In another preferred embodiment, the mammalian TNFR is a mammalian TNFR2, most preferably a human TNFR2. For human TNFR2, two non-limiting examples of this embodiment are nucleic acids which encode the huTNFR2 Δ7 which includes the signal sequence as shown in SEQ ID No: 126 or mature huTNFR2 Δ7 (amino acids 23-435 of SEQ ID No: 126) which lacks the signal sequence. Preferably, the sequences of these huTNFR2 Δ7 nucleic acids are nucleotides 1-1305 of SEQ ID No: 115 which includes the signal sequence and nucleotides 67-1305 of SEQ ID No: 115 which lacks the signal sequence. The sequences of these huTNFR2 Δ7 nucleic acids are either nucleotides 1-787 of wild type human TNFR2 (SEQ ID No: 119) which includes the signal sequence or 67-787 of wild type human TNFR2 for mature huTNFR2 Δ7 which lacks the signal sequence, and in either case is followed immediately by amino acids 866-1386 of wild type human TNFR2.

The bases of the nucleic acids of the present invention can be the conventional bases cytosine, guanine, adenine and uracil or thymidine. Alternatively, modified bases can be used. Other suitable bases include, but are not limited to, 5-methylcytosine (^{Me}C), isocytosine, pseudoisocytosine, 5-bromouracil, 5-propynyluracil, 5-propyny-6,5-methylthiazoleuracil, 6-aminopurine, 2-aminopurine, inosine, 2,6-diaminopurine, 7-propyne-7-deazaadenine, 7-propyne-7-deazaguanine, 2-chloro-6-aminopurine and 9-(aminoethoxy)phenoxazine.

Suitable nucleic acids of the present invention include numerous alternative chemistries. For example, suitable nucleic acids of the present invention include, but are not limited to, those wherein at least one of the internucleotide bridging phosphate residues is a modified phosphate, such as phosphorothioate, methyl phosphonate, methyl phosphonothioate, phosphoromorpholidate, phosphoropiperazidate, and phosphoroamidate. In another non-limiting example, suitable nucleic acids of the present invention include those wherein at least one of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl).

Nucleic acids of the present invention also include, but are not limited to, those wherein at least one, of the nucleotides is a nucleic acid analogue. Examples of such analogues include, but are not limited to, hexitol (HNA) nucleotides, 2′O-4′C-linked bicyclic ribofuranosyl (LNA) nucleotides, peptide nucleic acid (PNA) analogues, N3′→P5′ phosphoramidate analogues, phosphorodiamidate morpholino nucleotide analogues, and combinations thereof.

Nucleic acids of the present invention include, but are not limited to, modifications of the nucleic acids involving chemically linking to the nucleic acids one or more moieties or conjugates. Such moieties include, but are not limited to, lipid moieties such as a cholesterol moiety, cholic acid, a thioether, e.g. hexyl-5-tritylthiol, a thiocholesterol, an aliphatic chain, e.g., dodecandiol or undecyl residues, a phospholipids, e.g., di-hexadecyl-rac-glycerol or triethylammonium 1,2-di-O-hexadecyl-rac-glycero-3—H-phosphonate, a polyamine or a polyethylene glycol chain, an adamantane acetic acid, a palmityl moiety, an octadecylamine or hexylamino-carbonyl-oxycholesterol moiety.

### Expression Vectors and Host Cells

The invention also provides for a vector comprising the nucleic acid of the invention.

In one embodiment, the vector comprises an expression cassette capable of driving the expression of said nucleic acid in a host cell.

The invention also provides for a host cell comprising the nucleic acid or the vector according to the invention.

The invention also provides for a method for the preparation of a soluble form of TNFalpha receptor, said method comprising the step of culturing the host cell according to the invention under conditions which allow the expression of said nucleic acid, and subsequently isolating said soluble form of TNFalpha receptor from said host cells.

The present invention provides expression vectors to amplify or express DNA encoding mammalian TNFR of the current invention. The present invention also provides host cells transformed with the foregoing expression vectors. Expression vectors are replicable DNA constructs which have synthetic or cDNA-derived DNA fragments encoding mammalian TNFR or bioequivalent analogues operably linked to suitable transcriptional or translational regulatory elements derived from mammalian, microbial, viral, or insect genes. A transcriptional unit generally comprises an assembly of (a) a genetic element or elements having a regulatory role in gene expression, such as, transcriptional promoters or enhancers, (b) a structural or coding sequence which is transcribed into mRNA and translated into protein, and (c) appropriate transcription and translation initiation and termination sequences. Such regulatory elements can include an operator sequence to control transcription, and a sequence encoding suitable mRNA ribosomal binding sites. The ability to replicate in a host, usually conferred by an origin of replication, and a selection gene to facilitate recognition of transformants, can additionally be incorporated.

DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Generally, operably linked means contiguous and, in the case of secretory leaders, contiguous and in reading frame. Structural elements intended for use in yeast expression systems preferably include a leader sequence enabling extracellular secretion of translated protein by a host cell. Alternatively, where recombinant protein is expressed without a leader or transport sequence, it may include an N-terminal methionine residue. This residue may optionally be subsequently cleaved from the expressed protein to provide a final product.

Mammalian TNFR DNA is expressed or amplified in a recombinant expression system comprising a substantially homogeneous monoculture of suitable host microorganisms, for example, bacteria such as E. coli or yeast such as S. cerevisiae, which have stably integrated (by transformation or transfection) a recombinant transcriptional unit into chromosomal DNA or carry the recombinant transcriptional unit as a component of a resident plasmid. Recombinant expression systems as defined herein will express heterologous protein either constitutively or upon induction of the regulatory elements linked to the DNA sequence or synthetic gene to be expressed.

Transformed host cells are cells which have been transformed or transfected with mammalian TNFR vectors constructed using recombinant DNA techniques. Transformed host cells ordinarily express TNFR, but host cells transformed for purposes of cloning or amplifying TNFR DNA do not need to express TNFR. Suitable host cells for expression of mammalian TNFR include prokaryotes, yeast, fungi, or higher eukaryotic cells. Prokaryotes include grain negative or gram positive organisms, for example E. coli or bacilli. Higher eukaryotic cells include, but are not limited to, established insect and mammalian cell lines. Cell-free translation systems can also be employed to produce mammalian TNFR using RNAs derived from the DNA constructs of the present invention. Appropriate cloning and expression vectors for use with bacterial, fungal, yeast, and mammalian cellular hosts are well known in the art.

Prokaryotic expression hosts may be used for expression of TNFR that do not require extensive proteolytic and disulfide processing. Prokaryotic expression vectors generally comprise one or more phenotypic selectable markers, for example a gene encoding proteins conferring antibiotic resistance or supplying an autotrophic requirement, and an origin of replication recognized by the host to ensure amplification within the host. Suitable prokaryotic hosts for transformation include E. coli, Bacillus subtilis, Salmonella typhimurium, and various species within the genera Pseudomonas, Streptomyces, and Staphylococcus, although others can also be employed as a matter of choice.

Useful expression vectors for bacterial use can comprise a selectable marker and bacterial origin of replication derived from commercially available plasmids comprising genetic elements of the well known cloning vector pBR322 (ATCC 37017). These pBR322 “backbone” sections are combined with an appropriate promoter and the structural sequence to be expressed. pBR322 contains genes for ampicillin and tetracycline resistance and thus provides simple means for identifying transformed cells. Such commercial vectors include, for example, the series of Novagen® pET vectors (EMD Biosciences, Inc., Madison, Wis.).

Promoters commonly used in recombinant microbial expression vectors include the lactose promoter system, and the λ P_{L} promoter, the T7 promoter, and the T7 lac promoter. A particularly useful bacterial expression system, Novagen® pET system (EMD Biosciences, Inc., Madison, Wis.) employs a T7 or T7 lac promoter and E. coli strain, such as BL21 (DE3) which contain a chromosomal copy of the T7 RNA polymerase gene.

TNFR proteins can also be expressed in yeast and fungal hosts, preferably from the genus Saccharomyces, such as S. cerevisiae. Yeast of other genera, such as Pichia or Kluyveromyces can also be employed. Yeast vectors will generally contain an origin of replication from the 2μ yeast plasmid or an autonomously replicating sequence (ARS), promoter, DNA encoding TNFR, sequences for polyadenylation and transcription termination and a selection gene. Preferably, yeast vectors will include an origin of replication and selectable marker permitting transformation of both yeast and E. coli, e.g., the ampicillin resistance gene of E. coli and S. cerevisiae TRP1 or URA3 gene, which provides a selection marker for a mutant strain of yeast lacking the ability to grow in tryptophan or uracil, respectively, and a promoter derived from a highly expressed yeast gene to induce transcription of a structural sequence downstream. The presence of the TRP1 or URA3 lesion in the yeast host cell genome then provides an effective environment for detecting transformation by growth in the absence of tryptophan or uracil, respectively.

Suitable promoter sequences in yeast vectors include the promoters for metallothionein, 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are well known in the art.

Preferred yeast vectors can be assembled using DNA sequences from pUC18 for selection and replication in E. coli (Amp^{r} gene and origin of replication) and yeast DNA sequences including a glucose-repressible ADH2 promoter and α-factor secretion leader. The yeast α-factor leader, which directs secretion of heterologous proteins, can be inserted between the promoter and the structural gene to be expressed. The leader sequence can be modified to contain, near its 3′ end, one or more useful restriction sites to facilitate fusion of the leader sequence to foreign genes. Suitable yeast transformation protocols are known to those of skill in the art.

Host strains transformed by vectors comprising the ADH2 promoter may be grown for expression in a rich medium consisting of 1% yeast extract, 2% peptone, and 1% or 4% glucose supplemented with 80 mg/ml adenine and 80 mg/ml uracil. Derepression of the ADH2 promoter occurs upon exhaustion of medium glucose. Crude yeast supernatants are harvested by filtration and held at 4° C. prior to further purification.

Various mammalian or insect cell culture systems are also advantageously employed to express TNFR protein. Expression of recombinant proteins in mammalian cells is particularly preferred because such proteins are generally correctly folded, appropriately modified and completely functional. Examples of suitable mammalian host cell lines include the COS-7 lines of monkey kidney cells, and other cell lines capable of expressing an appropriate vector including, for example, L cells, such as L929, C127, 3T3, Chinese hamster ovary (CHO), HeLa and BHK cell lines. Mammalian expression vectors can comprise nontranscribed elements such as an origin of replication, a suitable promoter, for example, the CMVie promoter, the chicken beta-actin promoter, or the composite hEF1-HTLV promoter, and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are known to those of skill in the art.

The transcriptional and translational control sequences in expression vectors to be used in transforming vertebrate cells can be provided by viral sources. For example, commonly used promoters and enhancers are derived from Polyoma, Adenovirus 2, Simian Virus 40 (SV40), human cytomegalovirus, such as the CMVie promoter, HTLV, such as the composite hEF1-HTLV promoter. DNA sequences derived from the SV40 viral genome, for example, SV40 origin, early and late promoter, enhancer, splice, and polyadenylation sites can be used to provide the other genetic elements required for expression of a heterologous DNA sequence.

Further, mammalian genomic TNFR promoter, such as control and/or signal sequences can be utilized, provided such control sequences are compatible with the host cell chosen.

In preferred aspects of the present invention, recombinant expression vectors comprising TNFR cDNAs are stably integrated into a host cell's DNA.

### Protein Expression and Purification:

When mammalian or insect cells are used, properly expressed TNFR protein will be secreted into the extracellular media. The protein is recovered from the media, and is concentrated and is purified using standard biochemical techniques. After expression in mammalian cells by lentiviral or AAV transduction, plasmid transfection, or any similar procedure, or in insect cells after baculoviral transduction, the extracellular media of these cells is concentrated using concentration filters with an appropriate molecular weight cutoff, such as Amicon® filtration units. To avoid loss of TNFR protein, the filter should allow proteins to flow through that are at or below 50 kDal.

When TNFR protein is expressed in bacterial culture it can be purified by standard biochemical techniques. Bacteria are lysed, and the cellular extract containing the TNFR is desalted and is concentrated.

In either case, the TNFR protein is preferably purified by affinity chromatography. The use of column chromatography with an affinity matrix comprising TNF-α is preferred. Alternatively, an affinity purification tag can be added to either the N- or the C-terminus of the TNFR protein. For example, a polyhistidine-tag (His-tag), which is an amino acid motif with at least six histidines, can be used for this purpose (Hengen, P., 1995, Trends Biochem. Sci. 20:285-86). The addition of a His-tag can be achieved by the in-frame addition of a nucleotide sequence encoding the His-tag directly to either the 5′ or 3′ end of the TNFR open reading frame in an expression vector. One such nucleotide sequence for the addition of a C-terminal His-tag is given in SEQ ID No: 126. When a His-tag is incorporated into the protein, a nickel or cobalt affinity column is employed to purify the tagged TNFR, and the His-tag can optionally then be cleaved. Other suitable affinity purification tags and methods of purification of proteins with those tags are well known in the art.

Alternatively, a non-affinity based purification scheme can be used, involving fractionation of the TNFR extracts on a series of columns that separate the protein based on size (size exclusion chromatography), charge (anion and cation exchange chromatography) and hydrophobicity (reverse phase chromatography). High performance liquid chromatography can be used to facilitate these steps.

Other methods for the expression and purification of TNFR proteins are well known (See, e.g., U.S. Pat. No. 5,605,690 to Jacobs).

### DEFINITIONS

The term “internucleoside linkage group” is intended to mean a group capable of covalently coupling together two nucleobases, such as between DNA units, between DNA units and nucleotide analogues, between two non-LNA units, between a non-LNA unit and an LNA unit, and between two LNA units, etc. Preferred examples include phosphate, phosphodiester groups and phosphorothioate groups.

Herein, the term “nitrogenous base” is intended to cover purines and pyrimidines, such as the DNA nucleobases A, C, T and G, the RNA nucleobases A, C, U and G, as well as non-DNA/RNA nucleobases, such as 5-methylcytosine (^{Me}C), isocytosine, pseudoisocytosine, 5-bromouracil, 5-propynyluracil, 5-propyny-6-fluorouracil, 5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine, in particular ^{Me}C. It will be understood that the actual selection of the non-DNA/RNA nucleobase will depend on the corresponding (or matching) nucleotide present in the microRNA strand which the oligonucleotide is intended to target. For example, in case the corresponding nucleotide is G it will normally be necessary to select a non-DNA/RNA nucleobase which is capable of establishing hydrogen bonds to G. In this specific case, where the corresponding nucleotide is G, a typical example of a preferred non-DNA/RNA nucleobase is ^{Me}C.

As used herein, the terms “tumor necrosis factor receptor”, “TNF receptor”, and “TNFR” refer to proteins having amino acid sequences of or which are substantially similar to native mammalian TNF receptor sequences, and which are capable of binding TNF molecules. In this context, a “native” receptor or gene for such a receptor, means a receptor or gene that occurs in nature, as well as the naturally-occurring allelic variations of such receptors and genes.

The term “mature” as used in connection with a TNFR means a protein expressed in a form lacking a leader or signal sequence as may be present in full-length transcripts of a native gene.

The nomenclature for TNFR proteins as used herein follows the convention of naming the protein (e.g., TNFR2) preceded by a species designation, e.g., hu (for human) or mu (for murine), followed by a Δ (to designate a deletion) and the number of the exon(s) deleted. For example, huTNFR2 Δ7 refers to human TNFR2 lacking exon 7. In the absence of any species designation, TNFR refers generically to mammalian TNFR.

The term “secreted” means that the protein is soluble, i.e., that it is not bound to the cell membrane. In this context, a form will be soluble if using conventional assays known to one of skill in the art most of this form can be detected in fractions that are not associated with the membrane, e.g., in cellular supernatants or serum.

The term “stable” means that the secreted TNFR form is detectable using conventional assays by one of skill in the art, such as, western blots, ELISA assays in harvested cells, cellular supernatants, or serum.

As used herein, the terms “tumor necrosis factor” and “TNF” refer to the naturally-occurring protein ligands that bind to TNF receptors. TNF includes, but is not limited to, TNF-α and TNF-β.

As used herein, the term “an inflammatory disease or condition” refers to a disease, disorder, or other medical condition that at least in part results from or is aggravated by the binding of TNF to its receptor. Such diseases or conditions include, but are not limited to, those associated with increased levels of TNF, increased levels of TNF receptor, or increased sensitization or deregulation of the corresponding signaling pathway. The term also encompasses diseases and conditions for which known TNF antagonists have been shown useful. Examples of inflammatory diseases or conditions include, but are not limited to, rheumatoid arthritis, juvenile rheumatoid arthritis, psoriasis, psoriatic arthritis, ankylosing spondylitis, inflammatory bowel disease (including Crohn's disease and ulcerative colitis), hepatitis, sepsis, alcoholic liver disease, and non-alcoholic steatosis.

As used herein, the term “hepatitis” refers to a gastroenterological disease, condition, or disorder that is characterized, at least in part, by inflammation of the liver. Examples of hepatitis include, but are not limited to, hepatitis associated with hepatitis A virus, hepatitis B virus, hepatitis C virus, or liver inflammation associated with ischemia/reperfusion.

As used herein, the term “TNF antagonist” means that the protein is capable of measurable inhibition of TNF-mediated cytotoxicity using standard assays as are well known in the art. (See, e.g L929 cytotoxicity assay as described in the Examples below).

The term “binds TNF” means that the protein can bind detectable levels of TNF, preferably TNF-α, as measured by standard binding assays as are well known in the art (See, e.g. U.S. Pat. No. 5,945,397 to Smith, cols. 16-17). Preferably, receptors of the present invention are capable of binding greater than 0.1 nmoles TNF-α/nmole receptor, and more preferably, greater than 0.5 nmoles TNF-α/mole receptor using standard binding assays.

As used herein, the term “regulatory element” refers to a nucleotide sequence involved in an interaction of molecules that contributes to the functional regulation of a nucleic acid, including but not limited to, replication, duplication, transcription, splicing, translation, or degradation of the nucleic acid. The regulation may be enhancing or inhibitory in nature. Regulatory elements known in the art include, for example, transcriptional regulatory sequences such as promoters and enhancers. A promoter is a DNA region that is capable under certain conditions of aiding the initiation of transcription of a coding region usually located downstream (in the 3′ direction) from the promoter. An expression vector typically comprises such regulatory elements operably linked to the nucleic acid of the invention.

The terms “oligomer” and “splice switching oligomer” and “oligonucleotide” are used interchangeably herein.

As used herein, the term “operably linked” refers to a juxtaposition of genetic elements, wherein the elements are in a relationship permitting them to operate in the expected manner. For example, a promoter is operably linked to a coding region if the promoter helps initiate transcription of the coding sequence (such as in an expression vector). As long as this functional relationship is maintained, there can be intervening residues between the promoter and the coding region.

As used herein, the terms “transformation” or “transfection” refer to the insertion of an exogenous nucleic acid into a cell, irrespective of the method used for the insertion, for example, lipofection, transduction, infection or electroporation. The exogenous nucleic acid can be maintained as a non-integrated vector, for example, a plasmid, or alternatively, can be integrated into the cell's genome.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. or viral vectors (e.g., replication defective retroviruses, adenoviruses and adeno-associated viruses).

As used herein, the term “isolated protein” refers to a protein or polypeptide that is not naturally-occurring and/or is separated from one or more components that are naturally associated with it.

As used herein, the term “isolated nucleic acid” refers to a nucleic acid that is not naturally-occurring and/or is in the form of a separate fragment or as a component of a larger construct, which has been derived from a nucleic acid isolated at least once in substantially pure form, i.e., free of contaminating endogenous materials, and in a quantity or concentration enabling identification and manipulation by standard biochemical methods, for example, using a cloning vector.

As used herein the term “purified protein” refers to a protein that is present in the substantial absence of other protein. However, such purified proteins can contain other proteins added as stabilizers, carriers, excipients, or co-therapeutics. The term “purified” as used herein preferably means at least 50% such as at least 80% by dry weight, more preferably in the range of 95-99% by weight, and most preferably at least 99.8% by weight, of protein present, excluding proteins added as stabilizers, carriers, excipients, or co-therapeutics.

As used herein, the term “altering the splicing of a pre-mRNA” refers to altering the splicing of a cellular pre-mRNA target resulting in an altered ratio of splice products. Such an alteration of splicing can be detected by a variety of techniques well known to one of skill in the art. For example, RT-PCR on total cellular RNA can be used to detect the ratio of splice products in the presence and the absence of an SSO.

As used herein, the term “complementary” is used to indicate a sufficient degree of complementarity or precise pairing such that stable and specific binding occurs between an oligonucleotide and a DNA or RNA containing the target sequence. It is understood in the art that the sequence of an oligonucleotide need not be 100% complementary to that of its target. For example, for an SSO there is a sufficient degree of complementarity when, under conditions which permit splicing, binding to the target will occur and non-specific binding will be avoided. However, it is preferred that the oligonucleotide or contiguous nucleobase sequence is fully (i.e. perfectly) complementary to the target sequence (such as the region of SEQ ID NO 1-4, referred to herein).

The terms “corresponding to” and “corresponds to” as used in the context of oligonucleotides refers to the comparison between either a nucleobase sequence of the compound of the invention, and the reverse complement thereof, or in one embodiment between a nucleobase sequence and an equivalent (identical) nucleobase sequence which may for example comprise other nucleobases but retains the same base sequence, or complement thereof. Nucleotide analogues are compared directly to their equivalent or corresponding natural nucleotides. Sequences which form the reverse complement of a sequence are referred to as the complement sequence of the sequence.

When referring to the length of a nucleotide molecule as referred to herein, the length corresponds to the number of monomer units, i.e. nucleobases, irrespective as to whether those monomer units are nucleotides or nucleotide analogues. With respect to nucleobases, the terms monomer and unit are used interchangeably herein.

It should be understood that when the term “about” is used in the context of specific values or ranges of values, the disclosure should be read as to include the specific value or range referred to.

The term “variant” as used in herein in the context of a protein or polypeptide (sequence), refers to a polypeptide which is prepared from the original (parent) polypeptide, or using the sequence information from the polypeptide, by insertion, deletion or substitution of one or more amino acids in said sequence, i.e. at least one amino acids, but preferably less than 50 amino acids, such as less than 40, less than 30, less than 20, or less than 10 amino acids, such as 1 amino acid, 1-2 amino acids, 1-3 amino acids, 1-4 amino acids, 1-5 amino acids.

The term “homologue” as used herein in the context of a protein or polypeptide (sequence), refers to a polypeptide which is at least 70% homologous, such as at least 80% homologous, such as at least 85% homologous, or at least 90% homologous, such as at least 95%, 96%, 97%, 98% or 99% homologous to said polypeptide sequence. Homology between two polypeptide sequences may be determined using ClustalW alignment algorithm using the Blosum 62 algorithm, with Gap Extent=0.5, Gap open=10 (see http:/www.ebi.ac.uk/emboss/align/index.html). The alignment may, in one embodiment be a local alignment (water) or a separate embodiment be a global alignment (needle). As the homologues of the exon deletion TNFR proteins referred to herein also comprise deletion in the respective exon, a global alignment may be preferred.

The term “fragment” as used herein in the context of a protein or polypeptide (sequence), refers to a polypeptide which consists of only a part of the polypeptide sequence. A fragment may therefore comprise at least 5% such as at least 10% of said polypeptide sequence, including at least 20%, 30%, 40%, 50%, 60%, 70%, 80%, 90% or 95% of said polypeptide sequence.

The above definitions of variant, fragment and homologue also apply to nucleic acid sequences, although the homology algorithm used is DNAfull. Obviously, when referring to nucleic acid variant, fragment or homologue, the terms protein, polypeptide and amino acid should be replaced with nucleic acid, polynucleotide or nucleobase/nucleotide accordingly.

As used herein, the terms “membrane bound form” or “integral membrane form” refer to proteins having amino acid sequences that span a cell membrane, with amino acid sequences on each side of the membrane.

As used herein, the term “stable, secreted, ligand-binding form” or as it is sometimes known “stable, soluble, ligand-binding form.” (where the terms “secreted” and “soluble” are synonymous and interchangeable herein) refer to proteins that are related to the native membrane bound form receptors, in such a way that they are secreted and stable and still capable of binding to the corresponding ligand. It should be noted that these forms are not defined by whether or not such secreted forms are physiological, only that the products of such splice variants would be secreted, stable, and still capable of ligand-binding when produced.

The term “secreted” means that the form is soluble, i.e., that it is no longer bound to the cell membrane. In this context, a form will be soluble if using conventional assays known to one of skill in the art most of this form can be detected in fractions that are not associated with the membrane, e.g., in cellular supernatants or serum.

The term “stable” means that the secreted form is detectable using conventional assays by one of skill in the art. For example, western blots, ELISA assays can be used to detect the form from harvested cells, cellular supernatants, or serum from patients.

The term “ligand-binding” means that the form retains at least some significant level, although not necessarily all, of the specific ligand-binding activity of the corresponding integral membrane form.

As used herein, the term “to reduce the activity of a ligand” refers to any action that leads to a decrease in transmission of an intracellular signal resulting from the ligand binding to or interaction with the receptor. For example, activity can be reduced by binding of the ligand to a soluble form of its receptor or by decreasing the quantity of the membrane form of its receptor available to bind the ligand.

### Pharmaceutical Compositions and Preparations

Other embodiments of the invention are pharmaceutical compositions comprising the oligomers, proteins and nucleic acids according to the invention.

The oligomers, nucleic acids and proteins of the present invention may be admixed, encapsulated, conjugated, or otherwise associated with other molecules, molecule structures, or mixtures of compounds, as for example liposomes, receptor targeted molecules, oral, rectal, topical or other formulations, for assisting in uptake, distribution, and/or absorption.

Formulations of the present invention comprise the oligomers, nucleic acids or proteins according to the invention in a physiologically or pharmaceutically acceptable carrier, such as an aqueous carrier. Thus formulations for use in the present invention include, but are not limited to, those suitable for parenteral administration including intra-articular, intraperitoneal, intravenous, intraarterial, subcutaneous, or intramuscular injection or infusion, as well as those suitable for topical, ophthalmic, vaginal, oral, rectal or pulmonary administration (including inhalation or insufflation of powders or aerosols, including by nebulizer, intratracheal, and intranasal delivery). The formulations may conveniently be presented in unit dosage form and may be prepared by any of the methods well known in the art. The most suitable route of administration in any given case may depend upon the subject, the nature and severity of the condition being treated, and the particular active compound which is being used.

Pharmaceutical compositions of the present invention include, but are not limited to, physiologically and pharmaceutically acceptable salts, i.e., salts that retain the desired biological activity of the parent compound and do not impart undesired toxicological properties. Examples of such salts are (a) salts formed with cations such as sodium, potassium, NH₄⁺, magnesium, calcium, polyamines such as spermine and spermidine, etc.; (b) acid addition salts formed with inorganic acids, for example, hydrochloric acid, hydrobromic acid, sulfuric acid, phosphoric acid, nitric acid and the like; and (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, napthalenesulfonic acid, methanesulfonic acid, p-toluenesulfonic acid, napthalenedisulfonic acid, polygalacturonic acid, and the like.

The present invention provides for the use of the oligomers, proteins and nucleic acids as set forth above for the preparation of a medicament for treating a patient afflicted with an inflammatory disorder involving excessive activity of TNF, as discussed below. In the manufacture of a medicament according to the invention, the oligomers, nucleic acids and proteins of the present invention are typically admixed with, inter alia, an acceptable carrier. The carrier must, of course, be acceptable in the sense of being compatible with other ingredients in the formulation and must not be deleterious to the patient. The carrier may be a solid or liquid. Oligomers, nucleic acids and proteins of the present invention are incorporated in formulations, which may be prepared by any of the well known techniques of pharmacy consisting essentially of admixing the components, optionally including one or more accessory therapeutic ingredients.

Formulations of the present invention may comprise sterile aqueous and non-aqueous injection solutions of the active compounds, which preparations are preferably isotonic with the blood of the intended recipient and essentially pyrogen free. These preparations may contain anti-oxidants, buffers, bacteriostats, and solutes which render the formulation isotonic with the blood of the intended recipient. Aqueous and non-aqueous sterile suspensions can include, but are not limited to, suspending agents and thickening agents. The formulations may be presented in unit dose or multi-dose containers, for example, sealed ampoules and vials, and may be stored in freeze-dried (lyophilized) condition requiring only the addition of the sterile liquid carrier, for example, saline or water-for-injection immediately prior to use.

In the formulation the oligomers, nucleic acids and proteins of the present invention may be contained within a particle or vesicle, such as a liposome or microcrystal, which may be suitable for parenteral administration. The particles may be of any suitable structure, such as unilamellar or plurilameller, so long as the oligomers, nucleic acids and proteins of the present invention are contained therein. Positively charged lipids such as N-[1-(2,3-dioleoyloxy)propyl]-N,N,N-trimethyl-ammoniummethylsulfate, or “DOTAP,” are particularly preferred for such particles and vesicles. The preparation of such lipid particles is well known (See references in U.S. Pat. No. 5,976,879 col. 6).

Accordingly one embodiment of the invention is a method of treating an inflammatory disease or condition by administering a stable, secreted, ligand-binding form of a TNF receptor, thereby decreasing the activity of TNF for the receptor. In another embodiment, the invention is a method of treating an inflammatory disease or condition by administering an oligonucleotide that encodes a stable, secreted, ligand-binding form of a TNF receptor, thereby decreasing the activity of TNF for the receptor. In another embodiment, the invention is a method of producing a stable, secreted, ligand-binding form of a TNF receptor.

The following aspects of the present invention discussed below apply to the foregoing embodiments.

The methods, nucleic acids, proteins, and formulations of the present invention are also useful as in vitro or in vivo tools.

Embodiments of the invention can be used to treat any condition in which the medical practitioner intends to limit the effect of TNF or a signalling pathway activated by it. In particular, the invention can be used to treat an inflammatory disease. In one embodiment, the condition is an inflammatory systemic disease, e.g., rheumatoid arthritis or psoriatic arthritis. In another embodiment, the disease is an inflammatory liver disease. Examples of inflammatory liver diseases include, but are not limited to, hepatitis associated with the hepatitis A, B, or C viruses, alcoholic liver disease, and non-alcoholic steatosis. In yet another embodiment, the inflammatory disease is a skin condition such as psoriasis.

The uses of the present invention include, but are not limited to, treatment of diseases for which known TNF antagonists have been shown useful. Three specific TNF antagonists are currently FDA-approved. The drugs are etanercept (Enbrel®), infliximab (Remicade®) and adalimumab (Humira®). One or more of these drugs is approved for the treatment of rheumatoid arthritis, juvenile rheumatoid arthritis, psoriasis, psoriatic arthritis, ankylosing spondylitis, and inflammatory bowel disease (Crohn's disease or ulcerative colitis).

### Use of Proteins for the Treatment of Inflammatory Diseases:

Accordingly one embodiment of the invention is a method of treating an inflammatory disease or condition by administering SSOs to a patient, The SSOs that are administered alter the splicing of a pre-mRNA to produce a splice variant that encodes a stable, secreted, ligand-binding form of a receptor of the TNFR superfamily, thereby decreasing the activity of the ligand for that receptor. In another embodiment, the invention is a method of producing a stable, secreted, ligand-binding form of a receptor of the TNFR superfamily in a cell by administering SSOs to the cell.

For therapeutic use, purified TNFR proteins of the present invention are administered to a patient, preferably a human, for treating TNF-dependent inflammatory diseases, such as arthritis. In the treatment of humans, the use of huTNFRs is preferred. The TNFR proteins of the present invention can be administered by bolus injection, continuous infusion, sustained release from implants, or other suitable techniques. Typically, TNFR therapeutic proteins will be administered in the form of a composition comprising purified protein in conjunction with physiologically acceptable carriers, excipients or diluents. Such carriers will be nontoxic to recipients at the dosages and concentrations employed. Ordinarily, the preparation of such compositions entails combining the TNFR with buffers, antioxidants such as ascorbic acid, polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions, for example, sucrose, as diluents. Preservatives, such as benzyl alcohol may also be added. The amount and frequency of administration will depend of course, on such factors as the nature and the severity of the indication being treated, the desired response, the condition of the patient and so forth.

TNFR proteins of the present invention are administered systemically in therapeutically effective amounts preferably ranging from about 0.1 mg/kg/week to about 100 mg/kg/week. In preferred embodiments, TNFR is administered in amounts ranging from about 0.5 mg/kg/week to about 50 mg/kg/week. For local administration, dosages preferably range from about 0.01 mg/kg to about 1.0 mg/kg per injection.

### Use of Expression Vectors to Increase the Levels of a TNF Antagonist in a Mammal

The present invention provides a process of increasing the levels of a TNF antagonist in a mammal. The process includes the step of transforming cells of the mammal with an expression vector described herein, which drives expression of a TNFR as described herein.

The process is particularly useful in large mammals such as domestic pets, those used for food production, and primates. Exemplary large mammals are dogs, cats, horses cows, sheep, deer, and pigs. Exemplary primates are monkeys, apes, and humans.

The mammalian cells can be transformed either in vivo or ex vivo. When transformed in vivo, the expression vector are administered directly to the mammal, such as by injection. Means for transforming cells in vivo are well known in the art. When transformed ex vivo, cells are removed from the mammal, transformed ex vivo, and the transformed cells are reimplanted into the mammal.

The uses of the present invention include, but are not limited to, treatment of diseases for which known TNF antagonists have been shown useful. Three specific TNF antagonists are currently FDA-approved. The drugs are etanercept (Enbrel®), infliximab (Remicade®) and adalimumab (Humira®). One or more of these drugs is approved for the treatment of rheumatoid arthritis, juvenile rheumatoid arthritis, psoriasis, psoriatic arthritis, ankylosing spondylitis, and inflammatory bowel disease (Crohn's disease or ulcerative colitis).

The administration of the SSO to subjects can be accomplished using procedures developed for ASON. ASON have been successfully administered to experimental animals and human subjects by intravenous administration in saline in doses as high as 6 mg/kg three times a week (Yacysyhn, B. R., et al., 2002, Gut 51:30 (anti-ICAM-1 ASON for treatment of Crohn's disease); Stevenson, J., et al., 1999, J. Clinical Oncology 17:2227 (anti-RAF-1 ASON targeted to PBMC)). The pharmacokinetics of 2′O-MOE phosphorothioate ASON, directed towards TNF-α has been reported (Geary, R. S., et al., 2003, Drug Metabolism and Disposition 31:1419). The systemic efficacy of mixed LNA/DNA molecules has also been reported (Fluiter, K., et al., 2003, Nucleic Acids Res. 31:953).

The systemic activity of SSO in a mouse model system was investigated using 2′O-MOE phosphorothioates and PNA chemistries. Significant activity was observed in all tissues investigated except brain, stomach and dermis (Sazani, P., et al., 2002, Nature Biotechnology 20, 1228).

In general any method of administration that is useful in conventional antisense treatments can be used to administer the SSO of the invention. For testing of the SSO in cultured cells, any of the techniques that have been developed to test ASON or SSO may be used.

Formulations of the present invention comprise SSOs in a physiologically or pharmaceutically acceptable carrier, such as an aqueous carrier. Thus formulations for use in the present invention include, but are not limited to, those suitable for parenteral administration including intraperitoneal, intraarticular, intravenous, intraarterial, subcutaneous, or intramuscular injection or infusion, as well as those suitable for topical, ophthalmic, vaginal, oral, rectal or pulmonary (including inhalation or insulation of powders or aerosols, including by nebulizer, intratracheal, intranasal delivery) administration. The formulations may conveniently be presented in unit dosage form and may be prepared by any of the methods well known in the art. The most suitable route of administration in any given case may depend upon the subject, the nature and severity of the condition being treated, and the particular active compound which is being used.

Pharmaceutical compositions of the present invention include, but are not limited to, physiologically and pharmaceutically acceptable salts, i.e, salts that retain the desired biological activity of the parent compound and do not impart undesired toxicological properties. Examples of such salts are (a) salts formed with cations such as sodium, potassium, NH₄⁺, magnesium, calcium, polyamines such as spermine and spermidine, etc.; (b) acid addition salts formed with inorganic acids, for example, hydrochloric acid, hydrobromic acid, sulfuric acid, phosphoric acid, nitric acid and the like; and (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, napthalenesulfonic acid, methanesulfonic acid, p-toluenesulfonic acid, napthalenedisulfonic acid, polygalacturonic acid, and the like.

The present invention provides for the use of SSOs having the characteristics set forth above for the preparation of a medicament for increasing the ratio of a mammalian TNFR2 protein that lacks exon 7 to its corresponding membrane bound form, in a patient afflicted with an inflammatory disorder involving TNF-α, as discussed above. In the manufacture of a medicament according to the invention, the SSOs are typically admixed with, inter alia, an acceptable carrier. The carrier must, of course, be acceptable in the sense of being compatible with any other ingredients in the formulation and must not be deleterious to the patient. The carrier may be a solid or liquid. SSOs are incorporated in the formulations of the invention, which may be prepared by any of the well known techniques of pharmacy consisting essentially of admixing the components, optionally including one or more accessory therapeutic ingredients.

Formulations of the present invention may comprise sterile aqueous and non-aqueous injection solutions of the active compounds, which preparations are preferably isotonic with the blood of the intended recipient and essentially pyrogen free. These preparations may contain anti-oxidants, buffers, bacteriostats, and solutes which render the formulation isotonic with the blood of the intended recipient. Aqueous and non-aqueous sterile suspensions can include, but are not limited to, suspending agents and thickening agents. The formulations may be presented in unit dose or multi-dose containers, for example, sealed ampoules and vials, and may be stored in freeze-dried (lyophilized) condition requiring only the addition of the sterile liquid carrier, for example, saline or water-for-injection immediately prior to use.

In the formulation the SSOs may be contained within a particle or vesicle, such as a liposome, or microcrystal, which may be suitable for parenteral administration. The particles may be of any suitable structure, such as unilamellar or plurilameller, so long as the SSOs are contained therein. Positively charged lipids such as N-[1-(2,3-diolcoyloxy)propyl]-N,N,N-trimethyl-ammoniummethylsulfate, or “DOTAP,” are particularly preferred for such particles and vesicles. The preparation of such lipid particles is well known. [See references in U.S. Pat. No. 5,976,879 col. 6]

The SSO can be targeted to any element or combination of elements that regulate splicing, including the 3′splice site, the 5′ splice site, the branch point, the polypyrimidine tract, exonic splicing enhancers, exonic splicing silencers, intronic splicing enhancers, and intronic splicing silencers.

Those skilled in the art can appreciate that the invention as directed toward human TNFR2 can be practiced using SSO having a sequence that is complementary to at least 8, to at least 9, to at least 10, to at least 11, to at least 12, to at least 13, to at least 14, to at least 15, preferably between 10 and 16 nucleotides of the portions of the TNFR1 or TNFR2 gene comprising exons 7 and its adjacent introns.

SEQ ID No: 3 contains the sequence of exon 7 of TNFR2 and 50 adjacent nucleotides of the flanking introns. For example, SSO targeted to human TNFR2 can have a nucleobase sequence selected from the sequences listed in Table 4. When affinity-enhancing modifications are used, including but not limited to LNA or G-clamp nucleotides, the skilled person recognizes the length of the SSO can be correspondingly reduced.

Those skilled in the art will also recognize that the selection of SSO sequences must be made with care to avoid a self-complementary SSO, which may lead to the formation of partial “hairpin” duplex structures. In addition, high GC content should be avoided to minimize the possibility of non-specific base pairing. Furthermore, SSOs matching off-target genes, as revealed for example by BLAST, should also be avoided.

In some situations, it may be preferred to select an SSO sequence that can target a human and at least one other species. These SSOs can be used to test and to optimize the invention in said other species before being used in humans, thereby being useful for regulatory approval and drug development purposes. For example, SSOs with sequences selected from SEQ ID Nos: 14, 30, 46, 70 and 71 which target human TNFR2 are also 100% complementary to the corresponding Macaca Mullata sequences. As a result these sequences can be used to test treatments in monkeys, before being used in humans.

The following aspects of the present invention discussed below apply to the foregoing embodiments.

The length of the SSO is similar to an antisense oligonucleotide (ASON), typically between about 10 and 24 nucleotides. The invention can be practiced with SSOs of several chemistries that hybridize to RNA, but that do not activate the destruction of the RNA by RNase H, as do conventional antisense 2^{r}-deoxy oligonucleotides. The invention can be practiced using 2′O modified nucleic acid oligomers, such as 2′O-methyl or 2′O-methyloxyethyl phosphorothioate. The nucleobases do not need to be linked to sugars; so-called peptide nucleic acid oligomers or morpholine-based oligomers can be used. A comparison of these different linking chemistries is found in Sazani, P. et al, 2001, Nucleic Acids Res. 29:3695. The term splice-switching oligonucleotide is intended to cover the above forms. Those skilled in the art will appreciate the relationship between antisense oligonucleotide gapmers and SSOs. Gapmers are ASON that contain an RNase H activating region (typically a 2′-deoxyribonucleoside phosphorothioate) which is flanked by non-activating nuclease resistant oligomers. In general, any chemistry suitable for the flanking sequences in a gapmer ASON can be used in an SSO.

The SSOs of this invention may be made through the well-known technique of solid phase synthesis. Any other means for such synthesis known in the art may additionally or alternatively be used. It is well known to use similar techniques to prepare oligonucleotides such as the phosphorothioates and alkylated derivatives.

A particularly preferred chemistry is provided by locked nucleic acids (LNA) (Koshkin, A. A., et al., 1998, Tetrahedron 54:3607; Obika, S., et al., 1998, Tetrahedron Lett. 39:5401). LNA are conventional phosphodiester-linked ribonucleotides, except the ribofuranosyl moiety is made bicyclic by a bridge between the 2′O and the 4′C. This bridge constrains the conformation of ribofuranosyl ring into the conformation, the V-endo conformation, which is adopted when a oligonucleotide hybridizes to a complementary RNA. Recent advances in the synthesis of LNA are described in WO 03/095467. The bridge is most typically a methylene or an ethylene. The synthesis of 2′O,4′C-ethylene-bridged nucleic acids (ENA), as well as other LNA, is described in Morita, et al., 2003, Bioorg. and Med. Chem. 11:2211. However, alternative chemistries can be used and the 2′O may be replaced by a 2N. LNA and conventional nucleotides can be mixed to form a chimeric SSO. For example, chimeric SSO of alternating LNA and 2′deoxynucleotides or alternating LNA and 2′O-Me or 2′O-MOE can be employed. An alternative to any of these chemistries, not merely the 2′-deoxynucleotides, is a phosphorothioatediester linkage replacing phosphodiester. For in vivo use, phosphorothioate linkages are preferred.

When LNA nucleotides are employed in an SSO it is preferred that non-LNA nucleotides also be present. LNA nucleotides have such high affinities of hybridization that there can be significant non-specific binding, which may reduce the effective concentration of the free-SSO. When LNA nucleotides are used they may be alternated conveniently with 2′-deoxynucleotides. Alternating nucleotides, alternating dinucleotides or mixed patterns, e.g., LDLDLD or LLDLLD or LDDLDD can be used, When 2′-deoxynucleotides or 2′-deoxynucleoside phosphorothioates are mixed with LNA nucleotides it is important to avoid RNase H activation. It is expected that between about one third and two thirds of the LNA nucleotides of an SSO will be suitable. For example if the SSO is a 12-mer, then at least four LNA nucleotides and four conventional nucleotides will be present.

The bases of the SSO may be the conventional cytosine, guanine, adenine and uracil or thymidine. Alternatively modified bases can also be used. Of particular interest are modified bases that increase binding affinity. One non-limiting example of preferred modified bases are the so-called G-clamp or 9-(aminoethoxy)phenoxazine nucleotides, cytosine analogs that form 4 hydrogen bonds with guanosine. (Flanagan, W. M., et al., 1999, Proc. Natl. Acad. Sci. 96:3513; Holmes, S. C., 2003, Nucleic Acids Res. 31:2759).

Numerous alternative chemistries which do not activate RNase H are available. For example, suitable SSOs may be oligonucleotides wherein at least one, or all, of the internucleotide bridging phosphate residues are modified phosphates, such as methyl phosphonates, methyl phosphonothioates, phosphoromorpholidates, phosphoropiperazidates, and phosphoroamidates. For example, every other one of the internucleotide bridging phosphate residues may be modified as described. In another non-limiting example, such SSO are oligonucleotides wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described. [See references in U.S. Pat. No. 5,976,879 col. 4].

The length of the SSO (i.e. the number of monomers in the oligomer) will be from about 10 to about 30 bases in length. In one embodiment, 20 bases of 2′O-Me-ribonucleosides phosphorothioates are effective. Those skilled in the art appreciate that when affinity-increasing chemical modifications are used, the SSO can be shorter and still retain specificity. Those skilled in the art will further appreciate that an upper limit on the size of the SSO is imposed by the need to maintain specific recognition of the target sequence, and to avoid secondary-structure forming self hybridization of the SSO and by the limitations of gaining cell entry. These limitations imply that an SSO of increasing length (above and beyond a certain length which will depend on the affinity of the SSO) will be more frequently found to be less specific, inactive or poorly active.

SSOs of the invention include, but are not limited to, modifications of the SSO involving chemically linking to the SSO one or more moieties or conjugates which enhance the activity, cellular distribution or cellular uptake of the SSO. Such moieties include, but are not limited to, lipid moieties such as a cholesterol moiety, cholic acid, a thioether, e.g. hexyl-S-tritylthiol, a thiocholesterol, an aliphatic chain, e.g., dodecandiol or undecyl residues, a phospholipids, e.g., di-hexadecyl-rac-glycerol or triethylammonium 1,2-di-O-hexadecyl-rac-glycero-3-H-phosphonate, a polyamine or a polyethylene glycol chain, an adamantane acetic acid, a palmityl moiety, an octadecylamine or hexylamino-carbonyl-oxycholesterol moiety.

It is not necessary for all positions in a given SSO to be uniformly modified, and in fact more than one of the aforementioned modifications may be incorporated in a single compound or even at a single nucleoside within an SSO.

The SSOs may be admixed, encapsulated, conjugated, or otherwise associated with other molecules, molecule structures, or mixtures of compounds, as for example liposomes, receptor targeted molecules, oral, rectal, topical or other formulation, for assisting in uptake, distribution, and/or absorption.

Those skilled in the art appreciate that cellular differentiation includes, but is not limited to, differentiation of the spliceosome. Accordingly, the activity of any particular SSO of the invention can depend upon the cell type into which they are introduced. For example, SSOs which are effective in cell type may be ineffective in another cell type.

The methods, oligonucleotides, and formulations of the present invention are also useful as in vitro or in vivo tools to examine splicing in human or animal genes. Such methods can be carried out by the procedures described herein, or modifications thereof which will be apparent to skilled persons.

The invention can be used to treat any condition in which the medical practitioner intends to limit the effect of a TNF superfamily ligand or the signalling pathway activated by such ligand. In particular, the invention can be used to treat an inflammatory disease. In one embodiment, the condition is an inflammatory systemic disease, e.g., rheumatoid arthritis or psoriatic arthritis. In mother embodiment, the disease is an inflammatory liver disease. Examples of inflammatory liver diseases include, but are not limited to, hepatitis associated with the hepatitis A, B, or C viruses, alcoholic liver disease, and non-alcoholic steatosis. In yet another embodiment, the inflammatory disease is a skin condition such as psoriasis.

The uses of the present invention include, but are not limited to, treatment of diseases for which known TNF antagonists have been shown useful. Three specific TNF antagonists are currently FDA-approved. The drugs are etanercept (Enbrel®), infliximab (Remicade®) and adalimumab (Humira®). One or more of these drugs is approved for the treatment of rheumatoid arthritis, juvenile rheumatoid arthritis, psoriasis, psoriatic arthritis, ankylosing spondylitis, and inflammatory bowel disease (Crohn's disease or ulcerative colitis).

In a preferred embodiment, the receptor is either the TNFR1 or TNFR2 receptors. In other embodiments, the receptor is a member of the TNFR superfamily that is sufficiently homologous to TNFR1 and TNFR2, e.g., TNFRSF3, TNFRSF5, or TNFRSF11A, so that deletion of either or both exons homologous to exons 7 and 8 results in a secreted form. Those skilled in the art appreciate that the operability of the invention is not determined by whether or not such secreted forms are physiological, only that the products of such splice variants are secreted, stable, and capable of ligand-binding.

The administration of the SSO to subjects can be accomplished using procedures developed for ASON. ASON have been successfully administered to experimental animals and human subjects by intravenous administration in saline in doses as high as 6 mg/kg three times a week (Yacysyhn, B. R., et al, 2002, Gut 51:30 (anti-ICAM-1 ASON for treatment of Crohn's disease); Stevenson, J., et al., 1999, J. Clinical Oncology 17:2227 (anti-RAF-1 ASON targeted to PBMC)). The pharmacokinetics of 2′O-MOE phosphorothioate ASON, directed towards TNF-alpha has been reported (Geary, R. S., et al., 2003, Drug Metabolism and Disposition 31:1419). The systemic efficacy of mixed LNA/DNA molecules has also been reported (Fluiter, K., et al., 2003, Nucleic Acids Res. 31:953).

The systemic activity of SSO in a mouse model system was investigated using 2′O-MOE phosphorothioates and PNA chemistries. Significant activity was observed in all tissues investigated except brain, stomach and dermis (Sazani, P., et al., 2002, Nature Biotechnology 20, 1228).

In general any method of administration that is useful in conventional antisense treatments can be used to administer the SSO of the invention. For testing of the SSO in cultured cells, any of the techniques that have been developed to test ASON or SSO may be used.

Formulations of the present invention comprise SSOs in a physiologically or pharmaceutically acceptable carrier, such as an aqueous carrier. Thus formulations for use in the present invention include, but are not limited to, those suitable for parenteral administration including intraperitoneal, intravenous, intraarterial, subcutaneous, or intramuscular injection or infusion, as well as those suitable topical (including ophthalmic and to mucous membranes including vaginal delivery), oral, rectal or pulmonary (including inhalation or insufflation of powders or aerosols, including by nebulizer, intratracheal, intranasal delivery) administration. The formulations may conveniently be presented in unit dosage form and may be prepared by any of the methods well known in the art. The most suitable route of administration in any given case may depend upon the subject, the nature and severity of the condition being treated, and the particular active compound which is being used.

Pharmaceutical compositions of the present invention include, but are not limited to, the physiologically and pharmaceutically acceptable salts thereof, i.e, salts that retain the desired biological activity of the parent compound and do not impart undesired toxicological effects thereto. Examples of such salts are (a) salts formed with cations such as sodium, potassium, NH₄⁺, magnesium, calcium, polyamines such as spermine and spermidine, etc.; (b) acid addition salts formed with inorganic acids, for example, hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, napthalenesulfonic acid, methanesulfonic acid, p-toluenesulfonic acid, napthalenedisulfonic acid, polygalacturonic acid, and the like; and (d) salts formed from elemental anions such as chlorine, bromine, and iodine.

The present invention provides for the use of SSOs having the characteristics set forth above for the preparation of a medicament for increasing the ratio of a soluble form of a TNFR superfamily member to its corresponding membrane bound form, in a patient afflicted with an inflammatory disorder involving excessive activity of a cytokine, such as TNF-α, as discussed above. In the manufacture of a medicament according to the invention, the SSOs are typically admixed with, inter alia, an acceptable carrier. The carrier must, of course, be acceptable in the sense of being compatible with any other ingredients in the formulation and must not be deleterious to the patient. The carrier may be a solid or liquid. SSOs are incorporated in the formulations of the invention, which may be prepared by any of the well known techniques of pharmacy consisting essentially of admixing the components, optionally including one or more accessory therapeutic ingredients.

Formulations of the present invention may comprise sterile aqueous and nonaqueous injection solutions of the active compounds, which preparations are preferably isotonic with the blood of the intended recipient and essentially pyrogen free. These preparations may contain anti-oxidants, buffers, bacteriostats, and solutes which render the formulation isotonic with the blood of the intended recipient. Aqueous and non-aqueous sterile suspensions can include, but are not limited to, suspending agents and thickening agents. The formulations may be presented in unit dose or multi-dose containers, for example, sealed ampoules and vials, and may be stored in freeze-dried (lyophilized) condition requiring only the addition of the sterile liquid carrier, for example, saline or water-for-injection immediately prior to use.

In the formulation the SSOs may be contained within a lipid particle or vesicle, such as a liposome or microcrystal, which may be suitable for parenteral administration. The particles may be of any suitable structure, such as unilamellar or plurilameller, so long as the SSOs are contained therein. Positively charged lipids such as N-[1-(2,3-dioleoyloxi)propyl]-N,N,N-trimethyl-ammoniummethylsulfate, or “DOTAP,” are particularly preferred for such particles and vesicles. The preparation of such lipid particles is well known. [See references in U.S. Pat. No. 5,976,879 col. 6]

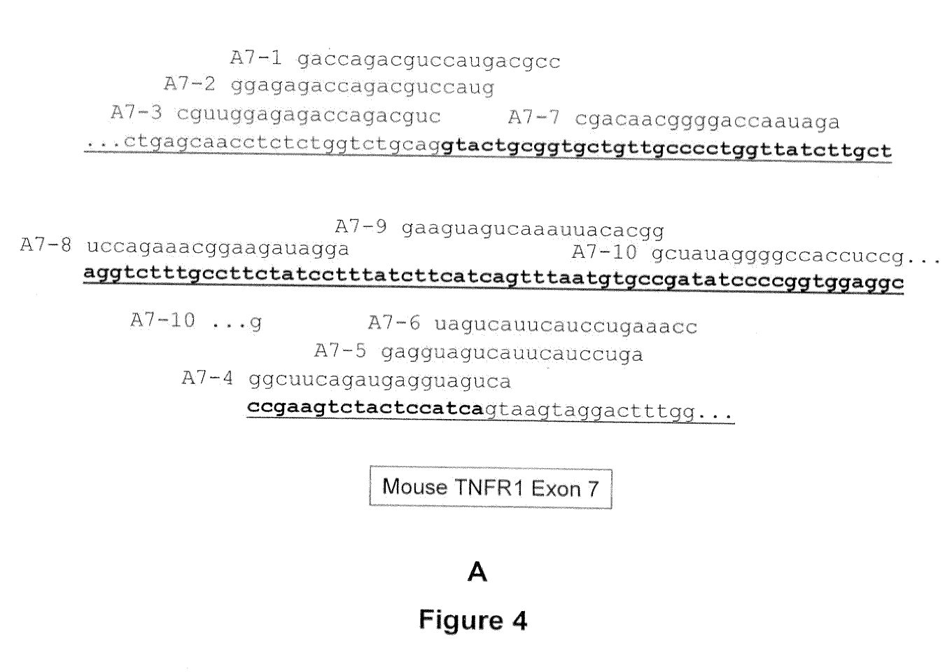

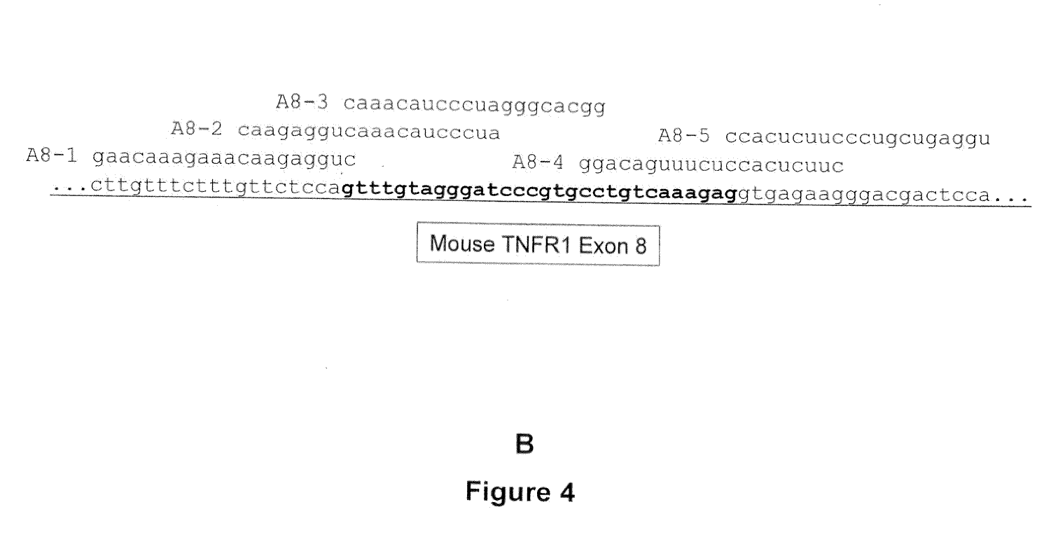

The SSO can be targeted to any element or combination of elements that regulate splicing, including the 3 ′splice site, the 5′ splice site, the branch point, the polypyrimidine tract, exonic splicing enhancers, exonic splicing silencers, intronic splicing enhancers, and intronic splicing silencers. The determination of the sequence of the SSO can be guided by the following tables that shows the activities of the SSOs whose sequences and locations are found as depicted in FIGS. 4, 5, and 8. The person skilled in the art will note that: 1) SSOs complementary to the exon need not be complementary to either the splice acceptor or splice donor sites, note SSOs A7-10, B7-7 and B7-9, Table 1; 2) SSOs complementary to sequences of the intron and as few as one nucleotide of the exon can be operative, note A8-5 and B7-6,

Table 1; 3) SSOs complementary to the intron immediately adjacent to the exon can also be effective, note 3312, Table 2; and 4) efficacy of an oligonucleotide alone is usually predictive of the efficacy of the SSO in combination with other SSOs.

Those skilled in the art can appreciate that the invention as directed toward human TNF-alpha receptors can be practiced using SSO having a sequence that is complementary to at least 10, preferably between 15 and 20 nucleotides of the portions of the TNFR1 or TNFR2 genes comprising exons 7 or 8 and their adjacent introns. It is further preferred that at least one nucleotide of the exon itself is included within the complementary sequence. SEQ ID Nos: 1-4 contain the sequence of Exons 7 and 8 of the TNFR1 (SEQ ID Nos: 1 and 2) and TNFR2 (SEQ ID Nos; 3 and 4) and 50 adjacent nucleotides of the flanking introns. When affinity-enhancing modifications are used, including but not limited to LNA or G-clamp nucleotides, the skilled person recognizes the length of the SSO can be correspondingly reduced. When alternating conventional and LNA nucleotides are used a length of 16 is effective.

Those skilled in the art will also recognize that the selection of SSO sequences must be made with care to avoid self-complementary SSO, which may lead to the formation of partial “hairpin” duplex structures. In addition, high GC content should be avoided to minimize the possibility of non-specific base pairing. Furthermore, SSOs matching off-target genes, as revealed for example by BLAST, should also be avoided.

In some situations, it may be preferred to select an SSO sequence that can target a human and at least one other species. These SSOs can be used to test and to optimize the invention in said other species before being used in humans, thereby being useful for regulatory approval and drug development purposes. For example, SEQ ID Nos: 74, 75, 77, 78, 80, and 89, which target human TNFR2 are also 100% complementary to the corresponding Macaca Mullata sequences. As a result these sequences can be used to test treatments in monkeys, before being used in humans.

It will be appreciated by those skilled in the art that various omissions, additions and modifications may be made to the invention described above without departing from the scope of the invention, and all such modifications and changes are intended to fall within the scope of the invention, as defined by the appended claims. All references, patents, patent applications or other documents cited are herein incorporated by reference.

In the sequence listing below, SEQ ID NOs 1-116 are as disclosed in WO2007/058894. SEQ IDs NOs 117-242 are as disclosed as SEQ ID NOs 1-126 of PCT/US2007/10557. SEQ IDs NOs 243-246 are new to the present application, and are preferred oligomers according to the invention.

TABLE 4
Splice Switching Oligomers targeting human TNFR2:
Capital letters = LNA, small letters = DNA,)-
Note SEQ ID No 243 targets themouse TNFR2.

| | | | | |
|---|---|---|---|---|
| SEQ | | | | |
| ID | Name | Sequence (5′-3′) | Description | Nucleobase Motif |

3378

| | | | | |
|---|---|---|---|---|
| 130 | SK100 | CcA cAa TcA gTc CtA g | 3378 Full Length | CCA CAA TCA GTC |
| | | | | CTA G |
| 131 | SK101 | A cAa TcA gTc CtA g | −2nt 5′ (14mer) | A CAA TCA GTC |
| | | | | CTA G |
| 132 | SK102 | Aa TcA gTc CtA g | −4nt 5′ (12mer) | AA TCA GTC |
| | | | | CTA G |
| 133 | SK103 | TcA gTc CtA g | −6nt 5′ (10mer) | TCA GTC CTA G |
| 134 | SK104 | CcA cAa TcA gTc Ct | −2nt 3′ (14mer) | CCA CAA TCA GTC |
| | | | | CT |
| 135 | SK105 | CcA cAa TcA gTc | −4nt 3′ (12mer) | CCA CAA TCA GTC |
| 136 | SK106 | CcA cAa TcA g | −6nt 3′ (10mer) | CCA CAA TCA G |
| 137 | SK107 | Ca CaA tCa GtC cTa | −1nt 5′ ; −1nt 3′ (14mer) | CA CAA TCA GTC |
| | | | | CTA |
| 138 | SK108 | Ca CaA tCa GtC c | −1nt 5′ ; −3nt 3′ (12mer) | CA CAA TCA GTC |
| | | | | C |
| 139 | SK109 | A cAa TcA gTc Ct | −2nt 5′ ; −2nt 3′ (12mer) | A CAA TCA GTC |
| | | | | CT |
| 140 | SK110 | CaA tCa GtC cTa | −3nt 5′ ; −1nt 3′ (12mer) | CAA TCA GTC |
| | | | | CTA |
| 141 | SK111 | Ca CaA tCa Ct | −1nt 5′ ; −5nt 3′ (10mer) | CA CAA TCA GT |
| 142 | SK112 | A cAa TcA gTc | −2nt 5′ ; −4nt 3′ (10mer) | A CAA TCA GTC |
| 143 | SK113 | CaA tCa GtC c | −3nt 5′ ; −3nt 3′ (10mer) | CAA TCA GTC C |
| 144 | SK114 | Aa TcA gTc Ct | −4nt 5′ ; −2nt 3′ (10mer) | AA TCA GTC CT |
| 145 | SK115 | A tCa GtC cTa | −5nt 5′ ; −1nt 3′ (10mer) | A TCA GTC CTA |

3379

| | | | | |
|---|---|---|---|---|
| 146 | SK116 | CaG tCc TaG aAa GaA | 3379 Full Length | CCA CAA TCA GTC |
| | | a | | CTA G |
| 147 | SK117 | G tCc TaG aAa GaA a | −2nt 5′ (14mer) | G TCC TAG AAA |
| | | | | GAA A |
| 148 | SK118 | Cc TaG aAa GaA a | −4nt 5′ (12mer) | CC TAG AAA GAA |
| | | | | A |
| 149 | SK119 | TaG aAa GaA a | −6nt 5′ (10mer) | TAG AAA GAA A |
| 150 | SK120 | CaG tCc TaG aAa Ga | −2nt 3′ (14mer) | CAG TCC TAG AAA |
| | | | | GA |
| 151 | SK121 | CaG tCc TaG aAa | −4nt 3′ (12mer) | CAG TCC TAG AAA |
| 152 | SK122 | CaG tCc TaG a | −6nt 3′ (10mer) | CAG TCC TAG A |
| 153 | SK123 | Ag TcC tAg AaA gAa | −1nt 5′ ; −1nt 3′ (14mer) | AG TCC TAG AAA |
| | | | | GAA |
| 154 | SK124 | Ag TcC tAg AaA g | −1nt 5′ ; −3nt 3′ (12mer) | AG TCC TAG AAA G |
| 155 | SK125 | G tCc TaG aAa Ga | −2nt 5′ ; −2nt 3′ (12mer) | G TCC TAG AAA GA |
| 156 | SK126 | TcC tAg AaA gAa | −3nt 5′ ; −1nt 3′ (12mer) | TCC TAG AAA GAA |
| 157 | SK127 | Ag TcC tAg Aa | −1nt 5′ ; −5nt 3′ (10mer) | AG TCC TAG AA |
| 158 | SK128 | G tCc TaG aAa | −2nt 5′ ; −4nt 3′ (10mer) | G TCC TAG AAA |
| 159 | SK129 | TcC tAg AaA g | −3nt 5′ ; −3nt 3′ (10mer) | TCC TAG AAA G |
| 160 | SK130 | Cc TaG aAa Ga | −4nt 5′ ; −2nt 3′ (10mer) | CC TAG AAA GA |
| 161 | SK131 | C tAg AaA gAa | −5nt 5′ ; −1nt 3′ (10mer) | C TAG AAA GAA |

3384

| | | | | |
|---|---|---|---|---|
| 162 | SK132 | AcT tTt CaC cTg GgT | 3384 Full Length | CCA CAA TCA GTC |
| | | c | | CTA G |
| 163 | SK133 | T tTt CaC cTg GgT c | −2nt 5′ (14mer) | T TTT CAC CTG |
| | | | | GGT C |
| 164 | SK134 | Tt CaC cTg GgT c | −4nt 5′ (12mer) | TT CAC CTG GGT |
| | | | | C |
| 165 | SK135 | CaC cTg GgT c | −6nt 5′ (10mer) | CAC CTG GGT C |
| 166 | SK136 | AcT tTt CaC cTg Gg | −2nt 3′ (14mer) | ACT TTT CAC CTG |
| | | | | GG |
| 167 | SK137 | AcT tTt CaC cTg | −4nt 3′ (12mer) | ACT TTT CAC CTG |
| 168 | SK138 | AcT tTt CaC c | −6nt 3′ (10mer) | ACT TTT CAC C |
| 169 | SK139 | Ct TtT cAc CtG gGt | −1nt 5′ ; −1nt 3′ (14mer) | CT TTT CAC CTG |
| | | | | GGT |
| 170 | SK140 | Ct TtT cAc CtG g | −1nt 5′ ; −3nt 3′ (12mer) | CT TTT CAC CTG G |
| 171 | SK141 | T tTt CaC cTg Gg | −2nt 5′ ; −2nt 3′ (12mer) | T TTT CAC CTG GG |
| 172 | SK142 | TtT cAc CtG gGt | −3nt 5′ ; −1nt 3′ (12mer) | TTT CAC CTG GGT |
| 173 | SK143 | Ct TtT cAc Ct | −1nt 5′ ; −5nt 3′ (10mer) | CT TTT CAC CT |
| 174 | SK144 | T tTt CaC cTg | −2nt 5′ ; −4nt 3′ (10mer) | T TTT CAC CTG |
| 175 | SK145 | TtT cAc CtG g | −3nt 5′ ; −3nt 3′ (10mer) | TTT CAC CTG G |
| 176 | SK146 | Tt CaC cTg Gg | −4nt 5′ ; −2nt 3′ (10mer) | TT CAC CTG GG |
| 177 | SK147 | T cAc CtG gGt | −5nt 5′ ; −1nt 3′ (10mer) | T CAC CTG GGT |

| | | |
|---|---|---|
| SEQ ID | | |
| No | Sequence | Length |

| | | |
|---|---|---|
| SEQ ID 243 | ^{m}Cₛ^{o}aₛAₛ^{o}tₛ^{m}Cₛ^{o}aₛGₛ^{o}aₛ^{m}Cₛ^{o}cₛTₛ^{o}aₛGₛ^{o}gₛAₛ^{o}a | 16 |
| SEQ ID 244 | ^{m}Cₛ^{o}cₛAₛ^{o} cₛAₛ^{o}aₛTₛ^{o}cₛAₛ^{o}gₛTₛ^{o}cₛ^{m}Cₛ^{o}tₛAₛ^{o}g | 16 |
| SK100 | | |
| SEQ ID 245 | ^{m}Cₛ^{o}aₛ^{m}Cₛ^{o}aₛAₛ^{o}tₛ^{m}Cₛ^{o}aₛGₛ^{o}tₛ^{m}Cₛ^{o}cₛTₛ^{o}a | 14 |
| SK107 | | |
| SEQ ID 246 | Aₛ^{o}cₛAₛ^{o}aₛTₛ^{o}cₛAₛ^{o}gₛTₛ^{o}cₛ^{m}Cₛ^{o}t | 12 |
| SK109 | | |
| SEQ ID 251 | Aₛ^{o}cₛAₛ^{o}aₛTₛ^{o}c ₛAₛ^{o}gₛTₛ^{o}cₛ^{m}Cₛ^{o}tₛAₛ^{o}g | 14 |
| SK101 | | |
| SEQ ID 252 | Aₛ^{o}aₛTₛ^{o}cₛAₛ^{o}gₛTₛ^{o}cₛ^{m}Cₛ^{o}tₛAₛ^{o}g | 12 |
| SK102 | | |
| SEQ ID 253 | Tₛ^{o}cₛAₛ^{o}ₛTₛ^{o}cₛ^{m}Cₛ^{o}tₛAₛ^{o}g | 10 |
| SK103 | | |
| SEQ ID 254 | ^{m}Cₛ^{o}cₛAₛ^{o}cₛAₛ^{o}aₛTₛ^{o}cₛAₛ^{o}gₛTₛ^{o}cₛ^{m}Cₛ^{o}t | 14 |
| SK104 | | |
| SEQ ID 255 | ^{m}Cₛ^{o}cₛAₛ^{o}cₛAₛ^{o}aₛTₛ^{o}cₛAₛ^{o}gₛTₛ^{o}c | 12 |
| SK105 | | |
| SEQ ID 256 | ^{m}Cₛ^{o}cₛAₛ^{o}cₛAₛ^{o}aₛTₛ^{o}cₛAₛ^{o}g | 10 |
| SK106 | | |
| SEQ ID 257 | ^{m}Cₛ^{o}aₛ^{m}Cₛ^{o}aₛAₛ^{o}tₛ^{m}Cₛ^{o}aₛGₛ^{o}tₛ^{m}Cₛ^{o}c | 12 |
| SK108 | | |
| SEQ ID 258 | ^{m}Cₛ^{o}aₛAₛ^{o}tₛ^{m}Cₛ^{o}aₛGₛ^{o}tₛ^{m}Cₛ^{o}cₛTₛ^{o}a | 12 |
| SK110 | | |
| SEQ ID 259 | ^{m}Cₛ^{o}aₛ^{m}Cₛ^{o}aₛAₛ^{o}tₛ^{m}Cₛ^{o}aₛGₛ^{o}t | 10 |
| SK111 | | |
| SEQ ID 260 | Aₛ^{o}cₛAₛ^{o}aₛTₛ^{o}cₛAₛ^{o}gₛTₛ^{o}c | 10 |
| SK112 | | |
| SEQ ID 261 | ^{m}Cₛ^{o}aₛAₛ^{o}tₛ^{m}Cₛ^{o}aₛGₛ^{o}tₛ^{m}Cₛ^{o}c | 10 |
| SK113 | | |
| SEQ ID 262 | Aₛ^{o}aₛTₛ^{o}cₛAₛ^{o}gₛtₛ^{o}cₛ^{m}Cₛ^{o}t | 10 |
| SK114 | | |
| SEQ ID 263 | Aₛ^{o}tₛ^{m}Cₛ^{o}aₛGₛ^{o}tₛ^{m}Cₛ^{o}cₛTₛ^{o}a | 10 |
| SK115 | | |
| SEQ ID 264 | ^{m}Cₛ^{o}aₛGₛ^{o}tₛ^{m}Cₛ^{o}cₛTₛ^{o}aₛGₛ^{o}aₛAₛ^{o}aₛGₛ^{o}aₛAₛ^{o}a | 16 |
| SK116 | | |
| SEQ ID 265 | Gₛ^{o}tₛ^{m}Cₛ^{o}cₛTₛ^{o}aₛGₛ^{o}aₛAₛ^{o}aₛGₛ^{o}aₛAₛ^{o}a | 14 |
| SK117 | | |
| SEQ ID 266 | ^{m}Cₛ^{o}cₛTₛ^{o}aₛG_{S}^{o}aₛAₛ^{o}aₛGₛ^{o}aₛAₛ^{o}a | 12 |
| SK118 | | |
| SEQ ID 267 | Tₛ^{o}aₛGₛ^{o}aₛAₛ^{o}aₛGₛ^{o}aₛAₛ^{o}a | 10 |
| SK119 | | |
| SEQ ID 268 | ^{m}Cₛ^{o}aₛGₛ^{o}tₛ^{m}Cₛ^{o}cₛTₛ^{o}aₛGₛ^{o}aₛAₛ^{o}aₛGₛ^{o}a | 14 |
| SK120 | | |
| SEQ ID 269 | ^{m}Cₛ^{o}aₛGₛ^{o}tₛ^{m}Cₛ^{o}cₛb Tₛ^{o}aₛGₛ^{o}aₛAₛ^{o}a | 12 |
| SK121 | | |
| SEQ ID 270 | ^{m}Cₛ^{o}aₛGₛ^{o}tₛ^{m}Cₛ^{o}cₛTₛ^{o}aₛGₛ^{o}a | 10 |
| SK122 | | |
| SEQ ID 271 | Aₛ^{o}gₛTₛ^{o}cₛ^{m}Cₛ^{o}tₛAₛ^{o}gₛAₛ^{o}aₛAₛ^{o}gₛAₛ^{o}a | 14 |
| SK123 | | |
| SEQ ID 272 | Aₛ^{o}gₛTₛ^{o}cₛ^{m}Cₛ^{o}tₛAₛ^{o}gₛAₛ^{o}aₛAₛ^{o}g | 12 |
| SK124 | | |
| SEQ ID 273 | Gₛ^{o}tₛ^{m}Cₛ^{o}cₛTₛ^{o}aₛGₛ^{o}aₛAₛ^{o}aₛGₛ^{o}a | 12 |
| SK125 | | |
| SEQ ID 274 | Tₛ^{o}cₛ^{m}Cₛ^{o}tₛAₛ^{o}gₛAₛ^{o}gₛAₛ^{o}a | 12 |
| SK126 | | |
| SEQ ID 275 | Aₛ^{o}gₛTₛ^{o}cₛ^{m}Cₛ^{o}tₛAₛ^{o}gₛAₛ^{o}a | 10 |
| SK127 | | |
| SEQ ID 276 | Gₛ^{o}tₛ^{m}Cₛ^{o}cₛTₛ^{o}sₛGₛ^{o}aₛAₛ^{o}a | 10 |
| SK128 | | |
| SEQ ID 277 | Tₛ^{o}cₛ^{m}Cₛ^{o}tₛAₛ^{o}gₛAₛ^{o}aₛAₛ^{o} g | 10 |
| SK129 | | |
| SEQ ID 278 | ^{m}Cₛ^{o}cₛTₛ^{o}aₛGₛ^{o}aₛAₛ^{o}aₛGₛ^{o}a | 10 |
| SK130 | | |
| SEQ ID 279 | ^{m}Cₛ^{o}tₛAₛ^{o}gₛAₛ^{o}aₛAₛ^{o}gₛAₛ^{o}a | 10 |
| SK131 | | |
| SEQ ID 280 | Aₛ^{o}cₛTₛ^{o}tₛTₛ^{o}tₛ^{m}Cₛ^{o}aₛ^{m}Cₛ^{o}cₛTₛ^{o}gₛGₛ^{o}gₛTₛ^{o}c | 16 |
| SK132 | | |
| SEQ ID 281 | Tₛ^{o}tₛTₛ^{o}tₛ^{m}Cₛ^{o}aₛ^{m}Cₛ^{o}cₛTₛ^{o}gₛGₛ^{o}gₛTₛ^{o}c | 14 |
| SK133 | | |
| SEQ ID 282 | Tₛ^{o}tₛ^{m}Cₛ^{o}aₛ^{m}Cₛ^{o}cₛTₛ^{o}gₛGₛ^{o}gₛTₛ^{o}c | 12 |
| SK134 | | |
| SEQ ID 283 | ^{m}Cₛ^{o}aₛ^{m}Cₛ^{o}Cₛ^{o}cₛTₛ^{o}gₛGₛ^{o}gₛTₛ^{o}c | 10 |
| SK135 | | |
| SEQ ID 284 | Aₛ^{o}cₛTₛ^{o}tₛTₛ^{o}tₛ^{m}Cₛ^{o}aₛ^{m}Cₛ^{o}cₛTₛ^{o}gₛGₛ^{o}g | 14 |
| SK136 | | |
| SEQ ID 285 | Aₛ^{o}cₛTₛ^{o}tₛTₛ^{o}tₛ^{m}Cₛ^{o}aₛ^{m}Cₛ^{o}cₛTₛ^{o}g | 12 |
| SK137 | | |
| SEQ ID 286 | Aₛ^{o}cₛTₛ^{o}tₛTₛ^{o}tₛ^{m}Cₛ^{o}aₛ^{m}Cₛ^{o}c | 10 |
| SK138 | | |
| SEQ ID 287 | ^{m}Cₛ^{o}tₛTₛ^{o}tₛTₛ^{o}cₛAₛ^{o}cₛ^{m}Cₛ^{o}tₛGₛ^{o}gₛGₛ^{o}t | 14 |
| SK139 | | |
| SEQ ID 288 | ^{m}Cₛ^{o}tₛTₛ^{o}tₛTₛ^{o}cₛAₛ^{o}cₛ^{m}Cₛ^{o}tₛGₛ^{o}g | 12 |
| SK140 | | |
| SEQ ID 289 | Tₛ^{o}tₛTₛ^{o}tₛ^{m}Cₛ^{o}aₛ^{m}Cₛ^{o}cₛTₛ^{o}gₛGₛ^{o}g | 12 |
| SK141 | | |
| SEQ ID 290 | Tₛ^{o}tₛTₛ^{o}cₛAₛ^{o}cₛ^{m}Cₛ^{o}tₛGₛ^{o}gₛGₛ^{o}t | 12 |
| SK142 | | |
| SEQ ID 291 | ^{m}Cₛ^{o}tₛTₛ^{o}tₛTₛ^{o}cₛAₛ^{o}cₛ^{m}Cₛ^{o}t | 10 |
| SK143 | | |
| SEQ ID 292 | Tₛ^{o}tₛTₛ^{o}tₛ^{m}Cₛ^{o}aₛ^{m}Cₛ^{o}cₛTₛ^{o}g | 10 |
| SK144 | | |
| SEQ ID 293 | Tₛ^{o}tₛTₛ^{o}cₛAₛ^{o}cₛ^{m}Cₛ^{o}tₛGₛ^{o}g | 10 |
| SK145 | | |
| SEQ ID 294 | Tₛ^{o}tₛ^{m}Cₛ^{o}aₛ^{m}Cₛ^{o}cₛTₛ^{o}gₛGₛ^{o}g | 10 |
| SK146 | | |
| SEQ ID 295 | Tₛ^{o}cₛAₛ^{o}cₛ^{m}Cₛ^{o}tₛGₛ^{o}gₛGₛ^{o}t | 10 |
| SK147 | | |

Capital letters = LNA, preferably oxy LNA (superscript o), preferably phosphorothioate linkages = subscript s, small letters 32 DNA). ^{m}C prefcrably, 5-methylcytosine.

Further embodiments of the invention:

The invention provides for a method of treating an inflammatory disease or condition which comprises administering one or more splice switching oligomers (SSOs) to a subject for a time and in an amount to reduce the activity of a ligand for a receptor of the tumor necrosis factor receptor (TNFR) superfamily, wherein said one or more SSOs are capable of altering the splicing of a pre-mRNA encoding said receptor to increase production of a stable, secreted, ligand-binding form of said receptor.

In one embodiment the mammalian receptor selected from the group consisting of TNFRSF1A, TNFRSF1B, TNFRSF3, TNFRSF5, TNFRSF8, and TNFRSF11A.

In one embodiment the receptor is a human TNFRSF1A or a human TNFRSF1B.

In one embodiment the receptor is a human TNFRSF1B.

In one embodiment the ligand is TNF-.alpha., RANKL, CD40L, LT-.alpha., or LT-.beta.

In one embodiment the disease or condition is selected from the group consisting of rheumatoid arthritis, juvenile rheumatoid arthritis, psoriasis, psoriatic arthritis, ankylosing spondylitis, inflammatory bowel disease (Crohn's disease or ulcerative colitis), hepatitis, sepsis, alcoholic liver disease, and non-alcoholic steatosis.

In one embodiment of the method of treating an inflammatory disease or condition the two or more SSOs are administered.

In one embodiment the receptor is TNFRSF1A, TNFRSF1B, TNFRSF3, TNFRSF5, or TNFRSF11A, and said altering the splicing of said pre-mRNA comprises excising exon 7, exon 8, or both from said pre-mRNA.

In one embodiment said altering the splicing of said pre-mRNA comprises excising exon 7.

In one embodiment said receptor is a human TNFRSF1A or a human TNFRSF1B, and said SSO comprises from at least 10 to at least 20 nucleotides which are complementary to a contiguous sequence from SEQ ID Nos: 1, 2, 3 or 4.

In one embodiment the sequence of said SSO comprises a sequence selected from the group consisting of SEQ ID Nos: 74, 75, 77, 73, 80, 82, 84, and 86-89.

In one embodiment said SSOs comprise one or more nucleotides or nucleosides independently selected from the group consisting of 2′-deoxyribonucleotides, 2′O-Me ribonucleotides, 2′O-MOE ribonucleotides, hexitol (HNA) nucleotides or nucleosides, 2′O-4′C-linked bicyclic ribofuranosyl (LNA) nucleotides or nucleosides, phosphorothioate analogs of any of the foregoing, peptide nucleic acid (PNA) analogs of any of the foregoing; methylphosphonate analogs of any of the foregoing, peptide nucleic acid analogs of any of the foregoing N3′.fwdarw.P5′ phosphoramidate analogs of any of the foregoing, and phosphorodiamidate morpholino nucleotide analogs of any of the foregoing, and combinations thereof.

In one embodiment said SSOs comprise one or more nucleotides or nucleosides independently selected from the group consisting of 2′O-Me ribonucleotides and 2′O-4′C-linked bicyclic ribofuranosyl (LNA) nucleotides or nucleosides.

In one embodiment said administration is parenteral, topical, oral, rectal, or pulmonary.

In one embodiment the invention provides for a method of increasing the production of a stable, secreted, ligand-binding form of a receptor from the TNFR superfamily in a cell, which comprises administering one or more splice switching oligomers (SSOs) to said cell, wherein said one or more SSOs are capable of altering the splicing of a pre-mRNA encoding said receptor to increase production of a stable, secreted, ligand-binding form of said receptor.

In one embodiment the method is performed in vivo.

In one embodiment said receptor is a mammalian receptor selected from the group consisting of TNFRSF1A, TNFRSF1B, TNFRSF3, TNFRSF5, TNFRSF8, and TNFRSF1A.

In one embodiment said receptor is a human TNFRSF1A or a human TNFRSF1B.

In one embodiment said receptor is a human TNFRSF1B.

In one embodiment said SSO comprises from at least 10 to at least 20 nucleotides which are complementary to a contiguous sequence from SEQ ID Nos: 1, 2, 3 or 4.

In one embodiment the invention provides for a splice switching oligomer (SSO) comprising from at least 10 to at least 20 nucleotides, said SSO capable of altering the splicing of a pre-mRNA encoding a receptor from the TNFR superfamily to produce a stable, secreted, ligand-binding form of said receptor.

In one embodiment said receptor is a mammalian receptor selected from the group consisting of TNFRSF1A, TNFRSF1B, TNFRSF3, TNFRSF5, TNFRSF8, and TNFRSF11A.

In one embodiment said receptor is a human TNFSF1A or a human TNFRSF1B.

In one embodiment said receptor is a human TNFRSF1B.

In one embodiment the SSO comprises from at least 10 to at least 20 nucleotides which are complementary to a contiguous sequence from SEQ ID Nos: 1, 2, 3 or 4.

In one embodiment the SSO comprises one or more nucleotides or nucleosides independently selected from the group consisting of 2′-deoxyribonucleotides, 2′O-Me ribonucleotides, 2′O-MOE ribonucleotides, hexitol (HNA) nucleotides or nucleosides, 2′O-4′C-linked bicyclic ribofuranosyl (LNA) nucleotides or nucleosides, phosphorothioate analogs of any of the foregoing, peptide nucleic acid (PNA) analogs of any of the foregoing; methylphosphonate analogs of any of the foregoing, peptide nucleic acid analogs of any of the foregoing, N3′.fwdarw.P5′ phosphoramidate analogs of any of the foregoing, and phosphorodiamidate morpholino nucleotide analogs of any of the foregoing, and combinations thereof.

In one embodiment said 2′O-4′C-linked bicyclic ribofuranosyl (LNA) nucleotides or nucleosides are 2′O-4′C-(methylene)-ribofuranosyl nucleotides or nucleosides, respectively, or 2′O-4′C-(ethylene)-ribofuranosyl nucleotides or nucleosides, respectively.

In one embodiment said SSOs comprise one or more nucleotides or nucleosides independently selected from the group consisting of 2′O-Me ribonucleotides and 2′O-4′-linked bicyclic ribofuranosyl (LNA) nucleotides or nucleosides.

In one embodiment the sequence of said SSO comprises a sequence selected from the group consisting of SEQ ID Nos: 8, 9, 14, 17-21, 24-29, 32, 33, 38-42, 44-46, 50-52, 55-57, 60, 68-71, 74, 75, 77, 78, 80, 82, 84, and 86-89.

In one embodiment the invention provides a pharmaceutical composition comprising the SSO and a pharmaceutically acceptable carrier.

In one embodiment said SSO comprises from at least 10 to at least 20 nucleotides which are complementary to a contiguous sequence from SEQ ID Nos: 1, 2, 3 or 4.

In one embodiment the invention provides an isolated protein capable of binding tumor necrosis factor (TNF), said protein having a sequence comprising the amino acids encoded by a cDNA derived from a mammalian tumor necrosis factor receptor (TNFR) gene, wherein the cDNA comprises in 5′ to 3′ contiguous order, the codon encoding the first amino acid after the cleavage point of the signal sequence of said gene through exon 6 of said gene and exon 8 of said gene through exon 10 of said gene; or the codon encoding the first amino acid of the open reading frame of said gene through exon 6 of said gene and exon 8 of said gene through exon 10 of said gene.

In one embodiment said TNF is TNF-α.

In one embodiment said protein contains at least one processing, chemical, or post-translational modification, and wherein said modification is selected from the group consisting of acetylation, acylation, amidation, ADP-ribosylation, glycosylation, methylation, pegylation, prenylation, phosphorylation, or cholesterol conjugation.

In one embodiment said receptor is TNFR1, such as human TNFR1, In one embodiment, said receptor is TNFR2, such as human TNFR1, In one embodiment said protein comprises a sequence selected from the group consisting of SEQ ID No: 6, amino acids 30-417 of SEQ ID No: 6, SEQ ID No: 8, amino acids 30-416 of SEQ ID No: 8, SEQ ID No: 10, amino acids 23-435 of SEQ ID No: 10, SEQ ID No: 12, and amino acids 23-448 of SEQ ID No: 12. In one embodiment, the invention provides a pharmaceutical composition comprising the protein according to the invention in admixture with a pharmaceutically acceptable carrier. In one embodiment, the invention provides a composition comprising the purified protein according to the invention.

In one embodiment, the invention provides a method of treating an inflammatory disease or condition which comprises administering the pharmaceutical composition according to the invention a subject for a time and in an amount effective to reduce the activity of TNF. In one embodiment, said disease or condition is selected from the group consisting of rheumatoid arthritis, juvenile rheumatoid arthritis, psoriasis, psoriatic arthritis, ankylosing spondylitis, inflammatory bowel disease (Crohn's disease or ulcerative colitis), hepatitis associated with hepatitis A virus, hepatitis associated with hepatitis B virus, hepatitis associated with hepatitis C virus, hepatitis associated with ischemia/reperfusion, sepsis, alcoholic liver disease, and non-alcoholic steatosis. In one embodiment, the invention provides an isolated nucleic acid derived from a mammalian tumor necrosis factor receptor (TNFR) gene and encoding a protein capable of binding tumor necrosis factor (TNF), wherein the cDNA of said protein comprises in 5′ to 3′ contiguous order, the codon encoding the first amino acid after the cleavage point of the signal sequence of said gene through exon 6 of said gene and exon 8 of said gene through exon 10 of said gene; or the codon encoding the first amino acid of the open reading frame of said gene through exon 6 of said gene and exon 8 of said gene through exon 10 of said gene. In such as embodiment, the sequence of said protein comprises a sequence selected from the group consisting of SEQ ID No: 6, amino acids 30-417 of SEQ ID No: 6, SEQ ID No: 8, amino acids 30-416 of SEQ ID No: 8, SEQ ID No: 10, amino acids 23-435 of SEQ ID No: 10, SEQ ID No: 12, and amino acids 23-448 of SEQ ID No: 12. In one embodiment, the sequence of said nucleic acid comprises a sequence selected from the group consisting of nucleotides 1-1251 of SEQ ID No: 5, nucleotides 88-1251 of SEQ ID No: 5, nucleotides 1-1248 of SEQ ID No: 7, nucleotides 88-1248 of SEQ ID No: 7, nucleotides 1-1305 of SEQ ID No: 9, nucleotides 67-1305 of SEQ ID No: 9, nucleotides 1-1344 of SEQ ID No: 11, and nucleotides 67-1344 of SEQ ID No: 11. In one embodiment, the invention provides for an expression vector comprising the nucleic acid of the invention operably linked to a regulatory sequence.

In one embodiment, the invention provides a method of increasing the level of a TNF antagonist in a mammal which comprises transforming cells of said mammal with the expression vector according to the invention to thereby express said TNF antagonist, wherein said vector drives expression of said TNFR.

In one embodiment the mammal is a human, such as a human is an individual having an inflammatory disease or condition.

In one embodiment said expression vector is a plasmid, or a virus.

In one embodiment cells are transformed in vivo.

In one embodiment cells are transformed ex vivo.

In one embodiment, said expression vector comprises a tissue specific promoter—said tissue specific promoter may, for example be derived from a hepatocyte or a macrophage.

In one embodiment the cells are selected from the group consisting of hepatocytes, hematopoietic cells, spleen cells, and muscle cells.

The invention provides for a cell transformed with the expression vector of the invention, such as a mammalian cell, an insect cell, or a microbial cell.

The invention provides for a process for producing a protein capable of binding tumor necrosis factor (TNF) which comprises culturing the cell of the invention under conditions suitable to express said protein, and recovering said protein.

The invention provides for a pharmaceutical composition comprising the nucleic acid or vector of the invention, in admixture with a pharmaceutically acceptable carrier.

The invention provides a method of treating an inflammatory disease or condition which comprises administering the expression vector of the invention to a subject for a time and in an amount sufficient to reduce TNF activity, such as TNF-α activity.

In one embodiment, disease or condition is selected from the group consisting of rheumatoid arthritis, juvenile rheumatoid arthritis, psoriasis, psoriatic arthritis, ankylosing spondylitis, inflammatory bowel disease (Crohn's disease or ulcerative colitis), hepatitis associated with hepatitis A virus, hepatitis associated with hepatitis B virus, hepatitis associated with hepatitis C virus, hepatitis associated with ischemia/reperfusion, sepsis, alcoholic liver disease, and non-alcoholic steatosis.

In one embodiment, the invention provides for a method of treating an inflammatory disease or condition which comprises administering one or more splice switching oligomers (SSOs) to a subject for a time and in an amount to reduce the activity of TNF, wherein said one or more SSOs are capable of altering the splicing of a pre-mRNA encoding a mammalian tumor necrosis factor receptor 2 (TNFR2) (or TNFR1) to increase production of a protein capable of binding tumor necrosis factor (TNF), wherein said protein has a sequence comprising the amino acids encoded by a cDNA derived from a gene for said receptor, wherein the cDNA comprises in 5′ to 3′ contiguous order, the codon encoding the first amino acid after the cleavage point of the signal sequence of said gene through exon 6 of said gene and exon 8 of said gene through exon 10 of said gene; or the codon encoding the first amino acid of the open reading frame of said gene through exon 6 of said gene and exon 8 of said gene through exon 10 of said gene.

In one embodiment, said disease or condition is selected from the group consisting of rheumatoid arthritis, juvenile rheumatoid arthritis, psoriasis, psoriatic arthritis, ankylosing spondylitis, inflammatory bowel disease (Crohn's disease or ulcerative colitis), hepatitis associated with hepatitis A virus, hepatitis associated with hepatitis B virus, hepatitis associated with hepatitis C virus, hepatitis associated with ischemia/reperfusion, sepsis, alcoholic liver disease, and non-alcoholic steatosis. In one embodiment, the administration is parenteral, topical, oral, rectal, or pulmonary.

In one embodiment, the invention provides a splice switching oligomer (SSO) comprising at least 8 nucleotides, said SSO capable of altering the splicing of a pre-mRNA encoding a mammalian tumor necrosis factor receptor 2 (TNFR2) (or TNFR1) to produce a protein capable of binding tumor necrosis factor (TNF), wherein said protein has a sequence comprising the amino acids encoded by a cDNA derived from a gene for said receptor, wherein the cDNA comprises in 5′ to 3′ contiguous order, the codon encoding the first amino acid after the cleavage point of the signal sequence of said gene through exon 6 of said gene and exon 8 of said gene through exon 10 of said gene; or the codon encoding the first amino acid of the open reading frame of said gene through exon 6 of said gene and exon 8 of said gene through exon 10 of said gene.

In one embodiment, the invention provides for a SSO which comprises at least 8 nucleotides which are complementary to a contiguous sequence from SEQ ID No: 13.

In one embodiment the sequence of said SSO comprises a sequence selected from the group consisting of SEQ ID Nos: 14, 30, 46, 70, 71, 72, and 73, and subsequences thereof at least 8 nucleotides.

In one embodiment the sequence of said SSO comprises a sequence selected from the group consisting of SEQ ID Nos: 14-61.

The invention provides for a method of increasing the production of a protein capable of binding tumor necrosis factor (TNF), in a cell, which comprises administering one or more splice switching oligomers (SSOs) to said cell, wherein said protein has a sequence comprising the amino acids encoded by a cDNA derived from a mammalian tumor necrosis factor receptor 2 (TNFR2) (or TNFR1) gene, wherein the cDNA comprises in 5′ to 3′ contiguous order, the codon encoding the first amino acid after the cleavage point of the signal sequence of said gene through exon 6 of said gene and exon 8 of said gene through exon 10 of said gene; or the codon encoding the first amino acid of the open reading frame of said gene through exon 6 of said gene and exon 8 of said gene through exon 10 of said gene, and wherein said one or more SSOs are capable of altering the splicing of a pre-mRNA encoding said receptor to increase production of said protein. In one embodiment, the method is performed in vivo.

The invention provides for a pharmaceutical composition comprising the SSO of the invention and a pharmaceutically acceptable carrier.

### EXAMPLES

The following Examples are identical to those described in PCT/US2006/043651 (Examples 1-11) and PCT/US2007/10557 (Examples 12-44), although reference numbers have been rearranged accordingly.

### Example 1

### Materials and Methods

Oligonucleotides. All uniformly modified 2′-O-methyl-ribonucleoside-phosphorothioate (2′-OMe) 20-mers were synthesized by Trilink Biotechnologies, San Diego, Calif. Their sequences are listed in Table 1. Tables 2 and 3 show the sequences of chimeric LNA SSOs with alternating 2′deoxy- and 2′O-4′-(methylene)-bicyclic-ribonucleoside phosphorothioates. These were synthesized by Santaris Pharma, Denmark. For each LNA oligonucleotide, the 5′-terminal nucleoside was a 2′O-4′-methylene-ribonucleoside and the 3′-terminal ribonucleoside was a 2 ′deoxy-ribonucleoside.

Cell culture and transfections. NIH-3T3 cells were maintained (37° C., 5% CO₂) in Dulbecco's modified Eagle's media (DMEM) supplemented with 10% Colorado fetal calf serum and antibiotic. L929 cells were maintained (37° C., 5% CO₂) in minimal essential media supplemented with 10% fetal bovine serum and antibiotic. For transfection, either NIH-3T3 or L929 cells were seeded in 24-well plates at 10⁵ cells per well and transfected 24 hours later.

Oligonucleotides were complexed, at the indicated concentrations, with 2 μL of Lipofectamine™ 2000 transfection reagent (Invitrogen) as per the manufacturer's directions. The nucleotide/lipid complexes were then applied to the cells and incubated for hours. The media was then aspirated and cells harvested with TRI-Reagent™ (MRC, Cincinnati, Ohio).

RT-PCR. Total RNA was isolated with TRI-Reagent (MRC, Cincinnati, Ohio) and TNFR1 or TNFR2 mRNA was amplified by RT-PCR using rTth polymerase (Applied Biosystems) following supplier directions. Murine TNFR1 mRNA was amplified using forward primer PS009 (SEQ ID No: 111) (5′-GAA AGT GAG TGC GTC CCT TGC-3′) and reverse primer PS010 (SEQ ID No: 112) (5′-GCA CGG AGC AGA GTG ATT CG-3′). Murine TNFR2 mRNA was amplified using forward primer PS003 (SEQ ID No: 113) (5′-GAG CCC CAA ATG GAA ATG TGC-3) and reverse primer PS004 (SEQ ID No: 114) (5′-GCT CAA GGC CTA CTG CC-3′). Human TNFR2 mRNA was amplified using forward primer (SEQ ID No: 115) (5′-ACT GAA ACA TCA GAC GTG GTG TGC-3′) and reverse primer (SEQ ID No: 116) (5′-CCT TAT CGG CAG GCA AGT GAG-3′). A Cy5-labeled dCTP (GE Healthcare) was included in the PCR step for visualization (0.1 μL per 50 μL PCR reaction). Cycles of PCR proceeded: 95° C., 60 see; 56° C., 30 see; 72° C., 60 sec for 22 cycles total. The PCR products were separated on a 10% non-denaturing polyacrylamide gel, and Cy5-labeled bands were visualized with a Typhoon™ 9400 Scanner (GE Healthcare). Scans were quantified with ImageQuant™ (GE Healthcare) software.

Mouse hepatocyte cultures. For hepatocyte collection, livers of mice were perfused with RPMI medium containing 0.53 mg/ml of collagenase (Worthington Type 1, code CLS). After perfusion, the cell suspension was collected and seeded in a stop solution of RPMI with 10% (vol/vol) FBS and 0.5% penicillin-streptomycin plus 1 nM insulin and 13 nM dexamethASONe. Approximately 3×10⁵ cells were seeded on a six-well collagen-coated plate. The seeding medium was replaced 1 hour later with maintenance medium consisting of seeding medium without the 10% (vol/vol) FBS. Varying amounts of oligonucleotide-lipid complexes were applied 24 hours later. Cells were lysed 24 hours after transfection with TRI-Reagent™.

Human hepatocyte cultures. Human hepatocytes were obtained in suspension either from ADMET technologies, or from The UNC Cellular Metabolism and Transport Core at UNC-Chapel Hill. Cells were washed and suspended in RPMI 1640 supplemented with 10% FBS, 1 μg/ml human insulin, and 13 nM DexamethASONe. Hepatocytes were plated in 6-well plates at 0.5×10⁶ cells per plate in 3 mL media. After 1-1.5 hours, non-adherent cells were removed, and the media was replaced with RPMI 1640 without FBS, supplemented with 1 microg/ml human insulin, and 130 nM DexamethASONe.

For delivery of LNA SSOs to hepatocytes in 6-well plates, 10 μL of a 5 μM LNA stock was diluted into 100 μL of OPTI-MEM™, and 4 μL of Lipofectamine™ 2000 was diluted into 100 μL of OPTI-MEM™. The 200 μL complex solution was then applied to the cells in the 6-well plate containing 2800 μL of media, for a total of 3000 μL. The final LNA concentration was 17 nM. After 24 hours, cells were harvested in TRI-Reagent™. Total RNA was isolated per the manufacturers directions. Approximately 200 ng of total RNA was subjected to reverse transcription-PCR (RT-PCR).

ELISA. To determine the levels of soluble TNFR2 in cell culture media or mouse sera, the Quantikine® Mouse sTNF RII ELISA kit from R&D Systems (Minneapolis, Minn.) was used. To determine the levels of soluble TNFR1 in cell culture media or mouse sera, the Quantikine® Mouse sTNF RI ELISA kit from R&D Systems (Minneapolis, Minn.) was used. Note, the antibodies used for detection also detect the protease cleavage forms of the receptor.

For cell culture studies, extracellular media was collected at 72 hours post transfection. The assay was performed according to the manufacturer's guide, using 50 μL of undiluted media. The assay readings were performed using a microplate reader set at 450 nm, with wavelength correction set at 570 nm.

For mouse in vivo studies, blood from the animals was clotted for 1 hour at 37° C. and centrifuged for 10 min at 14,000 rpm (Jouan BRA41 centrifuge). Sera was collected and assayed according to the manufacturer's guide, using 50 μL of mouse sera, diluted 1:10. The assay readings were performed using a microplate reader set at 450 nm, with wavelength correction set at 570 mm.

L929 cytotoxicity assay. L929 cells plated in 96-well plates at 10⁴ cells per plate were treated with 0.1 ng/mL TNF-alpha (TNF) and actinomycin D (ActD) in the presence of 10% serum from mice treated with the indicated oligonucleotide in 100 μL total cell culture media. Control lanes were plated in 10% serum from untreated mice. 24 hours later, cell viability was measured by adding 20 μL CellTiter 96® Aqueous Solution (Promega) and measuring absorbance at 490 nm with a microplate reader. Cell viability was normalized to cells untreated with TNF/ActD.

### Example 2

### Testing of SSOs for Splice Switching Activity

SSOs were synthesized, transfected into either NIH-3T3 or L929 cells. Total RNA from the cells was analyzed by RT-PCR to assess the splice switching ability of the SSO. Table 1 contains the sequences and the splice switching activities of 20 nucleotide 2′O-Me-ribonucleoside-phosphorothioate murine SSOs. Table 2 contains the sequences and the splice switching activities of 16 nucleotide chimeric INA murine SSOs. Table 3 contains the sequences and the splice switching activities of 16 nucleotide chimeric LNA human SSOs. Each table also lists the target site for each SSO by complementary regions and number of nucleotides; e.g., 16:E7(8:8) means complementary to the 3′-most 8 nucleotides of intron 6 and the 5′-most 8 nucleotides of exon 7; E7(16) means complementary to 16 nucleotides in exon 7; and E8:18(7:9) means complementary to the 3′-most 7 nucleotides of exon 8 and the 5′-most 9 nucleotides of intron 8.

TABLE 1
2′O-Me-ribonucleoside-phosphorothioate mouse targeted SSO

| | | | | |
|---|---|---|---|---|
| SEQ ID. | Name* | Sequence (5′-3′) | Activity | Target Site |

| | | | | |
|---|---|---|---|---|
| 5 | A7-1 | CCG CAG UAC CUG CAG ACC AG | − | 16:E7(6:14) |
| 6 | A7-2 | GUA CCU GCA GAC CAG AGA GG | − | 16:E7(13:7) |
| 7 | A7-3 | CUG CAG ACC AGA GAG GUU GC | − | 16:E7(18:2) |
| 8 | A7-4 | ACU GAU GGA GUA GAC UUC GG | + | E7:17(18:2) |
| 9 | A7-5 | AGU CCU ACU UAC UGA UGG AG | + | E7:17(8:12) |
| 10 | A7-6 | CCA AAG UCC UAC UUA CUG AU | − | E7:17(1:19) |
| 11 | A7-7 | AGA UAA CCA GGG GCA ACA GC | − | E7(20) |
| 12 | A7-8 | AGG AUA GAA GGC AAA GAC CU | − | E7(20) |
| 13 | A7-9 | GGC ACA UUA AAC UGA UGA AG | − | E7(20) |
| 14 | A7-10 | GGC CUC CAC CGG GGA UAU CG | + | E7(20) |
| 15 | A8-1 | CUG GAG AAC AAA GAA ACA AG | − | 17:E8(19:1) |
| 16 | A8-2 | AUC CCU ACA AAC UGG AGA AC | − | 17:E8(8:12) |
| 17 | A8-3 | GGC ACG GGA UCC CUA CAA AC | ++ | E8(20) |
| 18 | A8-4 | CUU CUC ACC UCU UUG ACA GG | ++ | E8:18(12:8) |
| 19 | A8-5 | UGG AGU CGU CCC UUC UCA CC | + | E8:18(1:19) |
| 20 | B7-1 | CUC CAA CAA UCA GAC CUA GG | +++ | 16:E7(5:15) |
| 21 | B7-2 | CAA UCA GAC CUA GGA AAA CG | + | 16:E7(11:9) |
| 22 | B7-3 | AGA CCU AGG AAA ACG GCA GG | − | 16:E7(16:4) |
| 23 | B7-4 | CCU UAC UUU UCC UCU GCA CC | − | E7:18(14:6) |
| 24 | B7-5 | GAG CAG AAC CUU ACU UUU CC | ++ | E7:18(6:14) |
| 25 | B7-6 | GAC GAG AGC AGA ACC UUA CU | ++ | E7:17(1:19) |
| 26 | B7-7 | UCA GCA GAC CCA GUG AUG UC | ++ | E7(20) |
| 27 | B7-8 | AUG AUG CAG UUC ACC AGU CC | + | E7(20) |
| 28 | B7-9 | UCA CCA GUC CUA ACA UCA GC | ++ | E7(20) |
| 29 | B7-10 | CCU CUG GAC CAG GAU GAU GC | ++ | E7(20) |
| 30 | B8-1 | UUC UCU ACA AUG AAG AGA GG | − | 17:E8(16:4) |
| 31 | B8-2 | GGC UUC UCU ACA AUG AAG AG | − | 17:E8(13:7) |
| 32 | B8-3 | UGU AGG CAG GAG GGC UUC UC | ++ | 17:E8(1:19) |
| 33 | B8-4 | ACU CAC CAC CUU GGC AUC UC | ++ | E8:18(14:6) |
| 34 | B8-5 | GCA GAG GGA UAC UCA CCA CC | − | E8:18(4:16) |

*SSOs with the prefix “A” are directed to TNFR1 and with “B” to TNFR2.

TABLE 2

LNA-2′deoxy-ribonucleosidephosphorothioate
chimeric mouse targetdd SSO

| | | | | |
|---|---|---|---|---|
| SEQ | | | | |
| ID. | Name | Sequence 5′ to 3′ | Activity | Target Site |

| | | | | |
|---|---|---|---|---|
| | | TNFR2 Exon 7 | | |
| 35 | 3272 | CAA TCA GAC CTA GGA A | − | 16:E7(7:9) |
| 36 | 3303 | CAA CAA TCA GAC CTA G | − | 16:E7(4:12) |
| 37 | 3304 | CAG ACC TAG GAA AAG G | − | 16:E7(11:5) |
| 38 | 3305 | AGC AGA CCC AGT GAT G | ++ | E7(16) |
| 39 | 3306 | CCA GTC CTA ACA TCA G | + | E7(16) |
| 40 | 3307 | CAC CAG TCC TAA CAT C | + | E7(16) |
| 41 | 3308 | CTG CAC CAG GAT GAT G | + | E7(16) |
| 42 | 3309 | ACT TTT CCT CTG GAC C | + | E7:17(14:2) |
| 43 | 3310 | CCT TAC TTT TCC TCT G | − | E7:17(8:8) |
| 44 | 3311 | CAG AAC CTT ACT TTT C | ++ | E7:17(5:11) |
| 45 | 3274 | AGA GCA GAA CCT TAC T | ++ | E7:17(1:15) |
| 46 | 3312 | GAG AGC AGA ACC TTA C | ++ | E7:17(0:16) |
| 47 | 3273 | ACC TTA TT TTC CTC T | − | E7:17(9:7) |
| | | TNFR2 Exon 8 | | |
| 48 | 3313 | CTT CTC TAC AAT GAA G | − | 17:E8(11:5) |
| 49 | 3314 | CCT TGG CAT CTC TTT G | − | E8(16) |
| 50 | 3315 | TCA CCA CCT TGG CAT C | + | E8:18(12:4) |
| 51 | 3316 | ACT CAC CAC CTT GGC A | + | E8:18(10:6) |
| 52 | 3317 | GAT ACT CAC CAC CTT G | + | E8:18(7:9) |
| 53 | 3631 | CTA CAA TGA AGA GAG G | − | 17(16) |
| 54 | 3632 | CTC TAC AAT GAA GAG A | − | 17:E8(14:2) |
| 55 | 3633 | AGG GAT ACT CAC CAC C | + | E8:18(4:12) |
| 56 | 3634 | CAG AGG GAT ACT CAC C | + | E8:18(1:15) |
| 57 | 3635 | CGC AGA GGG ATA CTC A | + | 18(16) |
| 58 | 3636 | GAA CAA GTC AGA GGC A | − | 17(16) |
| 59 | 3637 | GAG GCA GGA CTT CTT C | − | 17(16) |
| | | TNFR1 Exon 7 | | |
| 60 | 3325 | CGC AGT ACC TGC ACA C | + | 16:E7(8:8) |
| 61 | 3326 | AGT ACC TGC AGA CCA G | − | 16:E7(11:5) |
| 62 | 3327 | GGC AAC AGC ACC GCA G | − | E7(16) |
| 63 | 3328 | CTA GCA AGA TAA CCA G | − | E7(16) |
| 64 | 3329 | GCA CAT TAA ACT GAT G | − | E7(16) |
| 65 | 3330 | CTT CGG GCC TCC ACC G | − | E7(16) |
| 66 | 3331 | CTT ACT GAT GGA GTA G | − | E7:17(11:5) |
| 67 | 3332 | CCT ACT TAC TGA TGG A | − | E7:17(7:9) |
| 68 | 3333 | GTC CTA CTT ACT GAT G | −30 | E7:17(5:11) |
| | | TNFR1 Exon 8 | | |
| 69 | 3334 | TCC CTA CAA ACT GGA G | + | E7:17(5:11) |
| 70 | 3335 | GGC ACG GGA TCC CTA C | + | E8(16) |
| 71 | 3336 | CTC TTT GAC AGG CAC G | + | E8(16) |
| 72 | 3337 | CTC ACC TCT TTG ACA G | − | E8:18(11:5) |
| 73 | 3338 | CCT TCT CAC CTC TTT G | − | E8:18(7:9) |

TABLE 3

LNA-2′deoxy-ribonucleosidephosphorothioate
chimeric human targeted SSO

| | | | | |
|---|---|---|---|---|
| SEQ | | | | |
| ID. | Name | Sequence 5′ to 3′ | Activity | Target Site |

| | | | | |
|---|---|---|---|---|
| | | TNFR2 Exon 7 | | |
| 74 | 3378 | CCA CAA TCA GTC CTA G | ++ | 16:E7(4:12) |
| 75 | 3379 | CAG TCC TAG AAA GAA A | ++ | 16:E7(11:5) |
| 76 | 3380 | AGT AGA CCC AAG GCT G | − | E7(16) |
| 77 | 3381 | CCA CTC CTA TTA TTA G | + | E7(16) |
| 78 | 3382 | CAC CAC TCC TAT TAT T | + | E7(16) |
| 79 | 3383 | CTG GGT CAT GAT GAC A | − | E7(16) |
| 80 | 3384 | ACT TTT CAC CTG GGT C | ++ | E7:17(14:2) |
| 81 | 3385 | TCT TAC TTT TCA CCT G | − | E7:17(10:6) |
| 82 | 3459 | TGG ACT CTT ACT TTT C | ++ | E7:17(5:11) |
| 83 | 3460 | AGG ATG GAC TCT TAC T | − | E7:17(1:15) |
| 84 | 3461 | AAG GAT GGA CTC TTA C | + | 17(16) |
| | | TNFR2 Exon 8 | | |
| 85 | 3462 | CTT CTC TAT AAA GAG G | − | 17:E8(11:5) |
| 86 | 3463 | CCT TGG CTT CTC TCT G | + | E8(16) |
| 87 | 3464 | TCA CCA CCT TGG CTT C | + | E8:18(12:4) |
| 88 | 3465 | ACT CAC CAC CTT GGC T | + | E8:18(10:6) |
| 89 | 3466 | GAC ACT CAC CAC CTT G | + | E8:18(7:9) |
| | | TNFR1 Exon 7 | | |
| 90 | 3478 | TGT GGT GGC TGC AGA C | N/A | 16:E7(8:8) |
| 91 | 3479 | GGT GCC TGC AGA CAA A | N/A | 16:E7(11:5) |
| 92 | 3480 | GGC AAC AGC ACT GTG G | N/A | E7(16) |
| 93 | 3481 | CAA AGA AAA TGA CCA G | N/A | E7(16) |
| 94 | 3482 | ATA CAT TAA ACC AAT G | N/A | E7(16) |
| 95 | 3483 | GCT TGG ACT TCC ACC G | N/A | E7(16) |
| 96 | 3484 | CTC ACC AAT GGA GTA G | N/A | E7:17(11:5) |
| 97 | 3485 | CAC TCA CCA ATG GAG T | N/A | E7:17(9:7) |
| 98 | 3587 | CCC ACT CAC CAA TGG A | N/A | E7:17(7:9) |
| 99 | 3588 | CCC CCA CTC ACC AAT G | N/A | E7:17(5:11) |
| 100 | 3589 | AAA GCC CCC ACT CAC C | N/A | E7:17(1:15) |
| | | TNFR1 Exon 8 | | |
| 101 | 3590 | TTT CCC ACA AAC TGA G | N/A | 17:E8(5:11) |
| 102 | 3591 | GGT GTC GAT TTC CCA C | N/A | E8(16) |
| 103 | 3592 | CTC TTT TTC AGG TGT C | N/A | E8(16) |
| 104 | 3593 | CTC ACC TCT TTT TCA G | N/A | E8:18(11:5) |
| 105 | 3594 | TCA TCT CAC CTC TTT T | N/A | E8:18(7:9) |
| | | Control | | |
| 106 | 3083 | GCT ATT ACC TTA ACC C | N/A | N/A |

### Example 4

### Effect of SSOs on L929 Mouse Cells

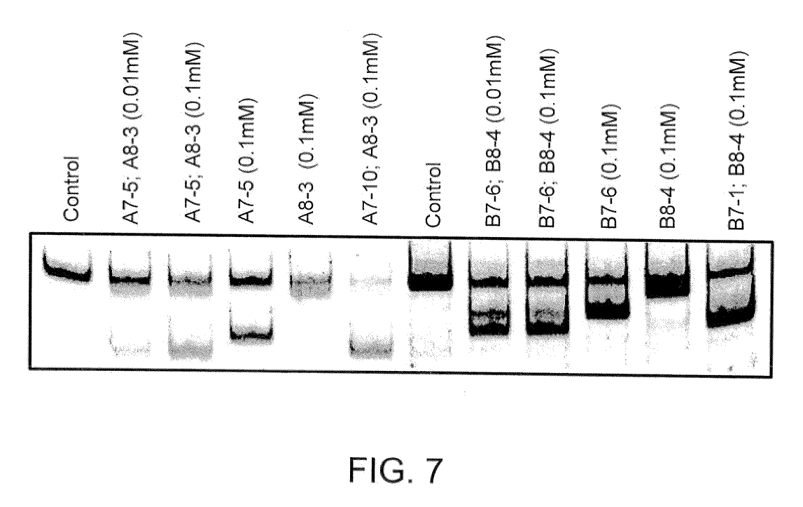

Single LNA SSOs were transfected into L929 murine cells and analyzed for splice switching of TNFR2. FIG. 9 (top) shows the splice switching results of LNAs targeted towards mouse exon 7. Of the LNAs tested, at least 9 showed some activity. In particular, LNA 3312, 3274 and 3305 induced skipping of exon 7 to 50% or greater; LNA 3305 treatment resulted in almost complete skipping. FIG. 9 (bottom) shows the activity of SSOs targeted towards mouse exon 8. The data indicate that LNA 3315 and 3316 are equally potent at inducing an approximately 20% skipping of exon 8. Note that exon 8 is small (35 nts), and therefore the difference in exon 8-containing and exon 8-lacking PCR fragments is also small.

### Example 5

### Effect of Multiple SSOs on L929 Mouse Cells

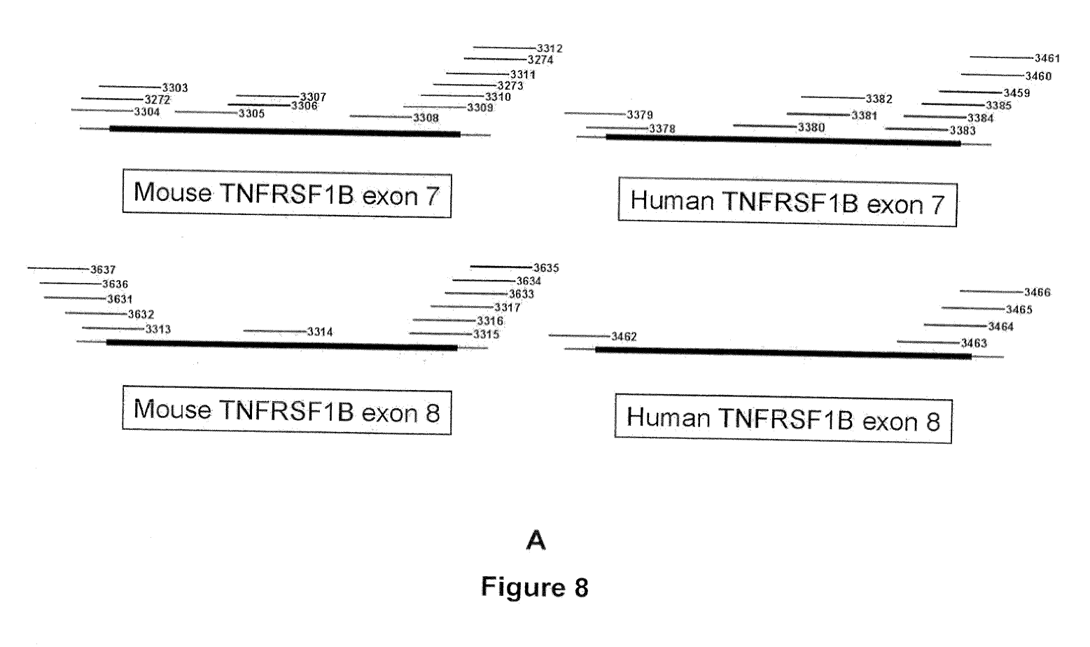

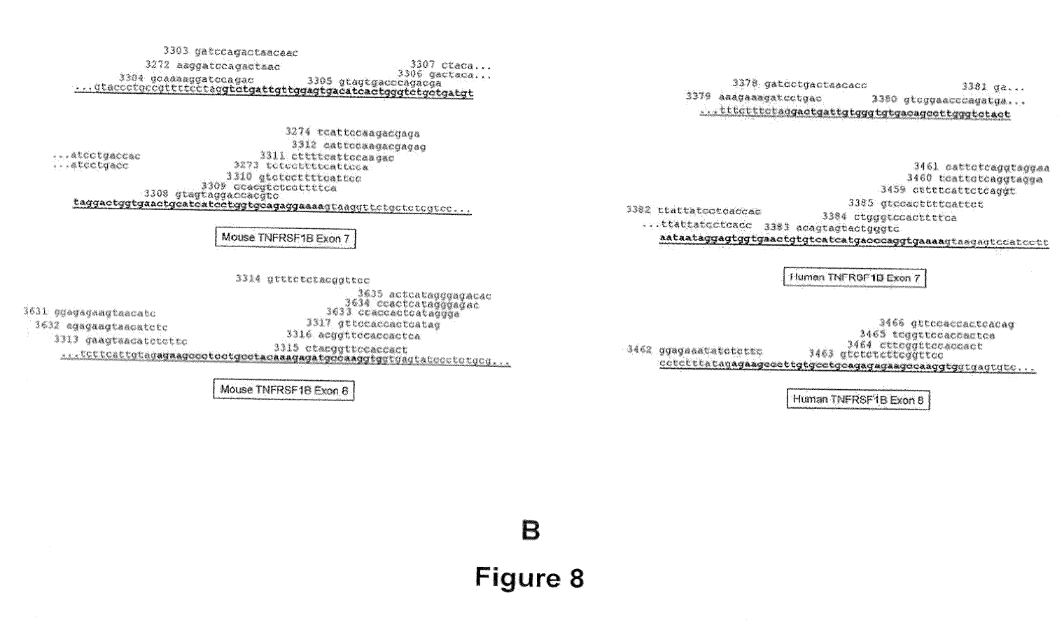

LNA SSOs targeting exon 7 and 8 were transfected in combination into L929 cells to determine whether such treatment would result in generation of TNFR2 Δ7/8 mRNA. The data in FIG. 10 show that the combination of exon 8 targeted 3315 or 3316 with one of exon 7 targeted LNA 3305, 3309, 3312, or 3274 induced skipping of both exons simultaneously. In particular, the combination of LNAs 3305 and 3315 resulted in greater than 60% shift to the Δ7/8 mRNA, with the remainder being almost entirely Δ7 mRNA. Other combinations were also effective; 3274 with 3315 led to a 50% shift to the Δ7/8 mRNA. These data indicate that LNA SSOs are very effective at inducing alternatively spliced TNFR2 mRNAs. Similarly, combinations of LNA SSOs targeted to TNFR1 exon 7 and 8 also induced shifting of their respective exons in L929 cells (FIG. 11).

### Example 6

### Effect of LNA SSOs on Primary Mouse Hepatocytes

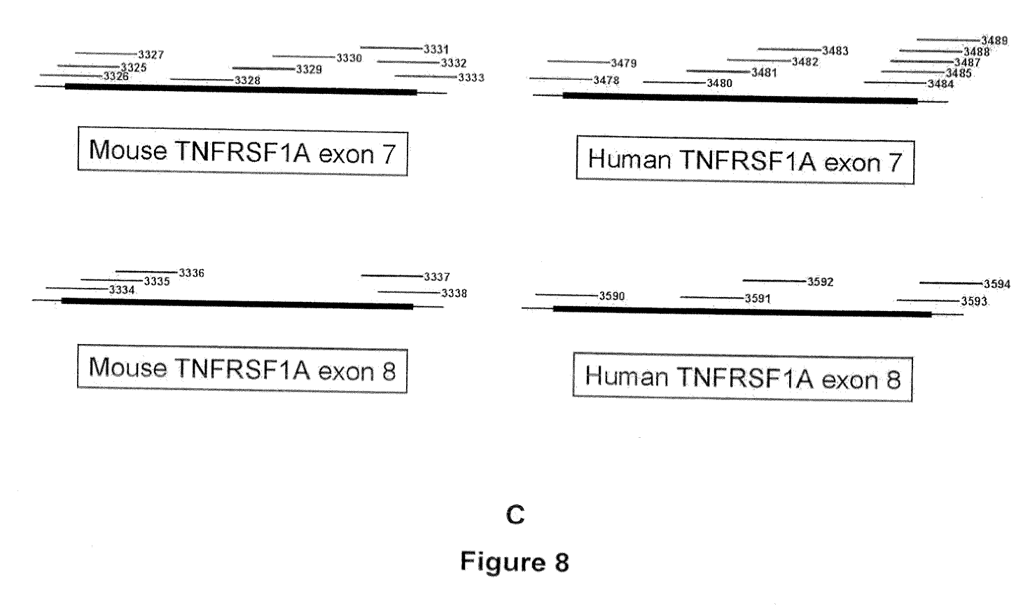

The TNFR2 LNA SSOs were transfected into primary mouse hepatocytes, and were found to be equally effective in splice switching in these cells. In particular, treatment with LNA 3274 or 3305 in combination with LNA 3315 showed splice shifting profiles very similar to those found in L929 cells (FIG. 12). These data confirm splice shifting occurs in intended in vivo cellular targets.

### Example 7

### Secretion of TNFR2 Splice Variants from Murine Cells

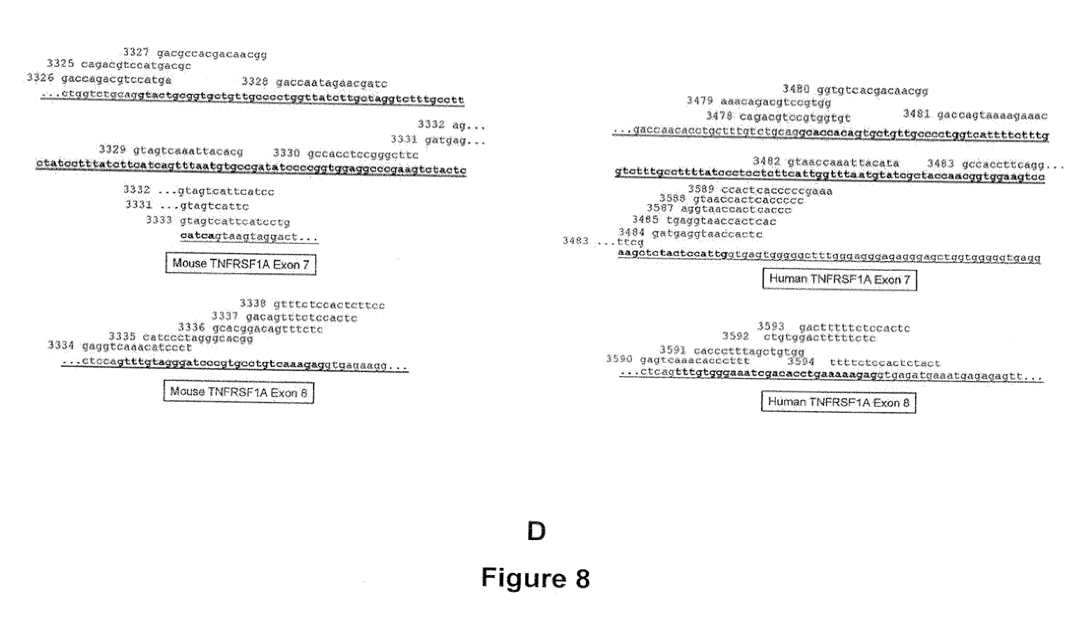

The ability of LNA SSOs to induce soluble TNFR2 protein production and secretion into the extracellular media was tested. L929 cells were treated with the LNA SSOs as above, and extracellular media samples were collected 48 hours after transfection. The samples were quantified by an ELISA specific for soluble TNFR2 (for either Δ7 and Δ7/8 protein isoforms). The FIG. 13 left panel indicates that the LNAs that best induced shifts in RNA splicing, also secreted the most protein into the extracellular media. In particular, LNAs 3305, 3312 and 3274 performed best, increasing soluble TNFR2 at least 3.5-fold over background, and yielding 250 pg/mL soluble splice variant. Increases were also seen in similarly treated primary mouse hepatocytes (FIG. 13, right panel). In these primary cells, treatment with LNA 3274 or 3305 alone gave approximately 2.5-fold increases in soluble TNFR2 in the extracellular media, yielding ˜200 pg/mL of the soluble splice variant, and the combination of 3274 or 3305 with 3315 also increased protein production. Consequently, induction of the splice variant mRNA correlated with production and secretion of the soluble TNFR2.

### Example 8

### Effect of LNA SSOs on Primary Human Hepatocytes

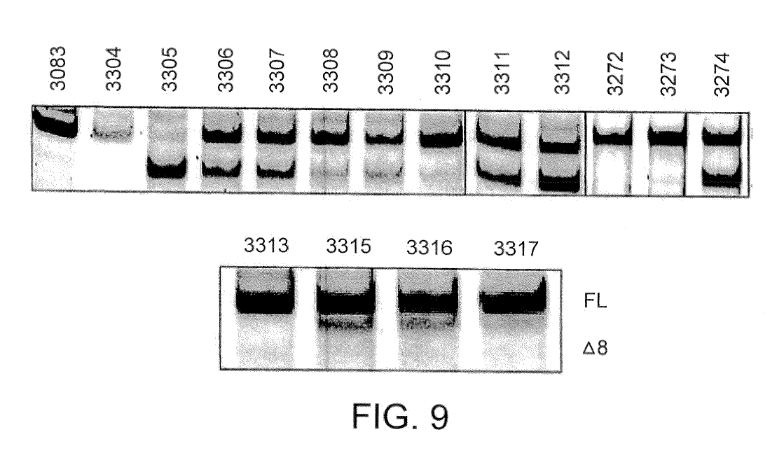

LNA SSOs for human TNFR2 pre-mRNA were transfected into cultured primary human hepatocytes. FIG. 14 shows that 7 of 10 SSOs targeted to exon 7 exhibited some splice switching activity. In particular, LNAs 3378, 3384 and 3479 showed at least 75% skipping of exon 7. Likewise, 4 of the 5 exon 8 targeted SSOs showed activity. Interestingly, LNAs 3464, 3465, or 3466 alone was sufficient to induce Δ7/8 splice removal, an observation not seen in mouse cells. Hence, only one SSO may be required to induce skipping of both exon 7 and exon 8. These data confirm splice shifting occurs in intended human therapeutic targets.

### Example 9

### In Vivo Injection of LNA SSOs in Mice

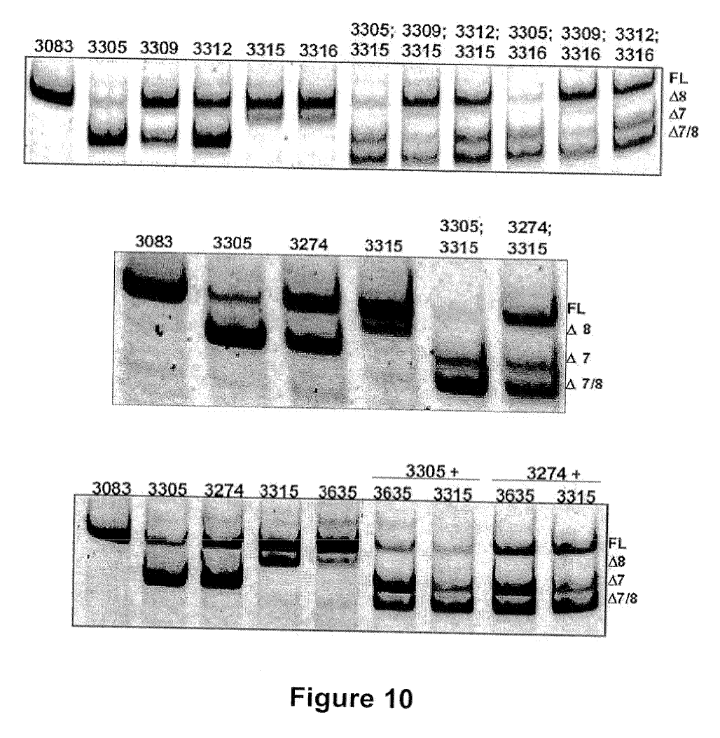

LNA 3305, at doses from 3 mg/kg to 25 mg/kg diluted in saline only, were injected intraperitoneal (i.p.) once a day for 4 days into mice. The mice were sacrificed on day 5 and total RNA from the liver was analyzed by RT-PCR. The data show splice switching efficacy similar to that found in cell culture. At the maximum dose of 25 mg/kg, LNA 3305 induced almost full conversion to Δ7 mRNA (FIG. 15, bottom panel).

A similar procedure using LNA 3274 induced about 20% conversion to Δ7 mRNA. To optimize the induction of Δ7 mRNA LNA 3274, both the dose regimen and time between the last injection, and sacrifice of the animals was varied. LNA 3274, at 25 mg/kg diluted in saline only, were injected (i.p.) once a day for 4 days into mice. In mice analyzed on day 15, whereas those analyzed on day five demonstrated only a 20% shift to Δ7 mRNA (FIG. 15, top panel). Furthermore, mice given injections for 10 (lays, and sacrificed on day 11 showed a 50% induction of Δ7 mRNA (FIG. 15 top). These in vivo data suggest that TNFR2 LNA SSOs can persist in the liver and induce splice switching for at least 10 days after administration.

### Example 9

### Circulatory TNFR Splice Variants

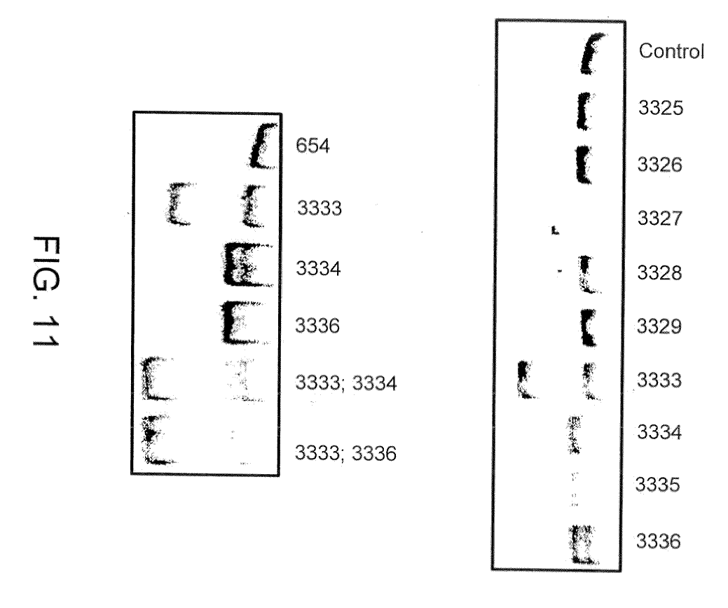

Induction of the Δ7 in RNA in liver should produce soluble TNFR, which can be secreted and accumulate in the circulation. Accordingly, mice were treated with LNA 3274, 3305, or the control 3083 alone i.p. at 25 mg/kg/day for 10 days. Mice were bled before injection and again 1, 5 and 10 days after the last injection. Serum was quantified for concentration of soluble TNFR2. FIG. 16 shows that LNA treatment induced 6000-8000 pg/ml of soluble TNFR2 (Δ7), which was significantly over background for at least 10 days.

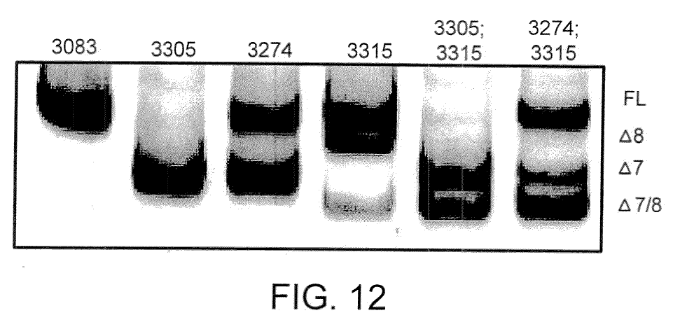

The same samples were assayed for production of soluble TNFR1. No increase in soluble TNFR1 was observed (FIG. 17).

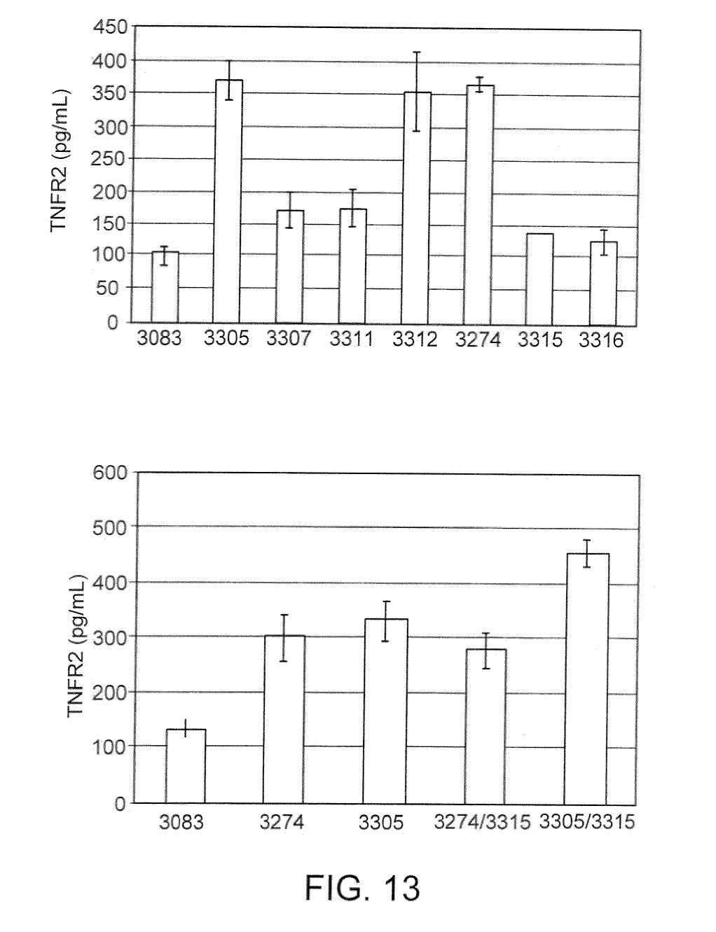

To test the effects at longer time points, the same experiment was carried out, and mice were analyzed for soluble TNFR2 in the serum up to 27 days after the last injection. The results show only a slight decrease in soluble TNFR2 levels 27 days after the last LNA SSO injection (FIG. 18). This data suggests that the effects of the LNAs persist for at least 27 days.

### Example 10

### Measurement of Anti-TNF-α Activity of Mice Treated with LNA SSOs

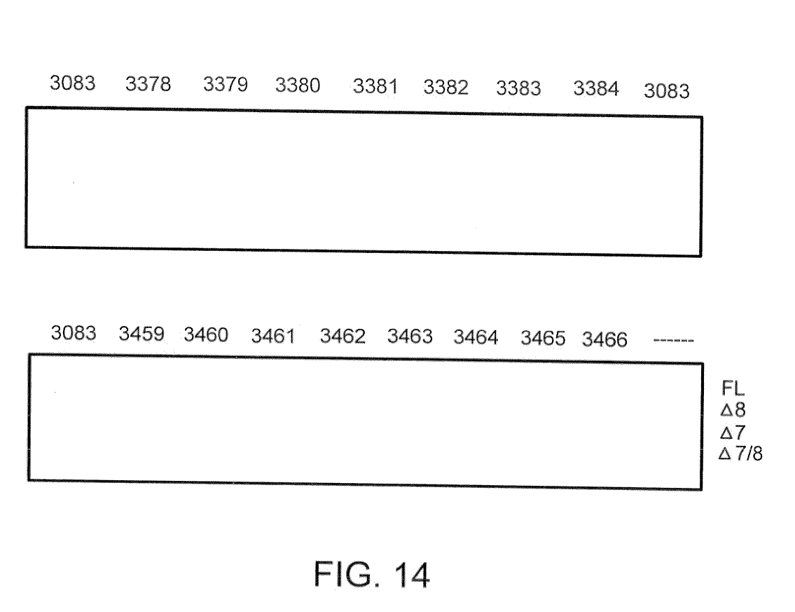

The anti-TNF-α activity of serum from LNA 3274 treated mice was tested in an L929 cytotoxicity assay. In this assay, serum is tested for its ability to protect cultured L929 cells from the cytotoxic effects of a fixed concentration of TNF-α. L929 cells were seeded in 96-well plates at 2×10⁴ cells per well in 100 μL of complete MEM media (containing 10% regular FBS) and allowed to grow for 24 hours at 37° C. As shown in FIG. 19, serum from mice treated with LNA 3274 but not control LNAs (3083 or 3272) increased viability of the L929 cells exposed to 0.1 ng/mL TNF-α. Hence, the LNA 3274 serum contained Δ7 TNFR2 TNF-α antagonist, sufficient to bind and inactivate TNF-α, and thereby protect the cells from the cytotoxic effects of TNF-α. This anti-TNF-α activity was present in the serum of animals 5 and 27 days after the last injection of the 3274 LNA.

### Example 11

### Comparison of LNA SSOs to Other Anti-TNF-α Agents

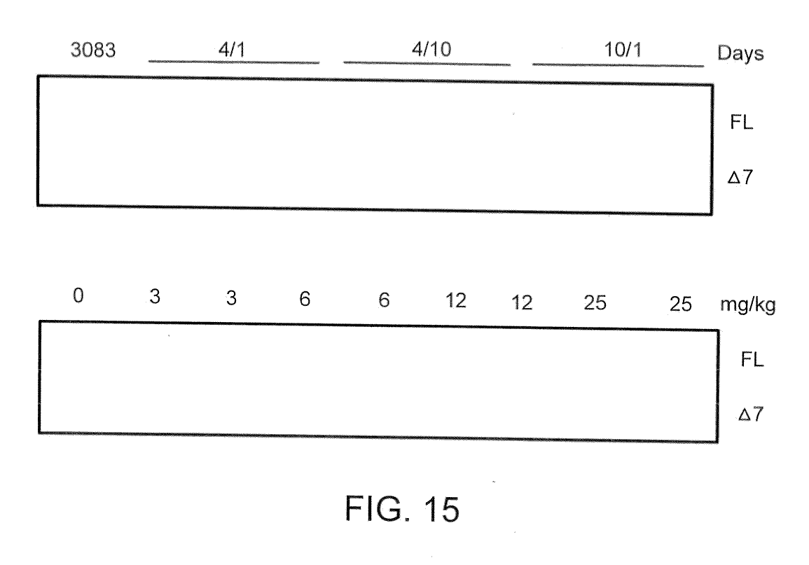

L929 cells were seeded as described previously. Samples were prepared containing 90 μL of serum-free MEM, 0.1 ng/ml TNF-α (TNF) and 1 μg/ml of actinomycin D (ActD), with either (i) rsTNFR2 (recombinant soluble) (0.01-3 μg/mL), (ii) serum from LNA 3274 treated mice (1.25-10%, diluted in serum from untreated mice) or (iii) Enbrel® (0.45-150 pg/ml) to a final volume of 100 μl with a final mouse serum concentration of 10%. The samples were incubated at room temperature for 30 minutes. Subsequently, the samples were applied to the plated cells and incubated for ˜24 hours at 37° C. in a 5% CO₂ humidified atmosphere. Cell viability was measured by adding 20 μL CellTiter 96® Aqueous Solution (Promega) and measuring absorbance at 490 nm with a microplate reader. Cell viability, as shown in FIG. 20, was normalized to cells untreated with TNF/ActD.

### Example 12

Oligonucleotides. Table 6 lists chimeric locked nucleic acid (LNA) SSOs with alternating 2′deoxy- and 2′O-4′-(methylene)-bicyclic-ribonucleoside phosphorothioates and having sequences as described as above. These were synthesized by Santaris Pharma, Denmark. For each SSO, the 5′-terminal nucleoside was a 2′O-4′-methylene-ribonucleoside and the 3′-terminal nucleoside was a 2′deoxy-ribonucleoside. Table 7 shows the sequences of chimeric LNA SSOs with alternating 2′-O-methyl-ribonucleoside-phosphorothioates (2′-OMe) and 2′O-4′-(methylene)-bicyclic-ribonucleoside phosphorothioates. These were synthesized by Santaris Pharma, Denmark. The LNA is shown in capital letters and the 2′-OME is shown in lower case letters.

Cell culture and transfections. L929 cells were maintained in minimal essential media supplemented with 10% fetal bovine serum and antibiotic (37° C., 5% CO₂). For transfection, L929 cells were seeded in 24-well plates at 10⁵ cells per well and transfected 24 hrs later. Oligonucleotides were complexed, at the indicated concentrations, with 2 μL of Lipofectamine™ 2000 transfection reagent (Invitrogen) as per the manufacturer's directions. The nucleotide/lipid complexes were then applied to the cells and incubated for 24 his. The media was then aspirated and cells harvested with TRI-Reagent™ (MRC, Cincinnati, Ohio).

RT-PCR. Total RNA was isolated with TRI-Reagent (MRC, Cincinnati, Ohio) and TNFR1 or TNFR2 mRNA was amplified by GeneAmp® RT-PCR using rTth polymerase (Applied Biosystems) following supplier directions. Approximately 200 ng of RNA was used per reaction. Primers used in the examples described herein are included in Table 2. Cycles of PCR proceeded: 95° C., 60 sec; 56° C., 30 sec; 72° C., 60 sec for 22-30 cycles total.

In some instances a Cy5-labeled dCTP (GE Healthcare) was included in the PCR step for visualization (0.1 μL per 50 μL PCR reaction). The PCR products were separated on a 10% non-denaturing polyacrylamide gel, and Cy5-labeled bands were visualized with a Typhoon™ 9400 Scanner (GE Healthcare). Scans were quantified with ImageQuant™ (GE Healthcare) software. Alternatively, in the absence of the inclusion of Cy5-labeled dCTP, the PCR products were separated on a 1.5% agarose gel containing trace amounts of ethidium bromide for visualization.

PCR. PCR was performed with Platinum® Taq DNA Polymerase (Invitrogen) according to the manufacturer's directions. For each 50 μL reaction, approximately 30 pmol of both forward and reverse primers were used. Primers used in the examples described herein are included in Table 5. Thermocycling reaction proceeded, unless otherwise stated, as follows: 94° C., 3 minutes; then 30-40 cycles of 94° C., 30 sec; 55° C., 30 sec; and 72° C., 105 sec; followed by 72° C., 3 minutes. The PCR products were analyzed on 1.5% agarose gels and visualized with ethidium bromide.

TABLE 5
RT-PCR and PCR Primers

| SEQ | | |
|---|---|---|
| ID. | Name | Sequence 5′ to 3′ |
| | | Human TNFR2 |
| 190 | TR001 | ACT GGG CTT CAT CCC AGC ATC |
| 191 | TR002 | CAC CAT GGC GCC CGT CGC CGT CTG G |
| 192 | TR003 | CGA CTT CGC TCT TCC AGT TGA GAA GCC CTT |
| | | GTG CCT GCA G |
| 193 | TR004 | TTA ACT GGG CTT CAT CCC AGC ATC |
| 194 | TR005 | CTG CAG GCA CAA GGG CTT CTC AAC TGG AAG |
| | | AGC GAA GTC G |
| 195 | TR026 | TTA ACT GGG CTT CAT CCC AGC |
| 196 | TR027 | CGA TAG AAT TCA TGG CGC CCG TCG CCG TCT |
| | | GG |
| 197 | TR028 | CCT AAC TCG AGT TAA CTG GGC TTC ATC CCA |
| | | GC |
| 198 | TR029 | GAC TGA GCG GCC GCC ACC ATG GCG CCC GTC |
| | | GCC GTC TGG |
| 199 | TR030 | CTA AGC GCG GCC GCT TAA CTG GGC TTC ATC |
| | | CCA GCA TC |
| 200 | TR047 | CGT TCT CCA ACA CGA CTT CA |
| 201 | TR048 | CTT ATC GGC AGG CAA GTG AGG |
| 202 | TR049 | ACT GAA ACA TCA GAC CTG GTG TGC |
| 203 | TR050 | CCT TAT CGG CAG GCA ACT GAG |
| | | Human TNFR1 |
| 204 | TR006 | CCT CAT CTG AGA AGA CTG GGC G |
| 205 | TR007 | GCC ACC ATG GGC CTC TCC ACC GTG C |
| 206 | TR008 | GGG CAC TGA GGA CTC AGT TTG TGG GAA ATC |
| | | GAC ACC TG |
| 207 | TR009 | CAG GTG TCG ATT TCC CAC AAA CTG AGT CCT |
| | | CAG TGC CC |
| 208 | TR010 | CAC CAT GGG CCT CTC CAC CGT GC |
| 209 | TR011 | TCT GAG AAG ACT GGG CG |
| 210 | TR031 | CGA TAG GAT CCA TGG GCC TCT CCA CCG TGC |
| 211 | TR032 | CCT AAC TCG AGT CAT CTG AGA AGA CTG GGC G |
| 212 | TR033 | GAC TGA GCG GCC GCC ACC ATG GGC CTC TCC |
| | | ACC GTG C |
| 213 | TR034 | CTA AGC GCG GCC GCT CAT CTG AGA AGA CTG |
| | | GGC G |
| | | Mouse TNFR2 |
| 214 | TR012 | GGT CAG GCC ACT TTG ACT GC |
| 215 | TR013 | CAC CGC TGC CCC TAT CGC G |
| 216 | TR014 | CAC CGC TGC CAC TAT GGC G |
| 217 | TR015 | GGT CAG GCC ACT TTG ACT GCA ATC |
| 218 | TR016 | GCC ACC ATG GCG CCC GCC GCC CTC TGG |
| 219 | TR017 | GGC ATC TCT CTT CCA ATT GAG AAG CCC TCC |
| | | TGC CTA CAA CC |
| 220 | TR018 | CTT TGT AGG CAG GAG GGC TTC TCA ATT GGA |
| | | AGA GAG ATG CC |
| 221 | TR019 | GGC CAC TTT GAC TGC AAT CTG |
| 222 | TR035 | CAC CAT GGC GCC CGC CGC CCT CTG G |
| 223 | TR036 | TCA GGC CAC TTT GAC TGC AAT C |
| 224 | TR037 | CGA TAG AAT TCA TCG CGC CCG CCG CCC TCT |
| | | GG |
| 225 | TR038 | CCT AAC TCG AGT CAG GCC ACT TTG ACT GCA |
| | | ATC |
| 226 | TR039 | GAC TGA GCG GCC GCC ACC ATG GCG CCC GCC |
| | | GCC CTC TGG |
| 227 | TR040 | CTA AGC GCG GCC GCT CAG GCC ACT TTG ACT |
| | | GCA ATC |
| 228 | TR045 | GAG CCC CAA ATG GAA ATG TGC |
| 229 | TR046 | GCT CAA GGC CTA CTG CAT CC |
| | | Mouse TNFR1 |
| 230 | TR020 | GGT TAT CGC GGG AGG CGG GTC G |
| 231 | TR021 | GCC ACC ATG GGT CTC CCC ACC GTG CC |
| 232 | TR022 | CAC AAA CCC CCA GGA CTC AGT TTG TAG GGA |
| | | TCC CGT GCC T |
| 233 | TR023 | AGG CAC GGG ATC CCT ACA AAC TGA GTC CTG |
| | | GGG GTT TGT G |
| 234 | TR024 | CAC CAT GGG TCT CCC CAC CGT GCC |
| 235 | TR025 | TCG CGG GAG GCG GGT CGT GG |
| 236 | TR041 | CGA TAG TCG ACA TGG GTC TCC CCA CGG TGC C |
| 237 | TR042 | CCT AAG AAT TCT TAT CGC GGG AGG CGG GTC G |
| 238 | TR043 | GAC TGA GCG GCC GCC ACC ATG GGT CTC CCC |
| | | ACC GTG CC |
| 239 | TR044 | CTA AGC GCG GCC GCT TAT CGC GGG AGG CGG |
| | | GTC G |

Human hepatocyte cultures. Human hepatocytes were obtained in suspension either from ADMET technologies, or from The UNC Cellular Metabolism and Transport Core at UNC-Chapel Hill. Cells were washed and suspended in RPMI 1640 supplemented with 10% FBS, 1 mg/mL, human insulin, and 13 nM DexamethASONe. Hepatocytes were plated in 6-well plates at 0.5×10⁶ cells per plate in 3 mL media. After 1-1.5 hrs, non-adherent cells were removed, and the media was replaced with RPMI 1640 without FBS, supplemented with 1 mg/mL human insulin, and 130 nM DexamethASONe.

For delivery of SSOs to hepatocytes in 6-well plates, 10 mL of a 5 mM SSO stock was diluted into 100 mL of OPTI-MEM™, and 4 mL of Lipofectamine™ 2000 was diluted into 100 mL of OPTI-MEM™. The 200 mL complex solution was then applied to the cells in the 6-well plate containing 2800 mL of media, for a total of 3000 mL. The final SSO concentration was 17 nM. After 24 hrs, cells were harvested in TRI-Reagent™. Total RNA was isolated per the manufacturer's directions. Approximately 200 ng of total RNA was subjected to reverse transcription-PCR (RT-PCR).

ELISA. To determine the levels of soluble TNFR2 in cell culture media or sera, the Quantikine® Mouse sTNF RII ELISA kit from R&D Systems (Minneapolis, Minn.) or Quantikine® Human sTNF RII ELISA kit from R&D Systems (Minneapolis, Minn.) were used. The antibodies used for detection also detect the protease cleavage forms of the receptor. ELISA plates were read using a microplate reader set at 450 nm, with wavelength correction set at 570 nm.

For mouse in vivo studies, blood from the animals was clotted for 1 hour at 37° C. and centrifuged for 10 min at 14,000 rpm (Jouan BRA4i centrifuge) at 4° C. Sera was collected and assayed according to the manufacturer's guide, using 50 mL of mouse sera diluted 1:10.

L929 cytotoxicity assay. L929 cells plated in 96-well plates at 10⁴ cells per well were treated with 0.1 ng/mL TNF-α and 1 mg/mL actinomycin D in the presence of 10% serum from mice treated with the indicated oligonucleotide in 100 mL total of complete MEM media (containing 10% regular FBS) and allowed to grow for ˜24 hrs at 37° C. Control lanes were plated in 10% serum from untreated mice. Cell viability was measured 24 hrs later by adding 20 mL CellTiter 96® AQᵤₑₒᵤₛ One Solution Reagent (Promega) and measuring absorbance at 490 nm with a microplate reader. Cell viability was normalized to untreated cells.

Western blots. Twenty mL of media or 20 mg of lysate were loaded in each well of a 4-12% NuPAGE® polyacrylamide gel (Invitrogen). The gel was run 40 min at 200V. The protein was transferred, for 1 hr at 30V, to an Invitrolon™ PVDF membrane (Invitrogen), which was then blocked with StartingBlock® Blocking Buffer (Pierce) for 1 hr at room temperature. The membrane was incubated for 3 hrs at room temperature with a rabbit polyclonal antibody that recognizes the C-terminus of human and mouse TNFR2 (Abeam), Following three washes in PBS-T buffer (1×PBS, 0.1% Tween-20), the membrane was incubated for one hour at room temperature with secondary goat anti-rabbit antibody (Abeam) and again washed three times with PBS-T buffer. The protein was then detected with ECL Plus™ (GE Healthcare), according to the manufacturer's recommendations and then photographed.

### Example 13

### SSO Splice Switching Activity with TNFR mRNA

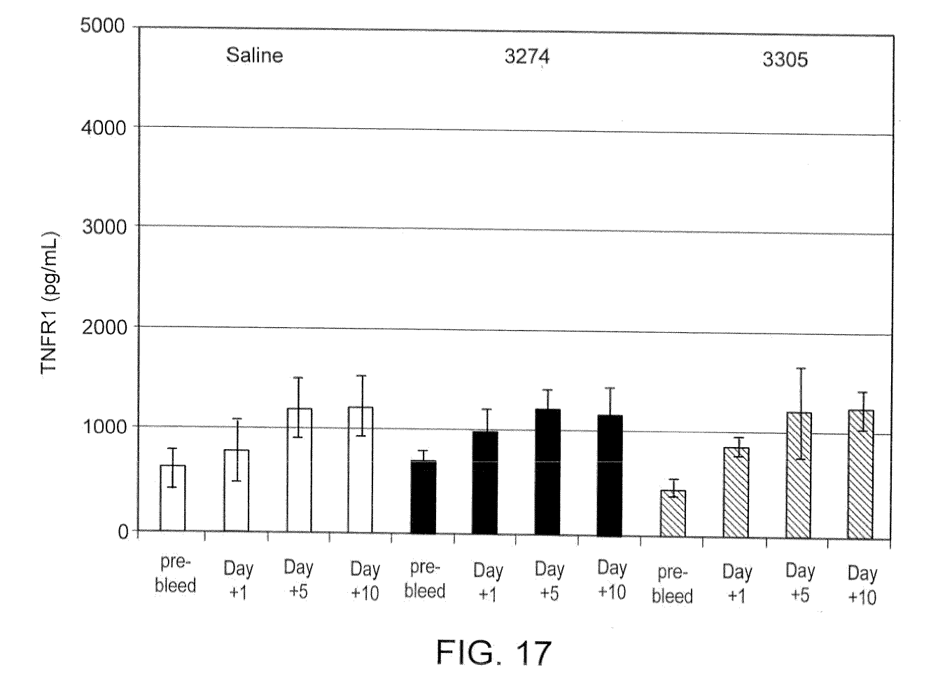

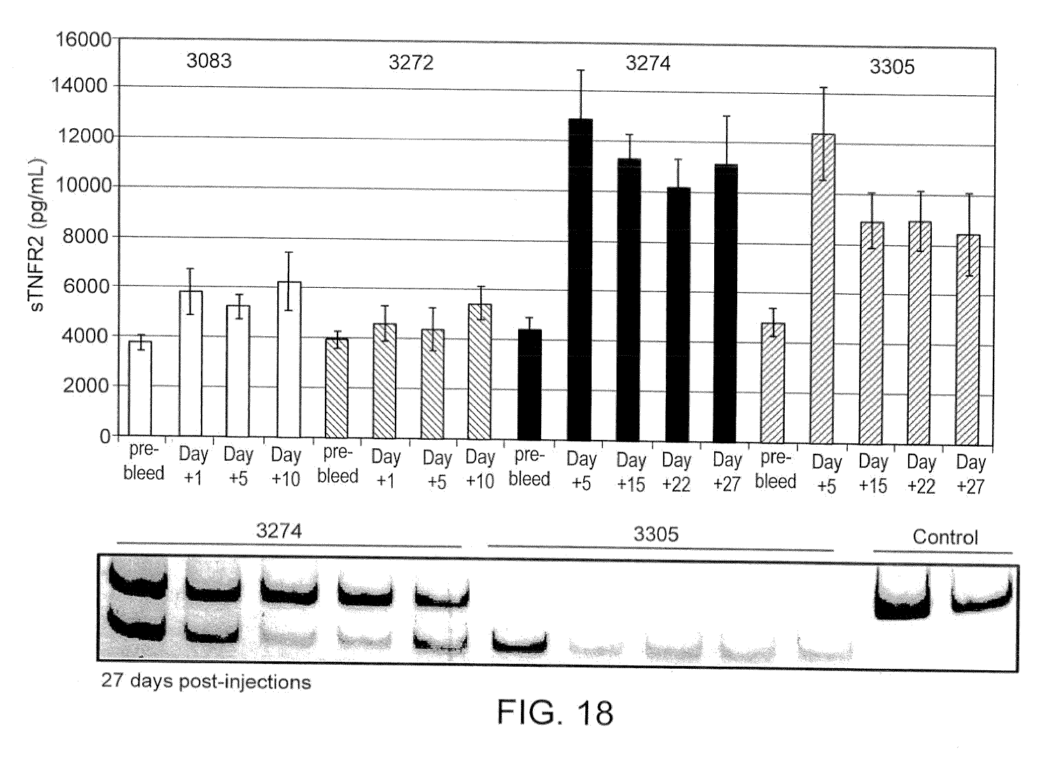

Table 6 shows the splice switching activities of SSOs having sequences as described in U.S. application Ser. No. 11/595,485 and targeted to mouse and human TNFRs. Of SSOs targeted to mouse TNFR2 exon 7, at least 8 generated some muTNFR2 Δ7 mRNA. In particular, SSO 3312, 3274 and 3305 induced at least 50% skipping of exon 7; SSO 3305 treatment resulted in almost complete skipping. Of SSOs transfected into primary human hepatocytes, and targeted to human TNFR2 exon 7, at least 7 SSOs generated some huTNFR2 Δ7 mRNA. In particular, SSOs 3378, 3379, 3384 and 3459 induced at least 75% skipping of exon 7 (FIG. 22B), and significant induction of huTNFR2 Δ7 into the extracellular media (FIG. 22A).

TABLE 6
SSO Splice Switching Activity

| | | |
|---|---|---|
| SEQ ID. | Name | Activity |

Mouse TNFR2

| | | |
|---|---|---|
| | 3272 | − |
| | 3304 | − |
| | 3305 | + |
| | 3306 | + |
| | 3307 | + |
| | 3308 | + |
| | 3309 | + |
| | 3310 | − |
| | 3311 | + |
| 62 | 3274 | + |
| | 3312 | + |
| | 3273 | − |

Mouse TNFR1

| | |
|---|---|
| 3333 | + |

Human TNFR2

| | | |
|---|---|---|
| 14 | 3378 | + |
| 30 | 3379 | + |
| | 3380 | − |
| 70 | 3381 | + |
| 71 | 3382 | + |
| | 3383 | − |
| 46 | 3384 | + |
| 72 | 3459 | + |
| | 3460 | − |
| 73 | 3461 | + |

Control

| | |
|---|---|
| 3083 | − |

Table 7 contains the sequences of 10 nucleotide chimeric SSOs with alternating 2′-O-methyl-ribonucleoside-phosphorothioates (2′-OMe) and 2′O-4′-(methylene)-bicyclic-ribonucleoside phosphorothioates. These SSOs are targeted to exon 7 of mouse TNFR2.

TABLE 7

LNA/2′-OMe-ribonucleosidephosphorothioate
chimeric mouse targeted SSO

| | | |
|---|---|---|
| SEQ ID. | Name | Sequence 5′ to 3′* |

| | | |
|---|---|---|
| 178 | 3274 | AgAgCaGaAcCtTaCt |
| 179 | 3837 | gAaCcTuAcT |
| 180 | 3838 | aGaGcAgAaC |
| 181 | 3839 | gAgCaGaAcC |
| 182 | 3840 | aGcAgAaCcT |
| 183 | 3841 | gCaGaAcCuT |
| 184 | 3842 | cAgAaCcTuA |
| 185 | 3843 | aGaAcCuTaC |

*Capital letters are 2′O-4′-(methylene)-bicyclic-ribonucleosides; lowercase letters are 2′-OMe

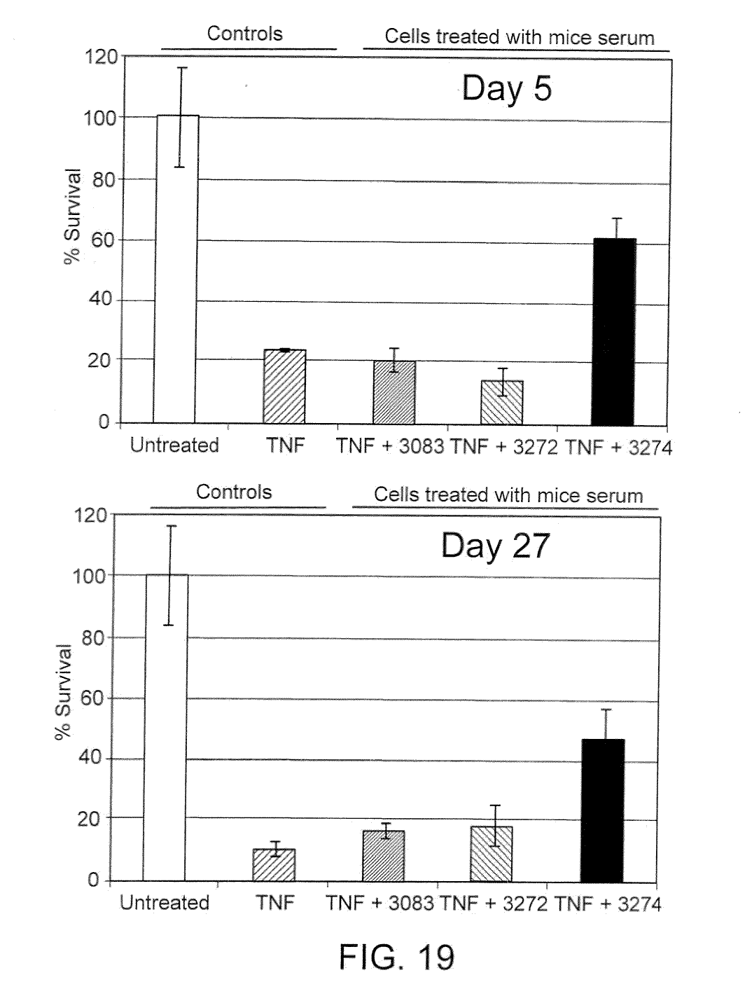

To analyze the in vitro splice-switching activity of the SSOs listed in Table 7, L929 cells were cultured and seeded as described in Example 12. For delivery of each of the SSOs in Table 7 to the L929 cells, SSOs were diluted into 50 mL of OPTI-MEM™, and then 50 mL, Lipofectamine™ 2000 mix (1 part Lipofectamine™ 2000 to 25 parts OPTI-MEM™) was added and incubated for 20 minutes. Then 400 mL of serum free media was added to the SSOs and applied to the cells in the 24-well plates. The final SSO concentration was either 50 or 100 nM. After 24 hrs, cells were harvested in 800 mL TRI-Reagent™. Total RNA was isolated per the manufacturer's directions and analyzed by RT-PCR (FIG. 23) using the forward primer TR045 (SEQ ID No: 228) and the reverse primer TR046 (SEQ ID No: 229).

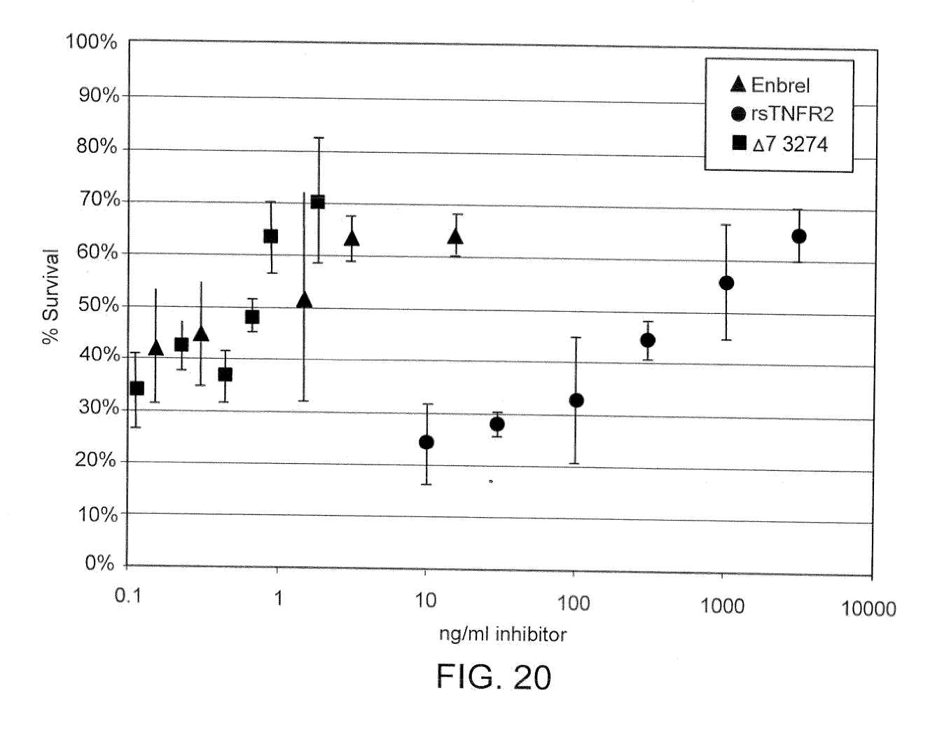

To analyze the in vivo splice-switching activity of the SSOs listed in Table 7, mice were injected with the SSOs listed in Table 4 intraperitoneal (i.p.) at 25 mg/kg/day for 5 days. Mice were bled before injection and again 1, 5 and 10 days after the last injection. The concentration of soluble TNFR2 Δ7 in the sera taken before the first injection and 10 days after the last injection were measured by ELISA (FIG. 24B). The mice were sacrificed on day 10 and total RNA from 5-10 mg of the liver was analyzed by RT-PCR (FIG. 24A) using the forward primer TR045 (SEQ ID No: 228) and the reverse primer TR046 (SEQ ID No: 229).

Of the 10 nucleotide SSOs subsequences of SSO 3274 tested in vitro, all of them generated at least some muTNFR2 Δ7 mRNA (FIG. 23). In particular, SSO 3839, 3840 and 3841 displayed greater splice-switching activity than the longer 16 nucleotide SSO 3274 from which they are derived. The three 10 nucleotide SSOs, 3839, 3840, 3841, that demonstrated the greatest activity in vitro also were able to generate significant amounts of muTNFR2 Δ7 mRNA (FIG. 24A) and soluble muTNFR2 Δ7 protein (FIG. 24B) in mice in vivo.

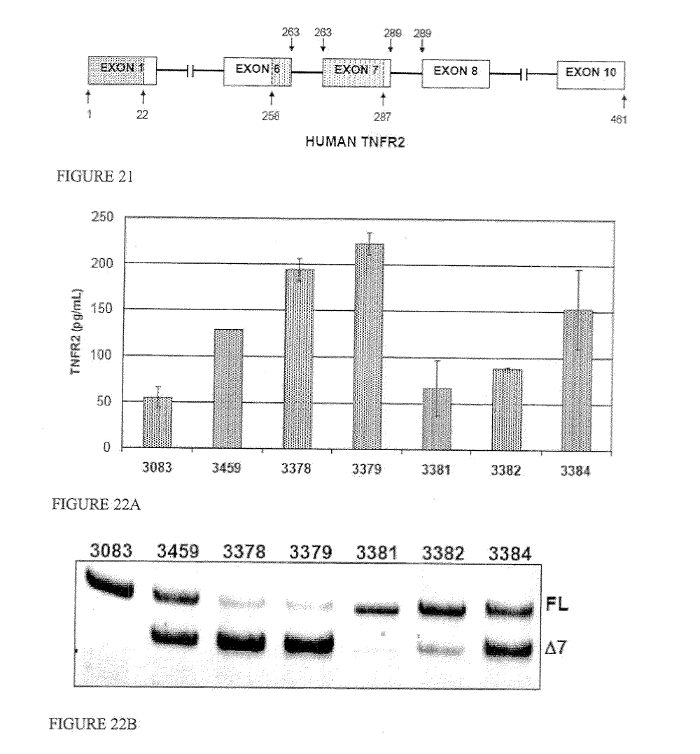

To assess the effect of SS length on splice switching activity in human TNFR2, cells were treated with SSOs of different lengths. Primary human hepatocytes were transfected with the indicated SSOs selected from Table 4. These SSOs were synthesized by Santaris Pharma, Denmark with alternating 2′deoxy- and 2′O-4′-(methylene)-bicyclic-ribonucleoside phosphorothioates. For each SSO, the 5′-terminal nucleoside was a 2′O-4′-methylene-ribonucleoside and the 3′-terminal nucleoside was a 2′deoxy-ribonucleoside. These SSOs were either 10-, 12-, 14- or 16-mers. The concentration of soluble TNFR2 Δ7 was measured by ELISA (FIG. 25, top panel). Total RNA was analyzed by RT-PCR for splice switching activity (FIG. 25, bottom panel).

### Example 14

### Analysis of the Splice Junction of SSO-induced TNFR2 Splice Variants

To confirm that the SSO splice switching, both in mice and in human cells, leads to the expected TNFR2 Δ7 mRNA, SSO-induced TNFR2 Δ7 mRNA was analyzed by RT-PCR and was sequenced.

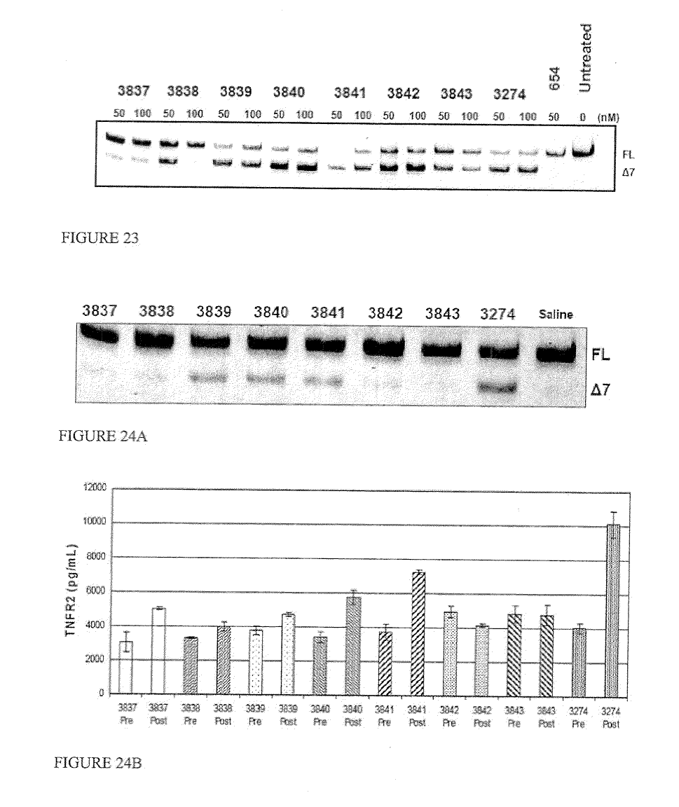

Mice. Mice were injected with SSO 3274 intraperitoneal (i.p.) at 25 mg/kg/day for 10 days. The mice were then sacrificed and total RNA from the liver was analyzed by RT-PCR using the forward primer TR045 (SEQ ID No: 228) and the reverse primer TR046 (SEQ ID No: 229). The products were analyzed on a 1.5% agarose gel (FIG. 26A) and the product for the TNFR2 Δ7 was isolated using standard molecular biology techniques. The isolated TNFR2 Δ7 product was amplified by PCR using the same primers and then sequenced (FIG. 26B). The sequence data contained the sequence CTCTCTTCCAATTGAGAAGCCCTCCTGC (nucleotides 777-804 of SEQ ID No: 127), which confirms that the SSO-induced TNFR2 Δ7 mRNA lacks exon 7 and that exon 6 is joined directly to exon 8.

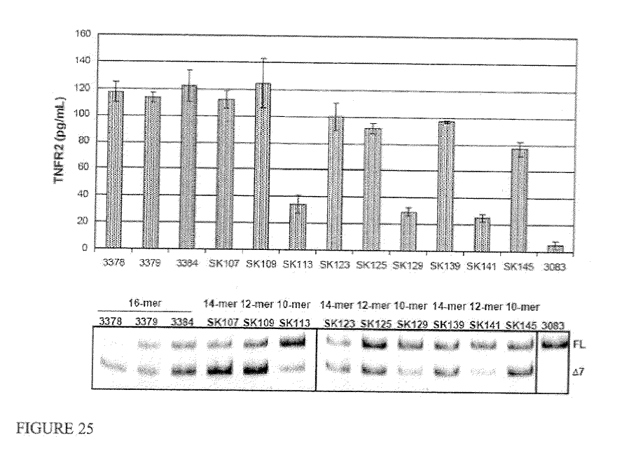

Human hepatocytes. Primary human hepatocytes were transfected with SSO 3379 as described in Example 12. Total RNA was isolated 48 hrs after transfection. The RNA was converted to cDNA with the Superscript™ II Reverse Transcriptase (Invitrogen) using random hexamer primers according to the manufacturer's directions. PCR was performed on the cDNA using the forward primer TR049 (SEQ ID No: 202) and the reverse primer TR050 (SEQ ID No: 203). The products were analyzed on a 1.5% agarose gel (FIG. 27A). The band corresponding to TNFR2 Δ7 was isolated using standard molecular biology techniques and then sequenced (FIG. 27B). The sequence data contained the sequence CGCTCTTCCAGTTGAGAAGCCCTTGTGC (nucleotides 774-801 of SEQ ID No: 125), which confirms that the SSO-induced TNFR2 Δ7 mRNA lacks exon 7 and that exon 6 is joined directly to exon 8.

### Example 15

### SSO Dose-Dependent Production of TNFR2 Δ7 Protein in Primary Human Hepatocytes

The dose response of splice-switching activity of SSOs in primary human hepatocytes was tested. Human hepatocytes were obtained in suspension from ADMET technologies. Cells were washed three times and suspended in seeding media (RPMI 1640 supplemented with L-Glut, with 10% FBS, penicillin, streptomycin, and 12 nM DexamethASONe). Hepatocytes were evaluated for viability and plated in 24-well, collagen-coated plates at 1.0×10⁵ cells per well. Typically, cell viability was 85-93%. After approximately 24 hrs, the media was replaced with maintenance media (seeding media without FBS).

For delivery of each of the SSOs to the hepatocytes, SSOs were diluted into 50 mL of OPTI-MEM™, and then 50 mL Lipofectamine™ 2000 mix (1 part Lipofectamine™ 2000 to 25 parts OPTI-MEM™) was added and incubated for 20 minutes. The SSOs were then applied to the cells in the 24-well plates. The final SSO concentration ranged from 1 to 150 nM. After 48 hrs, cells were harvested in 800 mL TRI-Reagent™.

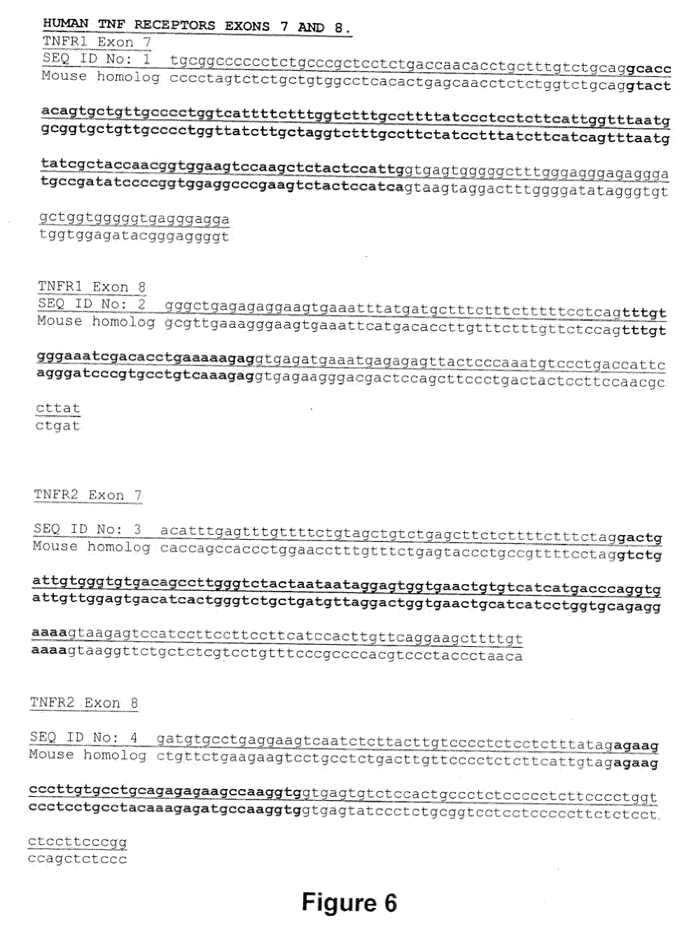

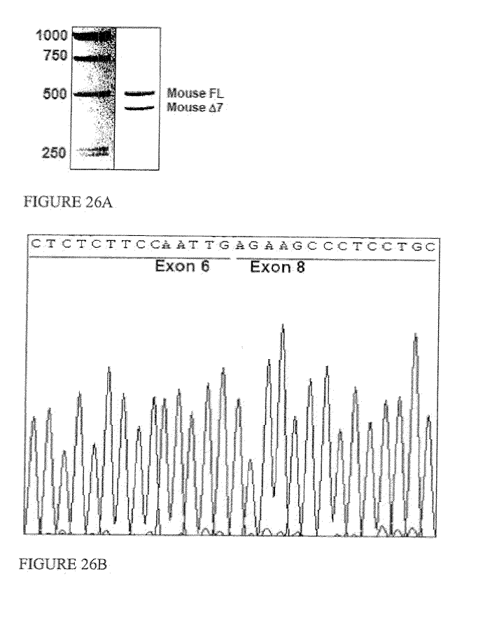

Total RNA from the cells was analyzed by RT-PCR using the forward primer TR047 (SEQ ID No: 200) and the reverse primer TR048 (SEQ ID No: 201) (FIG. 28A). The concentration of soluble TNFR2 Δ7 in the serum was measured by ELISA (FIG. 28B). Both huTNFR2 Δ7 mRNA (FIG. 8A) and secreted huTNFR2 Δ7 protein (FIG. 28B) displayed dose dependent increases.

### Example 16

### Secretion of TNFR2 Splice Variants from Murine Cells

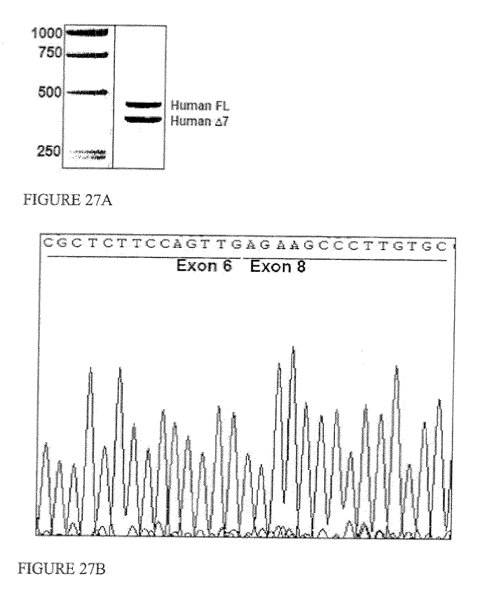

The ability of SSOs to induce soluble TNFR2 protein production and secretion into the extracellular media was tested. L929 cells were treated with SSOs as described in Example 12, and extracellular media samples were collected ˜48 hrs after transfection. The concentration of soluble TNFR2 in the samples was measured by ELISA (FIG. 29). SSOs that best induced shifts in RNA splicing, also secreted the most protein into the extracellular media. In particular, SSOs 3305, 3312, and 3274 increased soluble TNFR2 at least 3.5-fold over background. Consequently, induction of the splice variant mRNA correlated with production and secretion of the soluble TNFR2.

### Example 17

### In Vivo Injection of SSOs Generated muTNFR2 Δ7 mRNA in Mice

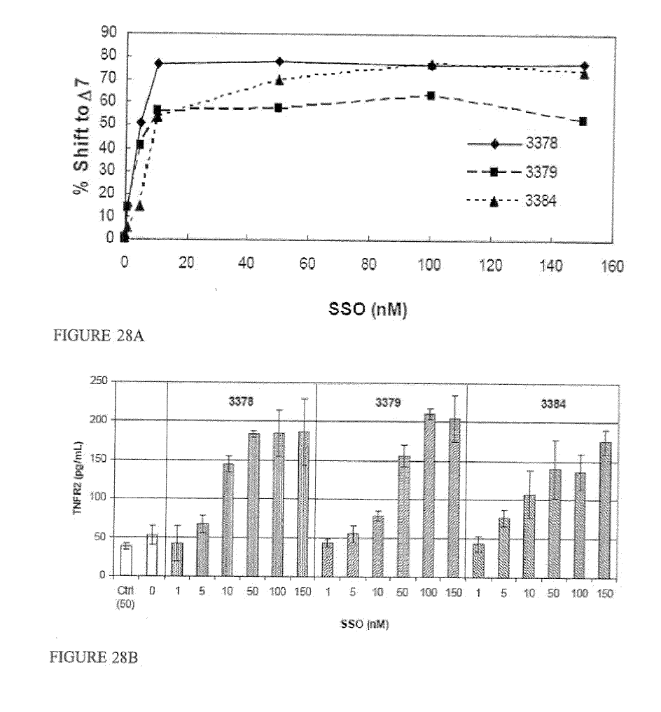

SSO 3305 in saline was injected intraperitoneal (i.p.) daily for 4 days into mice at doses from 3 mg/kg to 25 mg/kg. The mice were sacrificed on day 5 and total RNA from the liver was analyzed by RT-PCR. The data show splice switching efficacy similar to that found in cell culture. At the maximum dose of 25 mg/kg, SSO 3305 treatment induced almost full conversion to Δ7 mRNA (FIG. 30, bottom panel).

A similar experiment with SSO 3274 induced about 20% conversion to Δ7 mRNA. To optimize SSO 3274 induction of Δ7 mRNA, both the dose regimen and the time from the last injection to the sacrifice of the animal were varied. SSO 3274 was injected (i.p.) into mice daily for 4 days. SSO treatment induced about 30% conversion to Δ7 mRNA in mice analyzed on day 15, whereas a 20% shift was observed in mice analyzed on day five (FIG. 30, top panel). Furthermore, mice given Δ7 injections for 10 days, and sacrificed on day 11 showed a 50% induction of mRNA (FIG. 30, top). These in vivo data suggest that TNFR2 SSOs can produce muTNFR2 Δ7 mRNA for at least 10 days after administration.

### Example 18

### Circulatory TNFR2 Δ7

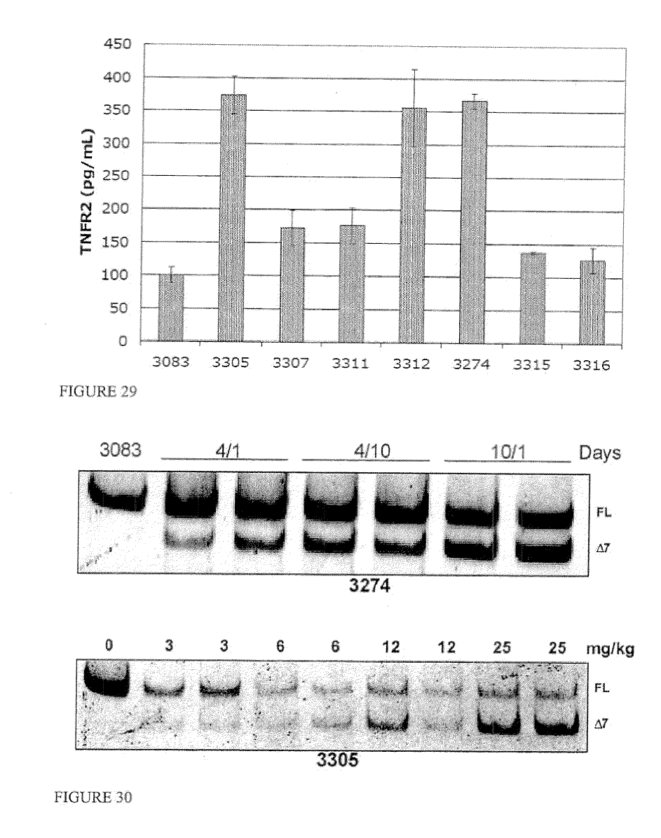

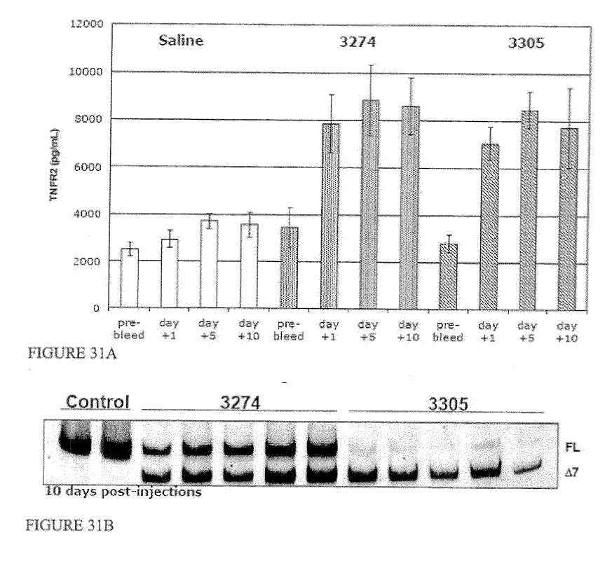

Mice were injected with SSO 3274, 3305, or the control 3083 intraperitoneal (i.p.) at 25 mg/kg/day for 10 days. Mice were bled before injection and again 1, 5 and 10 days after the last injection. The concentration of soluble TNFR2 Δ7 in the serum was measured. SSO treatment induced soluble TNFR2 Δ7 protein levels over background for at least 10 days (FIG. 31). To test the effects at longer time points, the experiment was repeated, except that serum samples were collected until day 27 after the last injection. The results show only a slight decrease in soluble TNFR2 Δ7 levels 27 days after the last SSO injection (FIG. 32).

### Example 19

### Anti-TNF-α Activity in Mice Serum

The anti-TNF-α activity of serum from SSO 3274 treated mice was tested in an L929 cytotoxicity assay. In this assay, serum is assessed for its ability to protect cultured L929 cells from the cytotoxic effects of a fixed concentration of TNF-α as described in Example 12. Serum from mice treated with SSO 3274 but not control SSOs (3083 or 3272) increased viability of the L929 cells exposed to 0.1 ng/ml, TNF-αα (FIG. 23). Hence, the SSO 3274 serum contained TNF-α antagonist sufficient to bind and to inactivate TNF-α and thereby protect the cells from the cytotoxic effects of TNF-α. This anti-TNF-α activity was present in the serum of animals 5 and 27 days after the last injection of SSO 3274.

### Example 20

### Comparison of SSO Generated TNFR2 Δ7 to Other Anti-TNF-α Antagonists

L929 cells were seeded as described above. Samples were prepared containing 90 μL of serum-free MEM, 0.1 ng/ml TNF-α and 1 μg/ml of actinomycin D, with either (i) recombinant soluble protein (0.01-3 mg/mL)) from Sigma® having the 236 amino acid residue extracellular domain of mouse TNFR2, (ii) serum from SSO 3274 or SSO 3305 treated mice (1.25-10%, diluted in serum from untreated mice; the concentration of TNFR2 Δ7 was determined by ELISA) or (iii) Enbrel® (0.45-150 pg/ml) to a final volume of 100 μl with a final mouse serum concentration of 10%. The samples were incubated at room temperature for 30 minutes. Subsequently, the samples were applied to the plated cells and incubated for ˜24 hrs at 37° C. in a 5% CO₂ humidified atmosphere. Cell viability was measured by adding 20 μL CellTiter 96® AQᵤₑₒᵤₛ One Solution Reagent (Promega) and measuring absorbance at 490 nm with a microplate reader. Cell viability was normalized to untreated cells and plotted as a function of TNF antagonist concentration (FIG. 24).

### Example 21

### Stability of TNFR2 Δ7 mRNA and Protein

Mice were treated with either SSO 3274 or 3272 (control) (n=5) by i.p. injection at a dose of 25 mg/kg/day daily for five days. Mice were bled before injection and again 5, 15, 22, 27, and 35 days after the last injection. The concentration of soluble TNFR2 Δ7 in the serum was measured (FIG. 25A). Splice shifting of TNFR2 in the liver was also determined at the time of sacrifice by RT-PCR of total RNA from the liver (FIG. 25B). Combined with data from Example 18, a time course of TNFR2 mRNA levels after SSO treatment was constructed, and compared with the time course of TNFR2 Δ7 protein in serum (FIG. 26). The data show that TNFR2 Δ7 mRNA in vivo decays at a rate approximately 4 times faster than that of TNFR2 Δ7 protein in serum. On day 35, TNFR2 Δ7 mRNA was only detectable in trace amounts, whereas TNFR2 Δ7 protein had only decreased by 20% from its peak concentration.

### Example 22

### Generation of Human TNFR2 Δ7 cDNA

A plasmid containing the full length human TNFR2 cDNA was obtained commercially from OriGene (Cat. No: TC119459, NM_{—}001066.2). The cDNA was obtained by performing PCR on the plasmid using reverse primer TR001 (SEQ ID No: 116) and forward primer TR002 (SEQ ID No: 1117). The PCR product was isolated and was purified using standard molecular biology techniques, and contains the 1383 bp TNFR2 open reading frame without a stop codon.

Alternatively, full length human TNFR2 cDNA is obtained by performing RT-PCR on total RNA from human mononuclear cells using the TR001 reverse primer and the TR002 forward primer. The PCR product is isolated and is purified using standard molecular biology techniques.

To generate human TNFR2 Δ7 cDNA, two separate PCR reactions were performed on the full length human TNFR2 cDNA, thereby creating overlapping segments of the TNFR2 Δ7 cDNA. In one reaction, PCR was performed on full length TNFR2 cDNA using the forward primer TR003 (SEQ ID No: 190) and the reverse primer TR004 (SEQ ID No: 191). In the other reaction, PCR was performed on full length TNFR2 cDNA using the reverse primer TR005 (SEQ ID No: 192) and the TR002 forward primer. Finally, the 2 overlapping segments were combined, and PCR was performed using the TR002 forward primer and the TR004 reverse primer. The PCR product was isolated and was purified using standard molecular biology techniques, and was expected to contain the 1308 bp TNFR2 Δ7 open reading frame with a stop codon (SEQ ID No: 125).

Similarly, by using the TR001 reverse primer instead of the TR004 reverse primer in these PCR reactions the 1305 bp human TNFR2 Δ7 open reading frame without a stop codon was generated. This allows for the addition of in-frame C-terminal affinity purification tags, such as His-tag, when the final PCR product is inserted into an appropriate vector.

### Example 23

### Generation of Human TNFR1Δ7 cDNA

A plasmid containing the full length human TNFR2 cDNA is obtained commercially from OriGene (Cat. No: TC127913, NM_{—}001065.2). The cDNA is obtained by performing PCR on the plasmid using the TR006 reverse primer (SEQ ID No: 204) and the TR007 forward primer (SEQ ID No: 205). The full length human TNFR1 cDNA PCR product is isolated and is purified using standard molecular biology techniques.

Alternatively, full length human TNFR1 cDNA is obtained by performing RT-PCR on total RNA from human mononuclear cells using the TR006 reverse primer and the TR007 forward primer. The full length human TNFR1 cDNA PCR product is isolated and is purified using standard molecular biology techniques.

To generate human TNFR1 Δ7 cDNA, two separate PCR reactions are performed on the full length human TNFR1 cDNA, thereby creating overlapping segments of the TNFR1 Δ7 cDNA. In one reaction, PCR is performed on full length TNFR1 cDNA using the TR008 forward primer (SEQ ID No: 206) and the TR006 reverse primer. In the other reaction, PCR is performed on full length TNFR1 cDNA using the TR009 reverse primer (SEQ ID No: 207) and the TR010 forward primer (SEQ ID No: 208). Finally, the 2 overlapping segments are combined, and PCR is performed using the TR010 forward primer and the TR006 reverse primer. The PCR product is isolated and is purified using standard molecular biology techniques, and contains the 1254 bp human TNFR1 Δ7 open reading frame with a stop codon (SEQ ID No: 121).

Alternatively, by using the TR011 reverse primer (SEQ ID No: 209) instead of the TR006 reverse primer in these PCR reactions the 1251 bp human TNFR1 Δ7 open reading frame without a stop codon is generated. This allows for the addition of in-frame C-terminal affinity purification tags, such as His-tag, when the final PCR product is inserted into an appropriate vector.

### Example 24

### Generation of Murine TNFR2 Δ7 cDNA

To generate full length murine TNFR2 cDNA, PCR was performed on the commercially available FirstChoice™ PCR-Ready Mouse Liver cDNA (Ambion, Cat. No: AM3300) using the TR012 reverse primer (SEQ ID No: 214) and the TR013 forward primer (SEQ ID No: 215). The full length murine TNFR2 cDNA PCR product is isolated and is purified using standard molecular biology techniques. Then by performing PCR on the resulting product using the TR014 forward primer (SEQ ID No: 216) and the TR012 reverse primer the proper Kozak sequence was introduced.

Alternatively, full length murine TNFR2 cDNA is obtained by performing RT-PCR on total RNA from mouse mononuclear cells or mouse hepatocytes using the TR015 reverse primer (SEQ ID No: 217) and the TR016 forward primer (SEQ ID No: 218). The full length murine TNFR2 cDNA PCR product is isolated and is purified using standard molecular biology techniques.

To generate murine TNFR2 Δ7 cDNA, two separate PCR reactions were performed on the full length murine TNFR2 cDNA, thereby creating overlapping segments of the TNFR2 Δ7 cDNA. In one reaction, PCR was performed on full length TNFR2 cDNA using the TR017 forward primer (SEQ ID No: 219) and the TR015 reverse primer. In the other reaction, PCR was performed on full length TNFR2 cDNA using the TR018 reverse primer (SEQ ID No: 220) and the TR016 forward primer. Finally, the 2 overlapping segments were combined, and PCR was performed using the TR016 forward primer and the TR015 reverse primer. The PCR product was isolated and was purified using standard molecular biology techniques, and was expected to contain the 1348 bp murine TNFR2 Δ7 open reading frame with a stop codon (SEQ ID No: 127).

Alternatively, by using the TR019 reverse primer (SEQ ID No: 221) instead of the TR015 reverse primer in these PCR reactions the 1345 bp murine TNFR2 Δ7 open reading frame without a stop codon was generated. This allows for the addition of in-frame C-terminal affinity purification tags, such as His-tag, when the final PCR product is inserted into an appropriate vector.

### Example 25

### Generation of Murine TNFR1 Δ7 cDNA

To generate full length murine TNFR1 cDNA, PCR is performed on the commercially available FirstChoice™ PCR-Ready Mouse Liver cDNA (Ambion, Cat. No: AM3300) using the TR020 reverse primer (SEQ ID No: 230) and the TR021 forward primer (SEQ ID No: 231). The full length murine TNFR1 cDNA PCR product is isolated and is purified using standard molecular biology techniques.

Alternatively, full length murine TNFR1 cDNA is obtained by performing RT-PCR on total RNA from mouse mononuclear cells using the TR020 reverse primer and the TR021 forward primer. The full length murine TNFR1 cDNA PCR product is isolated and is purified using standard molecular biology techniques.

To generate murine TNFR1 Δ7 cDNA, two separate PCR reactions are performed on the full length human TNFR1 cDNA, thereby creating overlapping segments of the TNFR1 Δ7 cDNA. In one reaction, PCR is performed on full length TNFR1 cDNA using the TR022 forward primer (SEQ ID No: 232) and the TR020 reverse primer. In the other reaction, PCR is performed on full length TNFR1 cDNA using the TR023 reverse primer (SEQ ID No: 233) and the TR024 forward primer (SEQ ID No: 234). Finally, the 2 overlapping segments are combined, and PCR is performed using TR024 forward primer and the TR020 reverse primer. The 1259 bp PCR product is isolated and is purified using standard molecular biology techniques, and contains the 1251 bp murine TNFR1 Δ7 open reading frame with a stop codon (SEQ ID No: 123).

Alternatively, by using the TR025 reverse primer (SEQ ID No: 235) instead of the TR020 reverse primer in these PCR reactions the 1248 bp murine TNFR1 Δ7 open reading frame without a stop codon is generated. This allows for the addition of in-frame C-terminal affinity purification tags, such as His-tag, when the final PCR product is inserted into an appropriate vector.

### Example 26

### Construction of Vectors for the Expression of Human TNFR2 Δ7 in Mammalian Cells

For expression of the human TNFR2 Δ7 protein in mammalian cells, a human TNFR2 Δ7 cDNA PCR product from Example 23 was incorporated into an appropriate mammalian expression vector. The TNFR2 Δ7 cDNA PCR product from Example 23, both with and without a stop codon, and the pcDNA™3.1 D/V5-His TOPO® expression vector (Invitrogen) were blunt-end ligated and isolated according to the manufacturer's directions. Plasmids containing inserts encoding human TNFR2 Δ7 were transformed into OneShot® Top10 competent cells (Invitrogen), according to the supplier's directions. Fifty mL of the transformation mix were plated on LB media with 100 mg/mL of ampicillin and incubated overnight at 37° C. Single colonies were used to inoculate 5 mL cultures of LB media with 100 mg/mL ampicillin and incubated overnight at 37° C. The cultures were then used to inoculate 200 mL of LB media with 100 mg/mL of ampicillin and grown overnight at 37° C. The plasmids were isolated using GenElute™ Plasmid Maxiprep kit (Sigma) according to manufacturer's directions. Purification efficiency ranged from 0.5 to 1.5 mg of plasmid per preparation.

Three human TNFR2 Δ7 clones (1319-1, 1138-5 and 1230-1) were generated and sequenced. Clone 1319-1 contains the human TNFR2 Δ7 open reading frame without a stop codon followed directly by an in-frame His-tag from the plasmid; while clones 1138-5 and 1230-1 contain the TNFR2 Δ7 open reading frame followed immediately by a stop codon. The sequence of the His-tag from the plasmid is given in SEQ ID No: 242. The sequences of the TNFR2 Δ7 open reading frames of clones 1230-1 and 1319-1 were identical to SEQ ID No: 125 with and without the stop codon, respectively. However relative to SEQ ID No: 125, the sequence (SEQ ID No: 231) of the TNFR2 Δ7 open reading frames of clone 1138-5 differed by a single nucleotide at position 1055 in exon 10, with an A in the former and a G in the later. This single nucleotide change causes the amino acid 352 to change from a glutamine to an arginine.

### Example 27

### Expression of Human TNFR2 Δ7 in E. coli

For expression of the human TNFR2 Δ7 protein in bacteria, a human TNFR2 Δ7 cDNA from Example 23 is incorporated into an appropriate expression vector, such as a pET Directional TOPO® expression vector (Invitrogen). PCR is performed on the PCR fragment from Example 23 using forward (TR002) (SEQ ID No: 191) and reverse (TR026) (SEQ ID No: 195) primers to incorporate a homologous recombination site for the vector. The resulting PCR fragment is incubated with the pET101/D-TOPO® vector (Invitrogen) according to the manufacturer's directions, to create the human TNFR2 Δ7 bacterial expression vector. The resulting vector is transformed into the E. coli strain BL21 (DE3). The human TNFR2 Δ7 is then expressed from the bacterial cells according to the manufacturer's instructions.

### Example 28

### Expression of Human TNFR2 Δ7 in Insect Cells

For expression of the human TNFR2 Δ7 protein in insect cells, a human TNFR2 Δ7 cDNA from Example 23 is incorporated into a baculoviral vector. PCR is performed on a human TNFR2 Δ7 cDNA from Example 23 using forward (TR027) (SEQ ID No: 196) and reverse (TR028) (SEQ ID No: 197) primers. The resulting PCR product is digested with the restriction enzymes EcoRI and XhoI. The digested PCR product is ligated with a EcoRI and XhoI digested pENTR™ Vector (Invitrogen), such as any one of the pENTR™1A, pENTR™2B, pENTR™3C, pENTR™4, or pENTR™11 Vectors, to yield an entry vector. The product is then isolated, amplified, and purified using standard molecular biology techniques.

A baculoviral vector containing the human TNFR2 Δ7 cDNA is generated by homologous recombination of the entry vector with BaculoDirect™ Linear DNA (Invitrogen) using LR Clonase™ (Invitrogen) according to the manufacturer's directions. The reaction mixture is then used to infect Sf9 cells to generate recombinant baculovirus. After harvesting the recombinant baculovirus, expression of human TNFR2 Δ7 is confirmed. Amplification of the recombinant baculovirus yields a high-titer viral stock. The high-titer viral stock is used to infect Sf9 cells, thereby expressing human TNFR2 Δ7 protein.

### Example 29

### Generation of Adeno-Associated Viral Vectors for the Expression of Human TNFR2 Δ7

For in vitro or in vivo delivery to mammalian cells of the human TNFR2 Δ7 gene for expression in those mammalian cells, a recombinant adeno-associated virus (rAAV) vector is generated using a three plasmid transfection system as described in Grieger, J., et al., 2006, Nature Protocols 1:1412. PCR is performed on a purified human TNFR2 D7 PCR product of Example 23 using forward (TR029) (SEQ ID No: 198) and reverse (TR030) (SEQ ID No: 199) primers to introduce unique flanking NotI restriction sites. The resulting PCR product is digested with the NotI restriction enzyme, and isolated by standard molecular biology techniques. The NotI-digested fragment is then ligated to NotI-digested pTR-UF2 (University of North Carolina (UNC) Vector Core Facility), to create a plasmid that contains the human TNFR2 D7 open reading frame, operably linked to the CMVie promoter, flanked by inverted terminal repeats. The resulting plasmid is then transfected with the plasmids pXX680 and pHelper (UNC Vector Core Facility) into HEK-293 cells, as described in Grieger, J., et al., to produce rAAV particles containing the human TNFR2 Δ7 gene where expression is driven by the strong constitutive CMVie promoter. The virus particles are harvested and purified, as described in Grieger, J., et al., to provide an rAAV stock suitable for transducing mammalian cells.

### Example 30

### Expression of Human TNFR1 Δ7 in E. coli

For expression of the human TNFR1 Δ7 protein in bacteria, the cDNA is incorporated into an appropriate expression vector, such as a pET Directional TOPO® expression vector (Invitrogen). PCR is performed on the cDNA using forward (TR010) (SEQ ID No: 208) and reverse (TR006) (SEQ ID No: 204) primers to incorporate a homologous recombination site for the vector. The resulting PCR fragment is incubated with the pET101/D-TOPO® vector (Invitrogen) according to the manufacturer's directions, to create the human TNFR1 Δ7 bacterial expression vector. The resulting vector is transformed into the E. coli strain BL21 (DE3). The human TNFR1 Δ7 is then expressed from the bacterial cells according to the manufacturer's instructions.

### Example 31

### Expression of Human TNFR1 Δ7 in Mammalian Cells

For expression of the human TNFR1 Δ7 protein in mammalian cells, a human TNFR1 Δ7 cDNA PCR product is incorporated into an appropriate mammalian expression vector. human TNFR1 Δ7 cDNA PCR product and the pcDNA™3.1 D/V5-His TOPO® expression vector (Invitrogen) are blunt-end ligated according to the manufacturer's directions. The product is then isolated, amplified, and purified using standard molecular biology techniques to yield the mammalian expression vector. The vector is then transfected into a mammalian cell, where expression of the human TNFR1 Δ7 protein is driven by the strong constitutive CMVie promoter.

### Example 32

### Expression of Human TNFR1 Δ7 in Insect Cells

For expression of the human TNFR1 Δ7 protein in insect cells, the cDNA from Example 33 is incorporated into a baculoviral vector. PCR is performed on the cDNA from Example 33 using forward (TR031) (SEQ ID No: 210) and reverse (TR032) (SEQ ID No: 211) primers. The resulting PCR product is digested with the restriction enzymes EcoRI and XhoI. The digested PCR product is ligated with a EcoRI and XhoI digested pENTR™ Vector (Invitrogen), such as any one of the pENTR™1A, pENTR™2B, pENTR™3C, pENTR™4, or pENTR™11 Vectors, to yield an entry vector. The product is then isolated, amplified, and purified using standard molecular biology techniques.

A baculoviral vector containing the human TNFR1 Δ7 cDNA is generated by homologous recombination of the entry vector with BaculoDirect™ Linear DNA (Invitrogen) using LR Clonase™ (Invitrogen) according to the manufacturer's directions. The reaction mixture is then used to infect Sf9 cells to generate recombinant baculovirus. After harvesting the recombinant baculovirus, expression of human TNFR1 Δ7 is confirmed. Amplification of the recombinant baculovirus yields a high-titer viral stock. The high-titer viral stock is used to infect Sf9 cells, thereby expressing human TNFR1 Δ7 protein.

### Example 33

### Generation of Adeno-Associated Viral Vectors for the Expression of Human TNFR1 Δ7

For in vitro or in vivo delivery to mammalian cells of the human TNFR1 Δ7 gene for expression in those mammalian cells, a recombinant adeno-associated virus (rAAV) vector is generated using a three plasmid transfection system as described in Grieger, J., et al., 2006, Nature Protocols 1:1412. PCR is performed on the purified human TNFR1 D7 PCR product using forward (TR033) (SEQ ID No: 212) and reverse (TR034) (SEQ ID No: 213) primers to introduce unique flanking NotI restriction sites. The resulting PCR product is digested with the NotI restriction enzyme, and isolated by standard molecular biology techniques. The NotI-digested fragment is then ligated to NotI-digested pTR-UF2 (University of North Carolina (UNC) Vector Core Facility), to create a plasmid that contains the human TNFR1 D7 open reading frame, operably linked to the CMVie promoter, flanked by inverted terminal repeats. The resulting plasmid is then transfected with the plasmids pXX680 and pHelper (UNC Vector Core Facility) into HEK-293 cells, as described in Grieger, J., et al., to produce rAAV particles containing the human TNFR1 Δ7 gene where expression is driven by the strong constitutive CMVie promoter. The virus particles are harvested and purified, as described in Grieger, J., et al., to provide an rAAV stock suitable for transducing mammalian cells.

### Example 34

### Construction of Vectors for the Expression of Mouse TNFR2 Δ7 in Mammalian Cells

For expression of the murine TNFR2 Δ7 protein in mammalian cells, a murine TNFR2 Δ7 cDNA PCR product from Example 25 was incorporated into an appropriate mammalian expression vector. The TNFR2 Δ7 cDNA PCR product from Example 25 both with and without a stop codon, and the pcDNA™3.1D/V5-His TOPO® expression vector (Invitrogen) was blunt-end ligated and isolated according to the manufacturer's directions. Plasmids containing inserts encoding murine Δ7 TNFR2 were transformed into OneShot® Top10 competent cells (Invitrogen), according to the supplier's directions. Fitly mL of the transformation mix were plated on LB media with 100 mg/mL of ampicillin and incubated overnight at 37° C. Single colonies were used to inoculate 5 mL cultures of LB media with 100 mg/mL ampicillin and incubated overnight at 37° C. The cultures were then used to inoculate 200 mL of LB media with 100 mg/mL of ampicillin and grown overnight at 37° C. The plasmids were isolated using GenElute™ Plasmid Maxiprep kit (Sigma) according to manufacturer's directions. Purification efficiency ranged from 0.5 to 1.5 mg of plasmid per preparation.

Two murine TNFR2 Δ7 clones (1144-4 and 1145-3) were generated and sequenced. Clone 1144-4 contains the murine TNFR2 Δ7 open reading frame without a stop codon followed directly by an in-frame His-tag from the plasmid; while clone 1145-3 contains the TNFR2 Δ7 open reading frame followed immediately by a stop codon. The sequence of the His-tag from the plasmid is given in SEQ ID No: 242. Relative to SEQ ID No: 127, the sequence (SEQ ID No: 240) of the TNFR2 Δ7 open reading frames of the two clones, 1144-4 and 1145-3, differed by a single nucleotide at eleven positions. As a result of these single nucleotide changes there are four amino acid differences relative to SEQ ID No: 128.

### Example 35

### Expression of Murine TNFR2 Δ7 in E. coli

For expression of the mouse TNFR2 Δ7 protein in bacteria, a murine TNFR2 Δ7 cDNA from Example 25 is incorporated into an appropriate expression vector, such as a pET Directional TOPO® expression vector (Invitrogen). PCR is performed on the PCR fragment from Example 25 using forward (TR035) (SEQ ID No: 222) and reverse (TR036) (SEQ ID No: 223) primers to incorporate a homologous recombination site for the vector. The resulting PCR fragment is incubated with the pET101/D-TOPO® vector (Invitrogen) according to the manufacturer's directions, to create the murine TNFR2 Δ7 bacterial expression vector. The resulting vector is transformed into the E. coli strain BL21 (DE3). The murine TNFR2 Δ7 is then expressed from the bacterial cells according to the manufacturer's instructions.

### Example 36

### Expression of Mouse TNFR2 Δ7 in Insect Cells

For expression of the murine TNFR2 Δ7 protein in insect cells, the cDNA from Example 25 is incorporated into a baculoviral vector. PCR is performed on the cDNA from Example 25 using forward (TR037) (SEQ ID No: 224) and reverse (TR038) (SEQ ID No: 225) primers. The resulting PCR product is digested with the restriction enzymes EcoRI and XhoI. The digested PCR product is ligated with a EcoRI and XhoI digested pENTR™ Vector (Invitrogen), such as any one of the pENTR™1A, pENTR™2B, pENTR™3C, pENTR™4, or pENTR™11 Vectors, to yield an entry vector. The product is then isolated, amplified, and purified using standard molecular biology techniques.

A baculoviral vector containing the murine TNFR2 Δ7 cDNA is generated by homologous recombination of the entry vector with BaculoDirect™ Linear DNA (Invitrogen) using LR Clonase™ (Invitrogen) according to the manufacturer's directions. The reaction mixture is then used to infect Sf9 cells to generate recombinant baculovirus. After harvesting the recombinant baculovirus, expression of murine TNFR2 Δ7 is confirmed. Amplification of the recombinant baculovirus yields a high-titer viral stock. The high-titer viral stock is used to infect Sf9 cells, thereby expressing murine TNFR2 Δ7 protein.

### Example 37

### Generation of Adeno-Associated Viral Vectors for the Expression of Murine TNFR2 Δ7

For in vitro or in vivo delivery to mammalian cells of the murine TNFR2 Δ7 gene for expression in those mammalian cells, a recombinant adeno-associated virus (rAAV) vector is generated using a three plasmid transfection system as described in Grieger, J., et al., 2006, Nature Protocols 1-1412. PCR is performed on the purified murine TNFR2 D7 PCR product of Example 25 using forward (TR039) (SEQ ID No: 226) and reverse (TR040) (SEQ ID No: 227) primers to introduce unique flanking NotI restriction sites. The resulting PCR product is digested with the NotI restriction enzyme, and isolated by standard molecular biology techniques. The NotI-digested fragment is then ligated to NotI-digested pTR-UF2 (University of North Carolina (UNC) Vector Core Facility), to create a plasmid that contains the murine TNFR2 D7 open reading frame, operably linked to the CMVie promoter, flanked by inverted terminal repeats. The resulting plasmid is then transfected with the plasmids pXX680 and pHelper (UNC Vector Core Facility) into HEK-293 cells, as described in Grieger, J., et al., to produce rAAV particles containing the murine TNFR2 Δ7 gene where expression is driven by the strong constitutive CMVie promoter. The virus particles are harvested and purified, as described in Grieger, J., et al., to provide a rAAV stock suitable for transducing mammalian cells.

### Example 38

### Expression of Murine TNFR1 Δ7 in E. coli

For expression of the mouse TNFR1 Δ7 protein in bacteria, the cDNA from Example 26 is incorporated into an appropriate expression vector, such as a pET Directional TOPO® expression vector (Invitrogen). PCR is performed on the cDNA from Example 26 using forward (TR024) (SEQ ID No: 234) and reverse (TR020) (SEQ ID No: 235) primers to incorporate a homologous recombination site for the vector. The resulting PCR fragment is incubated with the pET101/D-TOPO® vector (Invitrogen) according to the manufacturer's directions, to create the murine TNFR1 Δ7 bacterial expression vector. The resulting vector is transformed into the E. coli strain BL21 (DE3). The murine TNFR1 Δ7 is then expressed from the bacterial cells according to the manufacturer's instructions.

### Example 39

### Expression of Mouse TNFR1 Δ7 in Mammalian Cells

For expression of the murine TNFR1 Δ7 protein in mammalian cells, a murine TNFR1 Δ7 cDNA PCR product from Example 26 is incorporated into an appropriate mammalian expression vector. The murine TNFR1 Δ7 cDNA PCR product from Example 26 and the pcDNA™3.1 D/V5-His TOPO® expression vector (Invitrogen) are blunt-end ligated according to the manufacturer's directions. The product is then isolated, amplified, and purified using standard molecular biology techniques to yield the mammalian expression vector. The vector is then transfected into a mammalian cell, where expression of the murine TNFR1 Δ7 protein is driven by the strong constitutive CMVie promoter.

### Example 40

### Expression of Mouse TNFR1 Δ7 in Insect Cells

For expression of the murine TNFR1 Δ7 protein in insect cells, the cDNA from Example 26 is incorporated into a baculoviral vector. PCR is performed on the cDNA from Example 26 using forward (TR041) (SEQ ID No: 236) and reverse (TR042) (SEQ ID No: 237) primers. The resulting PCR product is digested with the restriction enzymes EcoRI and XhoI. The digested PCR product is ligated with a EcoRI and XhoI digested pENTR™ Vector (Invitrogen), such as any one of the pENTR™1A, pENTR™2B, pENTR™3C, pENTR™4, or pENTR™11 Vectors, to yield an entry vector. The product is then isolated, amplified, and purified using standard molecular biology techniques.

A baculoviral vector containing the murine TNFR1 Δ7 cDNA is generated by homologous recombination of the entry vector with BaculoDirect™ Linear DNA (Invitrogen) using LR Clonase™ (Invitrogen) according to the manufacturer's directions. The reaction mixture is then used to infect Sf9 cells to generate recombinant baculovirus. After harvesting the recombinant baculovirus, expression of murine TNFR1 Δ7 is confirmed. Amplification of the recombinant baculovirus yields a high-titer viral stock. The high-titer viral stock is used to infect Sf9 cells, thereby expressing murine TNFR1 Δ7 protein.

### Example 41

### Generation of Adeno-Associated Viral Vectors for the Expression of Murine TNFR1 Δ7

For in vitro or in vivo delivery to mammalian cells of the murine TNFR1 Δ7 gene for expression in those mammalian cells, a recombinant adeno-associated virus (rAAV) vector is generated using a three plasmid transfection system as described in Grieger, J., et al., 2006, Nature Protocols 1:1412. PCR is performed on the purified murine TNFR1 D7 PCR product of Example 25, using forward (TR043) (SEQ ID No: 238) and reverse (TR044) (SEQ ID No: 239) primers to introduce unique flanking NotI restriction sites. The resulting PCR product is digested with the NotI restriction enzyme, and isolated by standard molecular biology techniques. The NotI-digested fragment is then ligated to NotI-digested pTR-UF2 (University of North Carolina (UNC) Vector Core Facility), to create a plasmid that contains the murine TNFR1 D7 open reading frame, operably linked to the CMVie promoter, flanked by inverted terminal repeats. The resulting plasmid is then transfected with the plasmids pXX680 and pHelper (UNC Vector Core Facility) into HEK-293 cells, as described in Grieger, J., et al., to produce rAAV particles containing the murine TNFR1 Δ7 gene where expression is driven by the strong constitutive CMVie promoter. The virus particles are harvested and purified, as described in Grieger, J., et al., to provide an rAAV stock suitable for transducing mammalian cells.

### Example 42

### Generation of Lentiviral Vectors for the Expression of TNFR Δ7

For in vitro or in vivo delivery to mammalian cells of a TNFR Δ7 gene for expression in those mammalian cells, a replication-incompetent lentivirus vector is generated. A PCR product from Examples 27, 30, 35 and 38 and the pLenti6/V5-D-TOPO® vector (Invitrogen) are blunt-end ligated according to the manufacturer's directions. The resulting plasmid is transformed into E. coli, amplified, and purified using standard molecular biology techniques. This plasmid is transfected into 293 FT cells (Invitrogen) according to the manufacturer's directions to produce lentivirus particles containing the TNFR Δ7 gene where expression is driven by the strong constitutive CMVie promoter. The virus particles are harvested and purified, as described in Tiscornia, G., et al., 2006, Nature Protocols 1:241, to provide a lentiviral stock suitable for transducing mammalian cells.

### Example 43

### Expression of TNFR2 Δ7 in Mammalian Cells

The plasmids generated in Examples 26 and 34 were used to express active protein in mammalian HeLa cells, and the resulting proteins were tested for anti-TNF-α activity. HeLa cells were seeded in at 1.0×10⁵ cells per well in 24-well plates in SMEM media containing L-glutamine, gentamicin, kanamycin, 5% FBS and 5% HS. Cells were grown overnight at 37° C. in a 5% CO₂ humidified atmosphere. Approximately 250 ng of plasmid DNA was added to 50 mL of OPTI-MEM™, and then 50 ml Lipofectamine™ 2000 mix (1 part Lipofectamine™ 2000 to 25 parts OPTI-MEM™) was added and incubated for 20 minutes. Then 400 mL of serum free media was added and then applied to the cells in the 24-well plates. After incubation for ˜48 hrs at 37° C. in a 5% CO₂ humidified atmosphere, the media was collected and the cells were harvested in 800 mL TRI-Reagent™. Total RNA was isolated from the cells per the manufacturer's directions and analyzed by RT-PCR using the forward primer TR047 (SEQ ID No: 200) and the reverse primer TR048 (SEQ ID No: 201) for human TNFR2 Δ7, or the forward primer TR045 (SEQ ID No: 228) and the reverse primer TR046 (SEQ ID No: 229) for mouse TNFR2 Δ7. The concentration of soluble TNFR2 in the media was measured by ELISA.

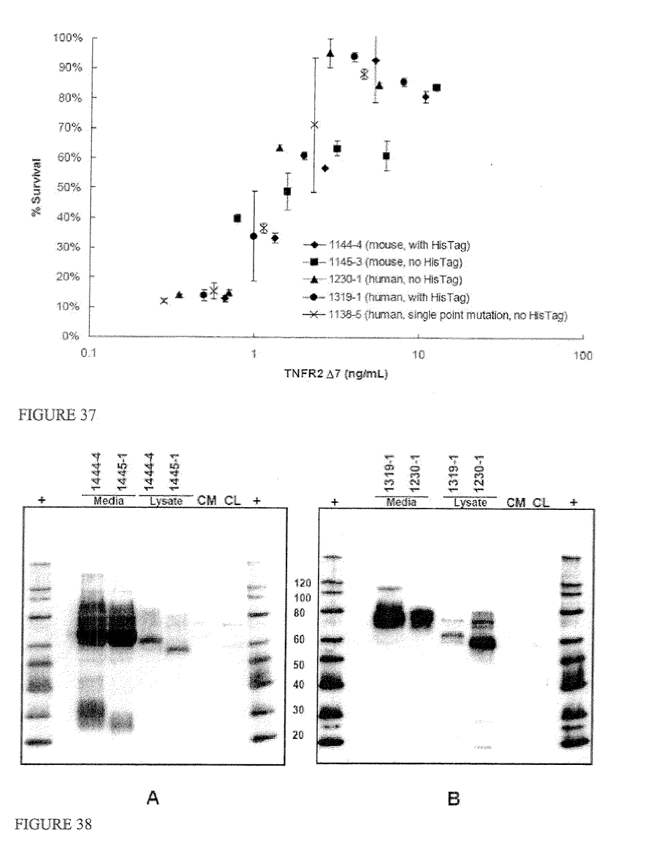

The anti-TNF-α activity of the above media was tested in an L929 cytotoxicity assay. L929 cells were plated in 96-well plates at 2×10⁴ cells per well in MEM media containing 10% regular FBS, penicillin and streptomycin and grown overnight at 37° C. in a 5% CO₂ humidified atmosphere. The media samples were diluted 1, 2, 4, 8 and 16 fold with media from non-transfected HeLa cells. Ninety μL of each of these samples was added to 10 μL of serum-free media, containing 1.0 ng/ml TNF-α and 1 μg/ml of actinomycin D. The media from the cells were removed and replaced with these 100 μL samples. The cells were then grown overnight at 37° C. in a 5% CO₂ humidified atmosphere. Twenty mL CellTiter 96® AQᵤₑₒᵤₛ One Solution Reagent (Promega) was then added to each well. Cell viability was measured 4 hrs later by measuring absorbance at 490 nm with a microplate reader. Cell viability was normalized to untreated cells and plotted as a function of TNF antagonist concentration (FIG. 37).

The data from this example and from Example 20 were analyzed using the GraphPad Prism® software to determine the EC₅₀ value for each antagonist. For each antagonist from these examples a sigmoidal dose-response curve was fit by non-linear regression with the maximum and minimum responses held fixed to 100% and 0%, respectively. The EC₅₀ values shown in Table 8 correspond to a 95% confidence level, and each curve had an r value ranging from 0.7 to 0.9.

TABLE 8
Activity of TNF-α antagonists

| | EC₅₀ |
|---|---|
| TNF-α Antagonist | (ng/mL) |
| Etanercept | 1.1 ± 0.5 |
| Recombinant soluble TNFR2 (rsTNFR2) | 698 ± 180 |
| SSO 3305 treated mice serum (mouse TNFR2 Δ7) | 0.6 ± 0.2 |
| SSO 3274 treated mice serum (mouse TNFR2 Δ7) | 0.8 ± 0.3 |
| Extracellular media from 1144-4 transfected HeLa cells | 2.4 ± 1.4 |
| (mouse TNFR2 Δ7) | |
| Extracellular media from 1145-3 transfected HeLa cells | 2.4 ± 0.8 |
| (mouse TNFR2 Δ7) | |
| Extracellular media from 1230-1 transfected HeLa cells | 1.4 ± 1.1 |
| (human TNFR2 Δ7) | |
| Extracellular media from 1319-1 transfected HeLa cells | 1.7 ± 1.0 |
| (human TNFR2 Δ7) | |
| Extracellular media from 1138-5 transfected HeLa cells | 1.8 ± 1.1 |
| (human TNFR2 Δ7) | |

### Example 44

### Expression and Purification of TNFR2 Δ7 in Mammalian Cells

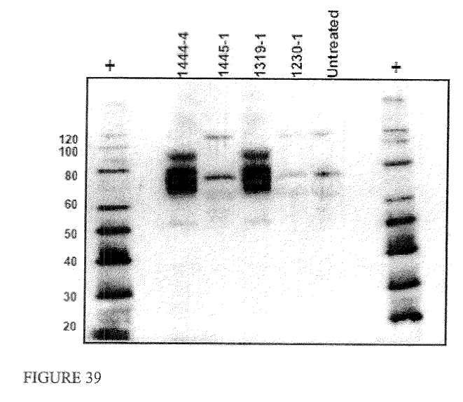

The plasmids generated in Example 26 and Example were used to express and purify TNFR2 Δ7 from mammalian HeLa cells. HeLa cells were plated in 6-well plates at 5×10⁵ cells per well, and grown overnight at 37° C., 5% CO₂, in humidified atmosphere. Each well was then transfected with 1.5 mg of plasmid DNA using either 1144-4 (mouse TNFR2 Δ7 with His-tag), 1145-1 (mouse TNFR2 Δ7, no His-tag), 1230-1 (human TNFR2 Δ7, no His-tag) or 1319-1 (human TNFR2 Δ7 with His-tag) plasmids. Media was collected ˜48 hrs after transfection and concentrated approximately 40-fold using Amicon MWCO 30,000 filters. The cells were lysed in 120 mL of RIPA lysis buffer (Invitrogen) with protease inhibitors (Sigma-aldrich) for 5 minutes on ice. Protein concentration was determined by the Bradford assay. Proteins were isolated from aliquots of the cell lysates and the extracellular media and analyzed by western blot for TNFR2 as described in Example 12 (FIG. 38).

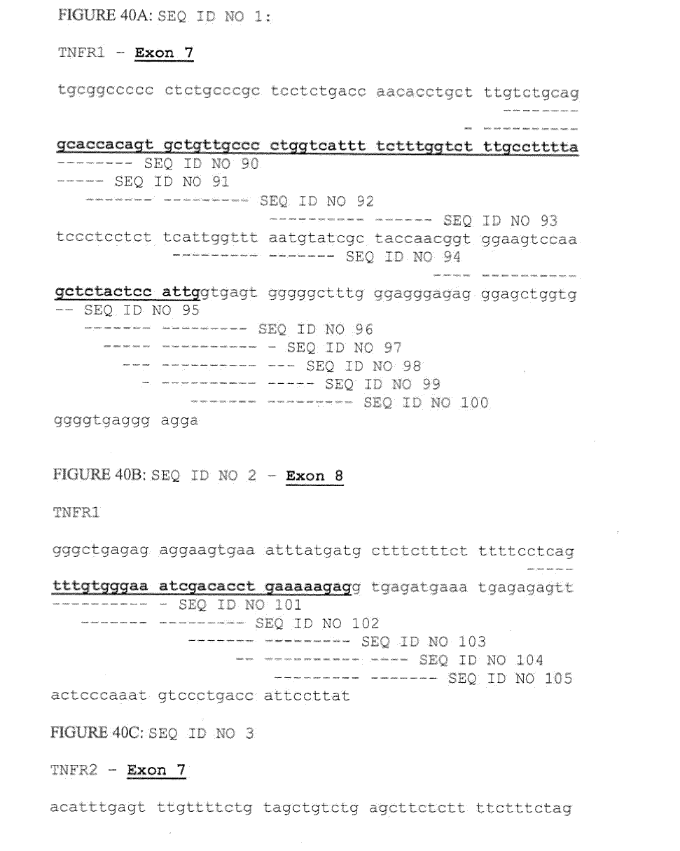

Human and mouse TNFR2 D7 with a His-tag (clones 1319-1 and 1144-4, respectively) were purified from the above media by affinity chromatography. HisPur™ cobalt spin columns (Pierce) were used to purify mouse and human TNFR2 Δ7 containing a His-tag from the above media. Approximately 32 ml, of media were applied to a 1 mL HisPur™ column equilibrated with 50 mM sodium phosphate, 300 mM sodium chloride, 10 mM imidazole buffer (pH 7.4) as recommended by the manufacturer. The column was then washed with two column volumes of the same buffer and protein was eluted with 1 mL of 50 mM sodium phosphate, 300 mM sodium chloride, 150 mM imidazole buffer (pH 7.4). Five mL of each eluate were analyzed by Western blot as described above (FIG. 39). TNFR2 Δ7 appears in the eluate and the multiple bands represent variably glycosylated forms of TNFR2 D7. As negative controls, the TNFR2 D7 proteins expressed from plasmids 1230-1 or 1145-1 which do not contain a His-tag where subjected to the above purification procedure. These proteins do not bind the affinity column and do not appear in the eluate (FIG. 39).

1. An oligomer of between 8 and 50 nucleobases in length, comprising of a contiguous nucleobase sequence which consists of between 8 and 50 nucleobases in length, wherein said contiguous nucleobase sequence is complementary to a corresponding region of contiguous nucleotides present in SEQ ID NO 1 or SEQ ID NO 2, SEQ ID NO 3, SEQ ID NO 4 and wherein said contiguous nucleobase sequence does not comprise 5 or more contiguous DNA (2′-deoxyribosnucleoside) monomer units.

2. The oligomer according to claim 1, wherein said oligomer is essentially incapable of recruiting RNAseH when formed in a duplex with a complex with a complementary mRNA molecule.

3. The oligomer according to claim 1 or 2, wherein the contiguous nucleobase sequence consists of a nucleobase sequence which is complementary to a corresponding region of SEQ ID NO 1 or SEQ ID NO 3.

4. The oligomer according to any one of claims 1-3, wherein said oligomer consists of said contiguous nucleobase sequence, and optionally 1, 2, or 3 nucleobases which may flank the contiguous nucleobase sequence at a position selected from: 5′ to the contiguous nucleobase sequence, 3′ to the contiguous nucleobase sequence, and both 5′ and 3′ to the contiguous nucleobase sequence.

5. The oligomer according to claim 4, wherein said 1, 2, or 3 nucleobases which flank the contiguous nucleobase sequence are nucleotides units, such as DNA or RNA units.

6. The oligomer according to any one of claims 1-5, wherein the linkage groups between the nucleobases of the contiguous nucleobase sequence are selected from the group consisting of phosphodiester, phosphorothioate and boranophosphate.

7. The oligomer according to any one of claims 1-6, wherein said contiguous nucleobase sequence comprises or consists of nucleotide analogues (X).

8. The oligomer according to claim 7, wherein the nucleotide analogues (X) are independently selected form the group consisting of: 2′-O-alkyl-RNA unit, 2′-OMe-RNA unit, 2′-MOE-RNA unit, 2′-amino-DNA unit, 2′-fluoro-DNA unit, LNA unit, PNA unit, HNA unit, INA unit.

9. The oligomer according to claim 7 or 8, wherein the contiguous nucleobase sequence comprises both nucleotide analogues (X) and nucleotides (x).

10. The oligomer according to any one of claims 7-9, wherein the contiguous nucleobase sequence comprises a subsequence comprising at least one nucleotide and at least one nucleotide analogue.

11. The oligomer according to claim 10, wherein the subsequence is selected from the group consisting of Xx, xX, Xxx, xXx, xxX, XXx, XxX, xXX, XXxX, XxXX, XxXX, xXXX, xxxX, xxXx, xXxx, Xxxx, XXXXx, XXXxX, XXxXX, XxXXX, xXXXX, xxxxX, xxxXx, xxXxx, xXxxx, Xxxxx, wherein said subsequence is optionally repeated.

12. The oligomer according to claim 11, wherein the repeated subsequence is repeated for the entire length of the contiguous nucleobase sequence, wherein, optionally the 5′ and/or 3′ repeat may be truncated.

13. The oligomer according to any one of claim 7-12 wherein the contiguous nucleobase sequence comprises said at least one LNA analogue unit and at least one further nucleotide analogue unit other than LNA.

14. The oligomer according to claim 13, wherein the contiguous nucleobase sequence consists of at least one sequence X¹X²X¹ or X²X¹X², wherein X¹ is LNA and X² is a nucleotide analogue other than LNA.

15. The oligomer according to claim 14, wherein the contiguous nucleobase sequence consists of alternative X¹ and X² units.

16. The oligomer according to any one of claims 7-15, wherein the nucleotide analogue units, are independently selected form the group consisting of: 2′-OMe-RNA units, 2′-fluoro-DNA units, and LNA units.

17. The oligomer according to claim 7-16, wherein the nucleotide analogue units (X) are LNA units.

18. The oligomer according to claim 7-17, wherein the LNA units are selected from the group consisting of oxy-LNA, amino-LNA, thio-LNA, and ena-LNA.

19. The oligomer according to claim 7-18, wherein the contiguous nucleobase sequence does not comprise a contiguous subsequence consisting of 5 or more contiguous nucleobases independently selected from DNA and alpha-L LNA units.

20. The oligomer according to claim 7-18, wherein the contiguous nucleobase sequence does not comprise a contiguous sub-sequence consisting of 5 or more contiguous nucleobases independently selected from DNA and alpha-L-oxy LNA units.

21. The oligomer according to any one of claims 7-20, wherein all the LNA units are in the beta-D configuration.

22. The oligomer according to any one of claims 1-21, wherein the length of the contiguous nucleobase sequence is between 8 and 16, such as 9, 10, 11, 12, 13, 14, 15 or 16 nucleobases, in length.

23. The oligomer according to any one of claims 1-22, wherein the length of the contiguous nucleobase sequence is between 8 and 15, such as 8, 9, 10, 11, 12, 13, 14, or 15 nucleobases, in length.

24. The oligomer according to any one of claims 1-23, wherein said contiguous nucleobase sequence is complementary to a corresponding region of contiguous nucleotides present in a sequence selected from the group consisting of: 51-164 of SEQ ID NO 1, 51-79 of SEQ ID NO 2, 51-127 of SEQ ID NO 3, and 51-85 of SEQ ID NO 4; or an equivalent position in SEQ ID NO 247-SEQ ID NO 250.

25. The oligomer according to any one of claims 1-23, wherein said contiguous nucleobase sequence is complementary to a corresponding region of contiguous nucleotides present in a sequence selected from the group consisting of: 1-50 of SEQ ID NO 1, 165-215 of SEQ ID NO 1, 1-50 of SEQ ID NO 2, 80-130 of SEQ ID NO 2, 1-50 of SEQ ID NO 3, 128-178 of SEQ ID NO 3, 1-50 of SEQ ID NO 4, and 86-136 of SEQ ID NO 4; or an equivalent position in SEQ ID NO 247-SEQ ID NO 250.

26. The oligomer according to any one of claims 1-23, wherein said contiguous nucleobase sequence comprises a nucleobase sequence which is complementary to an 5′ exon/intron 3′ or 3′ intron/exon 5′ border; or an equivalent position in SEQ ID NO 247-SEQ ID NO 250.

27. The oligomer according to claim 26, wherein said 5′ exon/intron 3′ or 3′ intron/exon 5′ border is selected from the group consisting of nucleobases 50-51 of SEQ ID NO 1, 164-165 of SEQ ID NO 1, 50-51 of SEQ ID NO 2, 79-80 of SEQ ID NO 2, 51-52 of SEQ ID NO 3, 129-139 of SEQ ID NO 3, 50-51 of SEQ ID NO 4, 81-82 of SEQ ID No 4; or an equivalent position in SEQ ID NO 247-SEQ ID NO 250.

28. The oligomer according to any one of claims 1-27, wherein said contiguous nucleobase sequence is identical to or is present in a nucleobase sequence present in a sequence selected from the group consisting of SEQ ID NO 74 to SEQ ID NO 105.

29. The oligomer according to claim 28, wherein said contiguous nucleobase sequence is identical to or is present in a nucleobase sequence selected from the group consisting of: SEQ ID NO 74, SEQ ID NO 75, SEQ ID NO 77, SEQ ID NO 78, SEQ ID NO 80, SEQ ID NO 82, and SEQ ID NO 84.

30. The oligomer according to claim 28, wherein said contiguous nucleobase sequence is identical to or is present in a nucleobase sequence selected from the group consisting of: SEQ ID NO 85, SEQ ID NO 86, SEQ ID NO 87, SEQ ID NO 88, and SEQ ID NO 89.

31. The oligomer according to any one of claims 1-30, wherein said contiguous nucleobase sequence comprises a nucleobase sequence which is complementary to a region of SEQ ID No 3 selected from nucleotides: 47-49, 54-56, and 122-124.

32. The oligomer according to any one of claims 1-31, wherein said contiguous nucleobase sequence is identical to or is present in a nucleobase sequence or a nucleobase motif sequence selected from the group consisting of: SEQ ID NO 130-SEQ ID No 145, SEQ ID NO 146-SEQ ID NO 161, and SEQ ID NO 162-177.

33. The oligomer according to claim 32, wherein the oligomer is selected from the group consisting of: SEQ ID NO 244-SEQ ID NO 246, SEQ ID NO 251-263, SEQ ID NO 264-SEQ ID NO 279, and SEQ ID NO 280-SEQ ID NO 295.

34. A conjugate comprising the oligomer according to any one of the claims 1-33 and at least one non-nucleotide moiety covalently attached to said oligomer.

35. A pharmaceutical composition comprising the oligomer according to any one of claims 1-33, or the conjugate according to claim 34, and a pharmaceutically acceptable carrier.

36. A method of altering the splicing of a TNFalpha receptor pre-mRNA mRNA, selected from TNFRSF1A or TNFRSF1A in a mammalian cell which expresses TNFRSF1A TNFalpha receptor or TNFRSF1B TNFalpha receptor, said method comprising administering oligomer according to any one of claims 1-33 or a conjugate according to claim 34, or the pharmaceutical composition according to claim 35 to the cell.

37. A method of preparing a soluble form of TNFRSF1A TNFalpha receptor or TNFRSF1B TNFalpha receptor in a mammalian cell which expresses said TNFalpha receptor, said method comprising administering the oligomer according to any one of claims 1-33 or a conjugate according to claim 34, or the pharmaceutical composition according to claim 35 to the cell.

38. The method according to claim 37, which further comprises the step of isolating or purifying the soluble form of the TNFalpha receptor TNFRSF1A or TNFRSF1B from said mammalian cell.

39. A method of increasing the expression of a soluble form of TNFRSF1A TNFalpha receptor or TNFRSF1B TNFalpha receptor in a mammalian cell which expresses said TNFalpha receptor, said method comprising administering the oligomer according to any one of claims 1-33 or a conjugate according to claim 34, or the pharmaceutical composition according to claim 35 to the cell.

40. The method according to any one of claims 36-39, wherein the method is performed in vitro or in vivo.

41. The use of an oligomer according to any one of claim 1-33, or conjugate according to claim 34 for the preparation of a medicament for the treatment of an inflammatory disease or condition.

42. An oligomer according to any one of claim 1-33 or a conjugate according to claim 34, for the treatment of an inflammatory disease or condition.

43. A method of treatment or prevention of an inflammatory disease or condition comprising the steps of administering the pharmaceutical composition according to claim 35 to a patient who is suffering from, or is likely to suffer from said inflammatory disease.

44. An isolated, or purified, soluble form of TNFalpha receptor comprises a deletion in the trans-membrane binding domain encoded by exon 7, wherein said TNFalpha receptor is selected from the TNFalpha receptor TNFRSF1A or TNFRSF1B.

45. The isolated, or purified, soluble form of TNFalpha receptor according to claim 44, wherein said TNFalpha receptor lacks the trans-membrane binding domain encoded by exon 7.

46. The isolated, or purified, soluble form of TNFalpha receptor according to claim 44 or 45, wherein the TNFalpha receptor is the human TNFR1 TNFalpha receptor (residues 1-455, or residues 30-455 of SEQ ID NO 123, or a variant, fragment or homologue thereof), wherein said deletion is between residues 209 and 246.

47. The isolated, or purified, soluble form of TNFalpha receptor according to claim 46 which has a sequence consisting of residues 1-208 or residues 30-208 of SEQ ID NO 119, or is a variant, fragment or homologue thereof.

48. The isolated, or purified, soluble form of TNFalpha receptor according to claim 44 or 45, wherein the TNFalpha receptor is the human TNFR2 TNFalpha receptor (residues 1-435, or residues 23-435 of SEQ ID NO 127, or a variant, fragment or homologue thereof, wherein said deletion is between residues 263 and 289.

49. The isolated, or purified, soluble form of TNFalpha receptor according to claim 48 which has a sequence consisting of residues 1-262 or 23-262 of SEQ ID NO 127, or is a variant, fragment or homologue thereof.

50. A nucleic acid encoding the soluble form of TNFalpha receptor according to any one of claims 44-49.

51. The nucleic acid according to claim 50, wherein nucleic acid is selected from the group consisting of: nucleotides 1-1251 of SEQ ID NO 121, 88-1251 of SEQ ID NO 121, 1-1305 of SEQ ID NO 125 and 67-1305 of SEQ ID NO 125.

52. A vector comprising the nucleic acid according to claims 50 or 51.

53. The vector according to claim 52, wherein said vector comprises an expression cassette capable of driving the expression of said nucleic acid in a host cell.

54. A host cells comprising the nucleic acid according to claim 50 or 51, or the vector according to claim 52 or 53.

55. A method for the preparation of a soluble form of TNFalpha receptor, said method comprising the step of culturing the host cell according to claim 54 under conditions which allow the expression of said nucleic acid, and subsequently isolating said soluble form of TNFalpha receptor from said host cells.

56. A pharmaceutical composition comprising the isolated or purified soluble form of TNFalpha receptor according to any one of claims 44-49, or as prepared according to claim 55, and a pharmaceutically acceptable carrier.

57. The use of the isolated or purified soluble form of TNFalpha receptor according to any one of claim 44-49, or as prepared according to claim 56, for the preparation of a medicament for the treatment of an inflammatory disease or condition.

58. The isolated or purified soluble form of TNFalpha receptor according to any one of claim 44-49, or as prepared according to claim 57, for the treatment of an inflammatory disease or condition.

59. A method of treatment or prevention of an inflammatory disease or condition comprising the steps of administering the pharmaceutical composition according to claim 56 to a patient who is suffering from, or is likely to suffer from said inflammatory disease.

60. The oligomer of any of claims 1-23 wherein the oligomer consists of 8-50 nucleobases.

61. The oligomer of any of claims 1-23 wherein the nucleobase sequence has the same base sequence as any 8 to 50 contiguous nucleotides of any of SEQ ID NO:247-250.

62. The oligomer of any of claims 1-23 consisting of 9, 10, 11, 12, 13, 14 or 15 nucleobases.

63. The oligomer of any of claims 1-23 wherein the nucleobase sequence consists of 9, 10, 11, 12, 13, 14 or 15 nucleobases.

64. The oligomer of any of claims 1-23 wherein the nucleobase sequence consists of 8 to 50 contiguous nucleobases.
